(19) United States
(12) Patent Application Publication
(10) Pub. No.: US 2021/0233611 A1
(43) Pub. Date: Jul. 29, 2021

(54) CLASSIFICATION AND PROGNOSIS OF PROSTATE CANCER

(71) Applicant: UEA ENTERPRISES LIMITED, Norwich (GB)

(72) Inventors: Daniel Simon Brewer, Norwich Norfolk (GB); Bogdan-Alexandru Luca, Norwich Norfolk (GB); Vincent Moulton, Norwich Norfolk (GB); Colin Cooper, Norwich Norfolk (GB)

(21) Appl. No.: 17/046,829

(22) PCT Filed: Apr. 12, 2019
(86) PCT No.: PCT/EP2019/059451

§ 371 Date: Oct. 12, 2020

(30) Foreign Application Priority Data
Apr. 12, 2018 (GB) 1806064.0

(51) Int. Cl. G16B 25/10; G16B 40/30

(52) U.S. Cl. CPC G16B 25/10; G16B 40/30

(57) ABSTRACT

The present invention relates to the classification of prostate cancers using samples from patients. Classification is achieved using a novel analysis method that uses less computing power than methods of the prior art. In particular, the invention provides new methods for classifying cancers to make a determination of risk of cancer progression (for example in early cancer), to identify patient populations that may be susceptible to particular treatments and to present opportunities (for example to provide tailored treatment regimens), or to identify patient populations that do not require treatment. The methods of the invention may include identifying potentially aggressive cancers to determine which cancers are or will become aggressive (and hence require treatment) and which will remain indolent (and will therefore not require treatment). The present invention is therefore useful to identify a patient's prognosis and identify those with good or poor prognoses. The present method also allows the identification of patient populations that may be susceptible to treatment with particular drug treatments.

The present invention relates to the classification of prostate cancers using samples from patients. Classification is achieved using a novel analysis method that uses less computing power than methods of the prior art. In particular, the invention provides new methods for classifying cancers to make a determination of risk of cancer progression (for example in early cancer), to identify patient populations that may be susceptible to particular treatments and to present opportunities (for example to provide tailored treatment regimens), or to identify patient populations that do not require treatment. The methods of the invention may include identifying potentially aggressive cancers to determine which cancers are or will become aggressive (and hence require treatment) and which will remain indolent (and will therefore not require treatment). The present invention is therefore useful to identify a patient's prognosis and identify those with good or poor prognoses. The present method also allows the identification of patient populations that may be susceptible to treatment with particular drug treatments.

### CROSS-REFERENCE TO RELATED APPLICATIONS

This application is the U.S. national phase application filed under 35 U.S.C. § 371 claiming benefit to International Patent Application No. PCT/EP2019/059451, filed on Apr. 12, 2019, which claims priority to GB Patent Application No. 1806064.0, filed Apr. 12, 2018, the disclosures of which are incorporated herein by reference in their entirety.

### BACKGROUND

A common method for the diagnosis of prostate cancer is the measure of prostate specific antigen (PSA) in blood. However, as many as 50-80% of PSA-detected prostate cancers are biologically irrelevant, that is, even without treatment, they would never have caused any symptoms. Radical treatment of early prostate cancer, with surgery or radiotherapy, should ideally be targeted to men with significant cancers, so that the remainder, with biologically ‘irrelevant’ disease, are spared the side-effects of treatment. Accurate prediction of individual prostate cancer behaviour at the time of diagnosis is not currently possible, and immediate radical treatment for most cases has been a common approach. Put bluntly, many men are left impotent or incontinent as a result of treatment for a ‘disease’ that would not have troubled them. A large number of prognostic biomarkers have been proposed for prostate cancer. A key question is whether these biomarkers can be applied to PSA-detected, early prostate cancer to distinguish the clinically significant cases from those with biologically irrelevant disease. Validated methods for detecting aggressive cancer early could lead to a paradigm-shift in the management of early prostate cancer. For patients with early and more advanced disease there is also a need to identify patients who may be sensitive to particular drug treatments.

A critical problem in the clinical management of prostate cancer is that it is highly heterogeneous. Accurate prediction of individual cancer behaviour is therefore not achievable at the time of diagnosis leading to substantial overtreatment. It remains an enigma that, in contrast to many other cancer types, stratification of prostate cancer based on unsupervised analysis of global expression patterns has not been possible: for breast cancer, for example, ERBB2 overexpressing, basal and luminal subgroups can be identified.

Driven by technological advances and decreased costs, a plethora of genomic datasets now exist. This is illustrated by the availability of expression data from over 1.3 million samples from the Gene Expression Omnibus¹ and DNA sequence data on 25,000 cases from the International Cancer Genome Consortium². Such datasets have been used as the raw material for the discovery of disease sub-classes using a variety of mathematical approaches. Hierarchical clustering³, k-means clustering⁴, and self-organising maps⁵ have been applied to expression datasets leading, for example, to the discovery of five molecular breast cancer types (Basal, Luminal A, Luminal B, ERBB2-overexpressing, and Normal-like)⁶. The inherent shortcoming of the approaches mentioned above is the implicit assumption of sample assignment to a particular cluster or group. Such analyses are in complete contrast to the well documented heterogeneous composition of most individual cancer samples.

There remains in the art a need for a more reliable diagnostic test for prostate cancer and to better assist in distinguishing between aggressive cancer, which may require treatment, and non-aggressive cancer, which perhaps can be left untreated and spare the patient any side effects from unnecessary interventions. There also remains a need in the art to provide methods of prostate cancer classification to identify patient populations that have different treatment sensitives to tailor treatment regimens to patients that will be susceptible to treatment.

### SUMMARY OF THE INVENTION

The present invention provides algorithm-based molecular diagnostic assays for classifying prostate cancer and thereby providing a cancer prognosis. In some embodiments, the expression statuses of certain genes may be used alone or in combination to classify the cancer. The algorithm-based assays and associated information provided by the practice of the methods of the present invention facilitate optimal treatment decision making in prostate cancer. For example, such a clinical tool would enable physicians to identify patients who have a high risk of having aggressive disease and who therefore need radical and/or aggressive treatment. It would also enable physicians to identify patients that do not require treatment, or require treatment with a particular drug according to the drug sensitivity of the classification of cancer assigned to that patient.

The present invention improves on previous attempts to classify in particular prostate cancers by the identification, for the first time, of up to 8 different prostate cancer classifications (also referred to herein as cancer expression signatures), including at least three new clinically and/or genetically distinct subtypes of prostate cancer. Each classification of cancer provides a different insight into the expected progression (or not, as the case may be) of a patient's cancer, as determined using a patient sample. The present invention shows 8 different cancer populations, referred to S1 to S8, including a poor clinical outcome in prostate cancer that is dependent on the proportion of cancer containing a cancer expression signature that is associated with a poor prognosis, for example the cancer classification referred to herein as S7 or DESNT.

The present invention also improves on previous attempts to classify prostate cancer by providing a novel analysis method for detecting 8 cancer groups whilst reducing the computing power required to conduct the classification to enable a faster and easier classification of a patient's cancer sample.

Unsupervised analysis of prostate cancer transcriptome profiles using the above approaches failed to identify robust disease categories that have distinct clinical outcomes^{7,8}. Noting that prostate cancer samples derived from genome wide studies frequently harbour multiple cancer lineages, and often have heterogeneous compositions⁹⁻¹², the inventors applied an unsupervised learning method called Latent Process Decomposition (LPD)¹³. LPD (closely related to Latent Dirichlet Allocation¹⁶) is a mixed membership model in which the expression profile for a cancer is represented as a combination of underlying latent processes. Each latent process (equivalent to a cancer expression signature, cancer group, cancer classification or cancer population as used herein) is considered as an underlying functional state or the expression profile of a particular component of the cancer. A given sample can be represented over a number of these underlying functional states, or just one such state. The appropriate number of processes to use (the model complexity) is determined using the LPD algorithm by maximising the probability of the model given the data.

The present inventors have applied a Bayesian clustering procedure called Latent Process Decomposition (LPD, Simon Rogers, Mark Girolami, Colin Campbell, Rainer Breitling, “The Latent Process Decomposition of cDNA Microarray Data Sets”, IEEE/ACM Transactions on Computational Biology and Bioinformatics, vol. 2, no. 2, pp. 143-156, April-June 2005, doi:10.1109/TCBB.2005.29) to classify cancer samples, specifically prostate cancer samples, and have identified 8 different cancer classifications. The results demonstrate the existence of novel categories of human prostate cancer, and assists in the targeting of therapy, helping avoid treatment-associated morbidity in men with indolent disease. Unlike in Rogers et al., the present inventors identify 8 different consistent cancer classifications and performed an analysis to determine the correlation of the groups with survival and to provide a definition of signature genes for each signature. The inventors surprisingly identified that two different prostate cancer datasets both could be decomposed using an LPD analysis into 8 different cancer classifications (also referred to herein as processes, groups or signatures), and that the 8 different cancer classifications were substantially identical between the two datasets, despite the different input data from the two different datasets. In doing so, the present inventors identified 8 cancer classifications that can be applied globally to all prostate cancer samples and used to classify any patient sample. Since some of the prostate cancer classifications are associated with different cancer prognoses, the classification of a patient sample is informative regarding the treatment steps that should be taken (if any). The present inventors also discovered that the contribution of the different groups to a given expression profile can be used to determine the prognosis of the cancer, optionally in combination with other markers for prostate cancer such as tumour stage, Gleason score and PSA. The contribution of each group (i.e. cancer classification) to a patient's overall cancer is a continuous variable, and the level of contribution of a given group to a patient expression profile is informative about the cancer's need for and sensitivity to certain treatments. Notably, the methods of the present invention are not simple hierarchical clustering methods and allow a much more detailed and accurate analysis of patient samples that such prior art methods.

For the first time, the present inventors have provided a method that allows a reliable classification of cancer and prediction of cancer progression, whereas methods of the prior art could not be used to detect cancer progression, since there was nothing to indicate such a correlation could be made. The present inventors also provide, for the first time, a method of analysis of patient samples that is quick and easy to execute without requiring the entire LPD method (which requires significant computing power) to be conducted each time.

The present inventors have also used additional mathematical techniques to provide further methods of prognosis and diagnosis, and also provide biomarkers and biomarker panels useful in classifying patient cancer samples, including identifying patients with a poor prognosis or indeed with a good prognosis.

In a first aspect of the invention, there is provided a method of classifying prostate cancer or predicting prostate cancer progression in a patient, comprising:
a) providing a set of reference parameters, wherein the reference parameters are obtained from a Latent Process Decomposition (LPD) analysis performed on a reference dataset, the reference dataset comprising A expression profiles, each expression profile comprising the expression status of G genes, wherein the reference dataset is decomposed using the LPD analysis into K different cancer expression signatures;
b) obtaining or providing the expression status of G genes in a sample obtained from the patient to provide a patient expression profile, wherein the G genes in the patient expression profile are the same genes of the reference dataset used to provide the set of reference parameters; and
c) classifying the cancer or predicting cancer progression by determining the contribution of each different cancer classification to the patient expression profile using the set of reference parameters provided in step (a).

In a second aspect of the invention, there is provided a method of classifying prostate cancer or predicting prostate cancer progression, comprising:
a) providing one or more reference datasets where the cancer classification of each patient sample in the datasets is known (for example as determined by LPD analysis);
b) selecting from this dataset a plurality of genes;
c) applying a LASSO logistic regression model analysis on the selected genes to identify a subset of the selected genes that are predictive of each cancer classification;
d) using the expression status of this subset of selected genes to apply a supervised machine learning algorithm on the dataset to obtain a predictor for each cancer classification;
e) providing or determining the expression status of the subset of selected genes in a sample obtained from the patient to provide a patient expression profile;
f) optionally normalising the patient expression profile to the reference dataset(s); and
g) applying the predictor to the patient expression profile to classify the cancer or predict cancer progression.

In some embodiments of the invention, the cancer classifications of part (a) are the 8 prostate cancer classifications identified for the first time in the present invention.

In a third aspect of the invention, there is provided a method of classifying prostate cancer or predicting prostate cancer progression, comprising:
a) providing one or more reference datasets where the cancer classification of each patient sample in the datasets is known (for example as determined by LPD analysis);
b) selecting from this dataset a plurality of genes, wherein the plurality of genes comprises at least 5, at least 10, at least 20, at least 30, at least 40, at least 50, at least 100, or at least 150 genes selected from the group listed in Table 2
c) optionally:
   i. determining the expression status of at least 1 further, different, gene in the patient sample as a control, wherein the control gene is not a gene listed in Table 2 and
   ii. determining the relative levels of expression of the plurality of genes and of the control gene(s);
d) using the expression status of those selected genes to apply a supervised machine learning algorithm on the dataset to obtain a predictor for cancer classification;
e) providing or determining the expression status of the same plurality of genes in a sample obtained from the patient to provide a patient expression profile;
f) optionally normalising the patient expression profile to the reference dataset; and
g) applying the predictor to the patient expression profile to classify the cancer, or to predict cancer progression.

In a fourth aspect of the invention, there is provided a method of classifying prostate cancer or predicting prostate cancer progression, comprising:
a) providing a reference dataset wherein the cancer classification of each patient sample in the dataset is known (for example as determined by LPD analysis);
b) selecting from this dataset of a plurality of genes;
c) using the expression status of those selected genes to apply a supervised machine learning algorithm on the dataset to obtain a predictor for cancer classification;
d) providing or determining the expression status of the same plurality of genes in a sample obtained from the patient to provide a patient expression profile;
e) optionally normalising the patient expression profile to the reference dataset; and
f) applying the predictor to the patient expression profile to classify the cancer, or to predict cancer progression.

In a fifth aspect of the invention, there are provided a series of biomarker panels that are useful in the classification of prostate cancer, or a predictor for the progression of cancer.

In a further aspect of the invention there is provided a method of diagnosing, screening or testing for prostate cancer, or for providing a prognosis for prostate cancer, comprising detecting, in a sample, the level of expression of all or a selection of the genes from the biomarker panels. In some embodiments, the biological sample is a prostate tissue biopsy (such as a suspected tumour sample), saliva, a blood sample, or a urine sample. Preferably the sample is a tissue sample from a prostate biopsy, a prostatectomy specimen (removed prostate) or a TURP (transurethral resection of the prostate) specimen.

There is also provided one or more genes in the biomarker panels for use in detecting or diagnosing prostate cancer, or for providing a prognosis for prostate cancer. There is also provided the use of one or more genes in the biomarker panels in methods of detecting or diagnosing prostate cancer, or for providing a prognosis for prostate cancer, as well as methods of detecting, diagnosing or providing a prognosis for such cancers using one or more genes in the biomarker panels.

There is also provided one or more genes in the biomarker panels for use in predicting progression of prostate cancer. There is also provided the use of one or more genes in the biomarker panel in methods of predicting progression of prostate cancer, as well as methods of predicting prostate cancer progression using one or more genes in the biomarker panels.

There is also provided one or more genes in the biomarker panels for use in classifying cancer (such as prostate cancer). There is also provided the use of one or more genes in the biomarker panel in classifying prostate cancer, as well as methods of classifying prostate cancer using one or more genes in the biomarker panels.

There is also provided one or more genes in the biomarker panels for use in determining or predicting a patient's response to a therapy, such as a prostate cancer drug therapy. There is also provided the use of one or more genes in the biomarker panel in determining or predicting a patient's response to a therapy, such as a prostate cancer drug therapy, as well as methods of determining or predicting a patient's response to a therapy, such as a prostate cancer drug therapy, using one or more genes in the biomarker panels.

There is further provided a kit of parts for testing for, classifying or prognosing prostate cancer comprising a means for detecting the expression status of one or more genes in the biomarker panels in a biological sample. The kit may also comprise means for detecting the expression status of one or more control genes not present in the biomarker panels.

There is still further provided methods of diagnosing aggressive cancer, methods of classifying cancer, methods of prognosing cancer, and methods of predicting cancer progression comprising detecting the level of expression of one or more genes in the biomarker panels in a biological sample. Optionally the method further comprises comparing the expression levels of each of the quantified genes with a reference.

In a still further aspect of the invention there is provided a method of treating prostate cancer in a patient, comprising proceeding with treatment for prostate cancer if aggressive prostate cancer or cancer with a poor prognosis is diagnosed or suspected. In the invention, the patient has been diagnosed as having aggressive prostate cancer or as having a poor prognosis using one of the methods of the invention. In some embodiments, the method of treatment may be preceded by a method of the invention for diagnosing, classifying, prognosing or predicting progression of cancer (such as prostate cancer) in a patient, or a method of identifying a patient with a poor prognosis for prostate cancer, (i.e. identifying a patient with DESNT prostate cancer). Also provided are methods of treating prostate cancer in a patient, comprising administering a treatment to a patient that has been identified using a classification method described herein as being sensitive to or suitable for the particular therapy.

### BRIEF DESCRIPTION OF THE FIGURES

FIG. 1. LPD decomposition of the MSKCC dataset. (a) Samples are represented in all eight processes and height of each bar corresponds to the proportion (Gamma, vertical axis) of the signature that can be assigned to each LPD process. The seventh row illustrates the percentage of the DESNT expression signature identified in each sample. (b) Bar chart showing the proportion of DESNT cancer present in each sample. (c,d) Pie charts showing the composition of individual cancers. DESNT is in red. Other LPD groups are represented by different colours as indicated in the key. The number next the pie chart indicates which cancer it represents from the bar chart above. Individual cancers were assigned as a “DESNT cancer” when the DESNT signature was the most abundant; examples are shown in the right hand box (d, DESNT). Many other cancers contain a smaller proportion of DESNT cancer and are predicted also to have a poor outcome: examples shown in larger box (c, SOME DESNT).

FIG. 2. Stratification of prostate cancer based on the percentage of DESNT cancer present. For these analyses the data from the MSKCC, CancerMap, CamCap and Stephenson datasets were combined (n=503). (a) Plot showing the contribution of DESNT signature to each cancer and the division into 4 groups. Group 1 samples have less than 0.1% of the DESNT signature. (b) Kaplan-Meier plot showing the Biochemical Recurrence (BCR) free survival based on proportion of DESNT cancer present as determined by LPD. Number of cancers in each Group are indicated (bottom right) and the number of PCR failures in each group are show in parentheses. The definition of Groups 1-4 is shown in FIG. 2a. Cancers with Gamma values up to 25% DESNT (Group 2) exhibited poorer clinical outcome (X²-test, P=0.011) compared to cancers lacking DESNT (<0.1%). Cancers with the intermediate (0.25 to 0.45) and high (>0.45) values of Gamma also exhibited significantly worse outcome (respectively P=2.63×10⁻⁵ and P=8.26×10⁻⁹ compare to cancers lacking DESNT. The combined log-rank P=1.28×10⁻⁸.

FIG. 3. Nomogram model developed to predict PSA free survival at 1, 3, 5 and 7 years using DESNT Gamma. Assessing a single patient each clinical variable has a corresponding point score (top scales). The point scores for each variable are added to produce a total points score for each patient. The predicted probability of PSA free survival at 1, 3, 5 and 7 years can be determined by drawing a vertical line from the total points score to the probability scales below.

FIG. 4. Correlation in expression profiles between MSKCC and CancerMap LPD groups. Correlations of the average levels of gene expression for cancers assigned to each LPD group are presented. The expression levels of each gene have been normalised across all samples to mean 0 and standard deviation 1. Even for the lower Pearson Coefficients the correlation is highly statistically significant (Pearson's product-moment correlation test).

FIG. 5. Prediction of clinical outcome according to OAS-LPD group. (a-c) Kaplan-Meier plots showing PSA free survival outcomes for the cancers assigned to LPD groups in analyses of the combine MSKCC, CancerMap, CamCap and Stephenson datasets: (a) comparison of all LPD groups; (b) cancers assign to LPD4 compared to cancers assigned to all other LPD groups; (c) cancers assign to DESNT compared to cancers assigned to all other LPD groups. (d-f) Kaplan-Meier plots showing PSA free survival outcomes for ERG-rearrangement positive cancers in LPD3 compared to all other cancers for the CancerMap, CamCap and TCGA datasets.

FIG. 6. OAS-LPD sub-groups in The Cancer Genome Atlas Dataset. Cancers were assigned to subgroups based on the most prominent signature as detected by OAS-LPD. The types of genetic alteration are shown for each gene (mutations, fusions, deletions, and over-expression). Clinical parameters including biochemical recurrence (BCR) are represented at the bottom together with groups for iCluster, methylation, somatic copy number alteration (SVNA), and messenger RNA (mRNA)²⁰. Comparison of the frequency of genetic alterations present in each subgroup are shown in Table 7.

FIG. 7. A classification framework for human prostate cancer. Based on the analyses of genetic and clinical correlations we consider that there is good evidence for the existence of S3, S4 and S5 as separate cancer categories, moderate evidence of the existence of S6 and S8 (based on alteration of expression only) and weak evidence for 51.

FIG. 8. Correlation of metastatic cancer with OAS-LPD category. (a) OAS-LPD assignments were determined based on analysis of expression profiles of primary cancers as shown in FIG. 11. The frequency of cancers associated with developing metastases in each LPD category is shown for the Erho et al³⁹ (upper panel) and MSKCC⁸ (lower panel) datasets. (b) Expression profiles for the 19 metastases reported as part of the MSKCC dataset were subject to OAS-LPD. In all cases LPD7(DESNT) was the dominant expression signature detected.

FIG. 9. Example computer apparatus.

FIG. 10. Cox Model for DESNT cancers assessed by LPD. (a) graphical representation of HR for each covariate and 95% confidence interavals of HR. (b) HR, 95% CI and Wald test statistics of the Cox model. (c) Calibration plots for the internal validation of the nomogram, using 1000 bootstrap resamples. Solid black line represents the apparent performance of the nomogram, blue line the bias-corrected performance and dotted line the ideal performance. (d) Calibration plots for the external validation of the nomogram using the CamCap dataset. Solid line corresponds to the observed performance and dotted line to the ideal performance.

FIG. 11. Add One Sample Latent Process Decomposition (OAS-LPD) for eight prostate cancer transcriptome datasets. See FIG. 1 for a description of the plots with the exception that in this Figure the different colours denote different Gleason Sums. Vertical axis is the fraction of the sample (Gamma).

FIG. 12. Cox Model for DESNT cancers assessed by OAS-LPD. (a) graphical representation of HR for each covariate and 95% confidence intervals of HR. (b) HR, 95% CI and Wald test statistics of the Cox model. (c) Calibration plots for the internal validation of the nomogram, using 1000 bootstrap resamples. Solid black line represents the apparent performance of the nomogram, blue line the bias-corrected performance and dotted line the ideal performance. (d) Calibration plots for the external validation of the nomogram using the CamCap dataset. Solid line corresponds to the observed performace and dotted line to the ideal performance.

FIG. 13. Nomogram model developed to predict PSA free survival at 1, 3, 5 and 7 years for DESNT cancer assessed by OAS-LPD. Assessing a single patient each clinical variable has a corresponding point score (top scales). The point scores for each variable are added to produce a total points score for each patient. The predicted probability of PSA free survival at 1, 3, 5 and 7 years can be determined by drawing a vertical line from the total points score to the probability scales below.

FIG. 14. GO pathway over-representation analysis for the lists of differentially expressed genes in each process. For each gene set, up to 5 pathways with the lowest p-values are represented. Blue nodes correspond to pathways, red nodes to genes, and the vertices indicate the involvement of the gene in the pathway. The size of blue nodes is inversely proportional to the over-representation p-value.

### DETAILED DESCRIPTION OF THE INVENTION

The present invention provides methods, biomarker panels and kits useful in predicting cancer progression.

LPD-Derived Methods

In one embodiment of the invention, there is provided a method of classifying prostate cancer or predicting prostate cancer progression in a patient, comprising:
a) providing a set of reference parameters, wherein the reference parameters are obtained from a Latent Process Decomposition (LPD) analysis performed on a reference dataset, the reference dataset comprising A expression profiles, each expression profile comprising the expression status of G genes, wherein the reference dataset is decomposed using the LPD analysis into K different cancer expression signatures;
b) obtaining or providing the expression status of G genes in a sample obtained from the patient to provide a patient expression profile, wherein the G genes in the patient expression profile are the same genes of the reference dataset used to provide the set of reference parameters; and
c) classifying the prostate cancer or predicting prostate cancer progression by determining the contribution of each different cancer expression signature to the patient expression profile using the set of reference parameters provided in step (a).

This method is of particular relevance to prostate cancer, but it can be applied to other cancers. Such a method may be referred to herein as Method 1.

Each cancer expression signature correlates to a cancer classification, that may be distinguishable from other cancer classifications according to, for example, the clinical outcome and/or the gene expression (and optionally mutation) profile of the cancer.

The step of classifying the cancer may comprise determining the cancer expression signature that contributes the most to the patient expression profile and assigning the patient cancer to that cancer classification. In such a situation, the cancer classification corresponding to the most dominant cancer expression signature is assigned to the patient sample and appropriate treatment actions can take place accordingly.

In some embodiments, the step of classifying the cancer or predicting cancer progression comprises splitting the patient expression profile between the gene expression profiles for each cancer expression signature. Therefore, the method provides information regarding the contribution of each cancer expression signature to the patient expression profile(s) being classified.

In one embodiment of the invention, providing a set of reference parameters may comprise providing the reference dataset comprising A expression profiles and G genes for each expression profile; and performing LPD analysis on the reference dataset to classify each expression profiles into K cancer classifications. In other words, in some embodiments of the invention, the step of conducting LPD analysis on a reference dataset to provide the reference variables is part of the method. However, in preferred embodiments, the LPD has already been conducted on a reference dataset, and hence the computing power required for an LPD analysis is not needed to conduct the invention. Accordingly, in preferred embodiments, the method does not comprise a step of conducting LPD analysis on the reference dataset.

The reference parameters may be derived from a representative (e.g. average) LPD analysis. For example, the representative LPD analysis may be the LPD run with the survival log-rank p-value closest to the modal value. The reference parameters may therefore represent the representative or average values from a plurality of LPD runs.

The parameter K represents the number of cancer expression signatures (also referred to herein as cancer classifications, processes or states), and this may be different for the different types of cancer being analysed. In one embodiment, in particular embodiments relating to prostate cancer, K may be 7, 8 or 9. In a preferred embodiment, K is 8. Indeed, the present inventors have surprisingly identified, for the first time, 8 different cancer expression signatures that can be used to define prostate cancer in humans. Each of the 8 different cancer expression signatures correlates with a different cancer classification. In the context of LPD, K may be preferred to as a “process”.

The methods of the invention rely on a Bayesian clustering analysis referred to in the art as a latent process decomposition (LPD) analysis. Such mathematical models are known to a person of skill in the art and are described in, for example, Simon Rogers, Mark Girolami, Colin Campbell, Rainer Breitling, “The Latent Process Decomposition of cDNA Microarray Data Sets”, IEEE/ACM Transactions on Computational Biology and Bioinformatics, vol. 2, no. 2, pp. 143-156, April-June 2005, doi:10.1109/TCBB.2005.29. The LPD analysis groups the patients into “processes”. The present inventors have surprisingly discovered that when the LPD analysis is carried out using genes whose expression levels are known to vary across prostate cancers, 8 different cancer classifications are identified, at least 3 of these being associated with particular clinical outcomes.

When an LPD analysis is carried out on the reference dataset or reference datasets, which includes, for a plurality of patients, information on the expression levels for a number of genes whose expression levels vary significantly across prostate cancers, it determines the contribution of each underlying cancer expression signature or “process” (correlating to different cancer classifications) to each expression profile in the dataset. The inventors have surprisingly found that for prostate cancer, expression profiles can reliably be decomposed into 8 different cancer expression signatures or processes. An assessment can then be made about which processes a given expression profile should be assigned to. For example, cancers may be assigned to individual processes based on their highest pᵢ value, wherein pᵢ is the contribution of each process i to the expression profile of an individual cancer. The sum of pᵢ over all processes=1. However, the highest pᵢ value does not always need to be used and pᵢ can be defined differently, and skilled person would be aware of possible variations. For example, pᵢ can be at least 0.1, at least 0.2, at least 0.3, at least 0.4 or preferably at least 0.5. However, preferably, a cancer will be assigned to a process according to the process having the highest contribution to the overall expression profile.

Furthermore, for the first time the present inventors have developed a method that uses a framework provided for by the LPD analysis of a reference dataset to apply a simplified algorithm to a patient expression profile requiring a diagnosis or prognosis.

Choice and Number of Genes

The number of expression profiles in the reference dataset and the number of genes in each expression profile is not fixed. However, the larger the reference dataset and the higher the number of genes in each expression profile in the reference dataset, the more informative and accurate the method will be. In some embodiments, A is at least 100 (i.e. there are at least 100 expression profiles in the reference dataset) and G is at least 50 (i.e. there are at least 50 genes in each expression profile). Preferably, G is at least 500.

Of course, each expression profile in a given dataset does not have to include exactly all the same genes as all the other expression profiles in the dataset. Rather, there simply needs to be an overlapping set of genes across the expression profiles in the dataset. Therefore, the G genes are common to all A expression profiles in the reference dataset (allowing a comparison between the different expression profiles to be made and an informative analysis to be undertaken). The methods may also use a combination of reference datasets. In such situations, G may represent the genes that are common across all of the expression profiles in all of the datasets.

The choice of which genes to include in the analysis can vary. Preferably, the genes are genes whose expression levels are known to vary across cancers. For example, the level of expression may be determined for at least 50, at least 100, at least 200 or most preferably at least 500 genes that are known to vary across cancers. The skilled person can determine which genes should be measured, for example using previously published dataset(s) for patients with cancer and choosing a group of genes whose expression levels vary across different cancer samples. In particular, the choice of genes is determined based on the amount by which their expression levels are known to vary across difference cancers.

Variation across cancers refers to variations in expression seen for cancers having the same tissue origin (e.g. prostate, breast, lung etc). For example, the variation in expression is a difference in expression that can be measured between samples taken from different patients having cancer of the same tissue origin. When looking at a selection of genes, some will have the same or similar expression across all samples. These are said to have little or low variance. Others have high levels of variation (high expression in some samples, low in others).

A measurement of how much the expression levels vary across prostate cancers can be determined in a number of ways known to the skilled person, in particular statistical analyses. For example, the skilled person may consider a plurality of genes in each of a plurality of cancer samples and select those genes for which the standard deviation or inter-quartile range of the expression levels across the plurality of samples exceeds a predetermined threshold. The genes can be ordered according to their variance across samples or patients, and a selection of genes that vary can be made. For example, the genes that vary the most can be used, such as the 500 genes showing the most variation. Of course, it is not vital that the genes that vary the most are always used. For example, the top 500 to 1000 genes could be used. Generally, the genes chosen will all be in the top 50% of genes when they are according to variance. What is important is the expression levels vary across the reference dataset. The selection of genes is without reference to clinical aggression. This is known as unsupervised analysis. The skilled person is aware how to select genes for this purpose. In some embodiments, the method comprises an unsupervised analysis. In some embodiments, the genes selected for the analysis in the methods of the invention are selected without reference to any correlation between those genes and clinical aggression of the cancer (such as prostate cancer).

The methods of the invention may be conducted on a single expression profile from a single patient. Alternatively, two or more expression profiles from different patients undergoing diagnosis could be used. Such an approach is useful when diagnosing a number of patients simultaneously. The method may include a step of assigning a unique label to each of the patient expression profiles to allow those expression profiles to be more easily identified in the analysis step.

In some embodiments, in particular those relating to prostate cancer, the level of expression is determined for a plurality of genes selected from the list in Table 1.

In some embodiments, the method may involve providing or determining the level of expression at least 20, at least 50, at least 100, at least 200 or at least different 500 genes from the patient expression profile, wherein the genes are selected from the list in Table 1. As the number of genes increases, the accuracy of the test may also increase, although 500 genes should be more than enough to conduct the analysis. In a preferred embodiment, at least all 500 genes are selected from the list in Table 1. However, the method does not need to be restricted to the genes of Table 1.

In some cases, information on the level of expression of many more genes in the patent sample may be obtained, such as by using a microarray that determines the level of expression of a much larger number of genes. It is even possible to obtain the entire transcriptome. However, it is only necessary to carry out the subsequent analysis steps on a subset of genes whose expression levels are known to vary across prostate cancers. Preferably, the genes used will be those whose expression levels vary most across prostate cancers (i.e. expression varies according to cancer aggression), although this is not strictly necessary, provided the subset of genes is associated with differential expression levels across cancers (such as prostate cancers).

The actual genes on which the analysis is conducted will depend on the expression level information that is available, and it may vary from dataset to dataset. It is not necessary for this method step to be limited to a specific list of genes. However, the genes listed in Table 1 can be used.

Thus, the method of the invention may include the determination of expression status of a much larger number of genes that is needed for the rest of the method. The method may therefore further comprise a step of selecting, from the expression profile for the patient sample, a subset of genes whose expression level is known to vary across prostate cancers. Said subset may be the at least 20, at least 50, at least 100, at least 200 or at least 500 genes selected from Table 1. As noted, the genes are the same genes used in the LPD analysis to provide the reference variables.

Normalisation

Preparation of the reference datasets will generally not be part of the method, since reference datasets are available to the skilled person. When using a previously obtained reference dataset (or even a reference dataset obtained de novo), normalisation of the levels of expression for the plurality of genes in the patient sample to the reference dataset may be required to ensure the information obtained for the patient sample is comparable with the reference dataset. Normalisation techniques are known to the skilled person, for example, Robust Multi-Array Average, Froze Robust Multi-Array Average or Probe Logarithmic Intensity Error when complete microarray datasets are available. Quantile normalisation can also be used. Normalisation may occur after the first expression profile has been combined with the reference dataset to provide a combined dataset that is then normalised.

Methods of normalisation generally involve correction of the measured levels to account for, for example, differences in the amount of RNA assayed, variability in the quality of the RNA used, etc, to put all the genes being analysed on a comparable scale.

In one embodiment of the invention, the method of any preceding claim, wherein the method comprises normalising the patient expression profile to the expression profiles of the reference dataset prior to classifying the cancer.

Methods of Measuring Gene Expression Status

Determining the expression status of a gene may comprise determining the level of expression of the gene. Therefore, references to “expression status” herein also refer to the level of expression of the relevant gene or genes. Expression status and levels of expression as used herein can be determined by methods known the skilled person. For example, this may refer to the up or down-regulation of a particular gene or genes, as determined by methods known to a skilled person. Epigenetic modifications may be used as an indicator of expression, for example determining DNA methylation status, or other epigenetic changes such as histone marking, RNA changes or conformation changes. Epigenetic modifications regulate expression of genes in DNA and can influence efficacy of medical treatments among patients. Aberrant epigenetic changes are associated with many diseases such as, for example, cancer. DNA methylation in animals influences dosage compensation, imprinting, and genome stability and development. Methods of determining DNA methylation are known to the skilled person (for example methylation-specific PCR, matrix-assisted laser desorption/ionization time-of-flight mass spectrometry, use of microarrays, reduced representation bisulfate sequencing (RRBS) or whole genome shotgun bisulfate sequencing (WGBS). In addition, epigenetic changes may include changes in conformation of chromatin.

The expression status of a gene may also be judged examining epigenetic features. Modification of cytosine in DNA by, for example, methylation can be associated with alterations in gene expression. Other way of assessing epigenetic changes include examination of histone modifications (marking) and associated genes, examination of non-coding RNAs and analysis of chromatin conformation. Examples of technologies that can be used to examine epigenetic status are provided in the following publications: Zhang, G. & Pradhan, S. Mammalian epigenetic mechanisms. IUBMB life (2014); Grønœk, K. et al. A critical appraisal of tools available for monitoring epigenetic changes in clinical samples from patients with myeloid malignancies. Haematologica 97, 1380-1388 (2012); Ulahannan, N. & Greally, J. M. Genome-wide assays that identify and quantify modified cytosines in human disease studies. Epigenetics Chromatin 8, 5 (2015); Crutchley, J. L., Wang, X., Ferraiuolo, M. A. & Dostie, J. Chromatin conformation signatures: ideal human disease biomarkers? Biomarkers (2010); and Esteller, M. Cancer epigenomics: DNA methylomes and histone-modification maps. Nat. Rev. Genet. 8, 286-298 (2007).

The methods of the invention may comprise simply providing the expression status (for example the level of expression) of the genes in the patient expression profile, or the method may comprise a step of determining the expression status (for example the level of expression) of the genes in the patient expression profile. The step of determining the level of expression of a plurality of genes in the patient sample can be done by any suitable means known to a person of skill in the art, such as those discussed elsewhere herein, or methods as discussed in any of Prokopec S D, Watson J D, Waggott D M, Smith A B, Wu A H, Okey A B et al. Systematic evaluation of medium-throughput mRNA abundance platforms. RNA 2013; 19: 51-62; Chatterjee A, Leichter A L, Fan V, Tsai P, Purcell R V, Sullivan M J et al. A cross comparison of technologies for the detection of microRNAs in clinical FFPE samples of hepatoblastoma patients. Sci Rep 2015; 5: 10438; Pollock J D. Gene expression profiling: methodological challenges, results, and prospects for addiction research. Chem Phys Lipids 2002; 121: 241-256; Mantione K J, Kream R M, Kuzelova H, Ptacek R, Raboch J, Samuel J M et al. Comparing bioinformatic gene expression profiling methods: microarray and RNA-Seq. Med Sci Monit Basic Res 2014; 20: 138-142; Casassola A, Brammer S P, Chaves M S, Ant J. Gene expression: A review on methods for the study of defense-related gene differential expression in plants. American Journal of Plant Research 2013; 4, 64-73; Ozsolak F, Milos P M. RNA sequencing: advances, challenges and opportunities. Nat Rev Genet 2011; 12: 87-98.

In embodiments of the invention, the patient expression profile is provided as an RNA expression profile or a cDNA expression profile

Methods as described herein that refer to “determining the expression status” or the like include methods in which the expression status (such as quantitative level of expression) is provided, i.e. the expression status has been determined previously and the step of actually determining the expression status is not an explicit step in the method.

The methods steps of the present invention are carried out using the expression status (for example level of expression) of the selected genes. Normalisation and/or comparison to control genes may be conducted as described herein prior to conducting an analysis, as deemed necessary by the skilled person. Similarly, the patient expression profile that is undergoing testing or classification, the patient expression profile comprises the expression status (for example level of expression) of a selection of genes, and the analysis is done using the expression status of those genes from the patient expression profile.

Reference Parameters

The reference parameters determined in a prior step of LPD analysis conducted on a reference dataset are used as a representative framework for the entire cancer population. In particular, the reference parameters define a representative gene expression profile for each cancer expression signature K.

In some embodiments, the reference parameters may be as follows:
a) α—a variable that specifies a Dirichlet distribution in K dimensions, where K is the number of cancer expression signatures;
b) μ—a set of G by K variables, denoted μ_{gk}, storing the means of G×K Gaussian components; and
c) σ—a set of G by K variables, denoted σ_{gk}, storing the variances of G×K Gaussian components, wherein each pair μ_{gk}, σ_{gk} defines the normal distribution that encodes the distribution of expression levels of a given gene in a given cancer signature K

For example, when G is 500 and K is 8, there are 4000μ and 4000 σ values in that set of reference variables. α may be considered as defining the probability of occurrence of each cancer signature in the reference dataset. For example, α may define the probably of co-occurrence of each cancer signature in the reference dataset. It may be considered that the reference parameters define a representative gene expression profile for each cancer expression signature.

Essentially, the reference parameters define or capture a model of the global occurrence of the different cancer expression signatures. The model is built using LPD on a reference dataset, and, on the assumption that the reference dataset provided sufficient information, the reference dataset and resulting reference parameter are used as a model that can be applied to any patient sample. The assumption behind the model is the reference dataset is representative of the entire population.

As the number of genes (and hence G) increases, the accuracy of the classification may increase. Therefore, the number of genes used does not have to be fixed. The present inventors found a good result using 500 different genes, although a smaller (or larger) number of genes could be used. Of course, the same genes are used from each expression profile in the reference dataset. For example, if the dataset comprises 100 expression profiles and the analysis uses 500 genes, the same 500 genes will be selected from each of the 100 expression profiles. Therefore, the analysis will be conducted using 50000 data points (the expression status of the same 500 genes from 100 expression profiles from the reference dataset).

The above reference parameters are derived from the known LPD analysis methods, as described in Rogers et al., 2005, and with which the skilled person is familiar. The new method employed for the first time by the present inventors applies the reference parameters to classify the patient sample(s) in a method referred to herein as OAS-LPD (which does not include the prior steps of determining the reference variables).

The reference parameters are provided by the LPD decomposition method. The decomposition of the reference dataset into 8 groups therefore provides the reference parameters. The reference parameters provided by the LPD decomposition on a reference dataset can be used in an LPD analysis of a patient expression profile. The LPD analysis of the patient expression profile does not comprise devising the reference parameters (a, p and a). Rather, the reference parameters are inputted into the LPD model that is used to analyse the patient expression profile.

The step of determining the contribution of each of the K different cancer expression signatures to the patient expression profile may be achieved by applying the set of reference parameters to the patient expression profile. The classification method is the LPD classification method. The reference parameters are derived by application of LPD to a reference dataset, as described herein. Application of the reference parameters to the patient expression profile is achieved mathematically, for example as described below.

Use of the reference parameters (which define the 8 different cancer expression signatures) allows the patient expression profile to be split (or “decomposed”) into the constituent cancer expression signatures that make up the patient expression profile. It can be considered that the reference parameters split the patient expression profile to provide an optimal weighted combination of the different cancer expression signatures. The weighted combination of the different cancer expression signatures between them make up (i.e. constitute) the patient expression profile. Accordingly, the contribution of each of the 8 different cancer expression signatures to the patient expression profile can be determined. In some cases, there may be some cancer expression signatures that do not contribute at all to the patient expression profile.

The 8 prostate cancer expression signatures represent 8 cancer populations or types that between them represent all types of prostate cancer.

The LPD Method and Implementation of the Reference Variables

The entire LPD method uses the following variables:
1. α—a K-dimensional variable which specifies a Dirichlet distribution, where K is the number of processes. It encodes the dataset-level distribution of processes;
2. θ—a set of A K-dimensional compositional vectors (vectors with K components containing values between 0 and 1, which sum up to 1), denoted θₐ, with 1≤a≤A, where A is the number of samples. Each θₐ vector encodes the weights associated with the K processes, in sample a;
3. e—a set of G by A variables, denoted e_{ag}, storing the observed expression levels of gene g in sample a, with 1≤g≤G, and 1≤a≤A, where G is the number of genes measured;
4. μ—a set of G by K variables, denoted μ_{gk}, storing the means of G×K Gaussian components, with 1≤g≤G, and 1≤k≤K.
5. σ—a set of G by K variables, denoted σ_{gk}, storing the variances of G×K Gaussian components, with 1≤g≤G, and 1≤k≤K. Each pair μ_{gk}, σ_{gk}, defines the normal distribution which encodes the distribution of expression levels of gene g in process k;
6. σ_{μ}—a variable encoding the prior for the μ parameters described at point 4;
7. s—a variable encoding the prior for the σ parameters described at point 5;

In addition to the seven sets of variables which make up the model, the model may also have associated two or more sets of parameters, that can be used during the learning phase as intermediaries to help estimate the values of the model variables described above:
1. Q—a set of K by G by A, variables, denoted Q_{kga}, with 1≤k≤K, 1≤g≤G and 1≤a≤A, which roughly encode the contribution of process k to generating the observed expression level of gene g in sample a.
2. γ—a set of A K-dimensional compositional vectors, denoted γₐ, with 1≤a≤A, approximating the values of variables θₐ. They encode the inferred contribution of each process k to the observed expression profile of sample a.

However, the auxiliary set of variables Q and γ, may be present only if the parameter learning procedure based on variational inference (also called variational Bayes) framework is used for fitting the models. They are not essential to the structure or functioning of the LPD model. If other parameter learning procedures are employed to estimate the values of the models, such as Monte-Carlo methods or other parameter approximation techniques, they might not be present at all, or be present in other forms. Nonetheless, irrespective of the presence of these variables, or the form in which they appear, the structure and functionality of the LPD model remains the same.

The OAS-LPD classification procedure is made up of two stages:
1. The use of standard LPD algorithm on a training set of samples to learn the reference (or model) parameters;
2. The use of a modified procedure, specific to OAS-LPD model, to classify a new sample or a set of new samples. The modified procedure uses the reference parameters derived in step 1.

Stage 1 is identical to a standard LPD learning procedure on a given set of A samples, G genes (which can be 500 or other number) and K processes. Once the stage 1 is finished, the sets of variables α, μ and σ are saved and stored for use in stage 2.

In stage 2, in order to classify a new set of A′ samples, where A′ can be 1 or more patient samples that is/are undergoing classification, the following steps can be followed:
1. A new instance of the OAS-LPD model is created, using A′ samples, and the same set of G genes and K used in stage 1.
2. The sets of variables α, μ and σ are initialised with the values determined at stage 1.
3. The set of variables θ are inferred using a suitable learning procedure. One such procedure can as follows:
   a. Initialise the K components of vector γₐ with random values between 0 and 1, with the constraint that they sum to 1 across the K components;
   b. For a number of maxlterations iterations (where maxlterations is a positive natural number chosen by a skilled person), do:
i. Using α, μ and σ as provided as the reference variables, calculate Q_{kga} as in the following equation:

$${Q}_{kga} = \frac{𝒩 ( {e}_{ga} \text{\textbar} k , {μ}_{gk} , {σ}_{gk} ) exp [ ψ ( {γ}_{ak} ) ]}{{\sum }_{K = 1}^{K} 𝒩 ( {e}_{ga} \text{\textbar} k , {μ}_{gk} , {σ}_{gk} ) exp [ ψ ( {γ}_{ak} ) ]} ,$$

ii. Calculate γₐₖ as in the following equation, using a as provided as the reference variables and Q_{kga} as calculated at step (b)(i):

$${γ}_{ak} = {α}_{k} + {\sum }_{g = 1}^{ℊ} {Q}_{kga} ,$$

When the algorithm finishes, variables γ contain approximations for parameters θ, which encode the OAS-LPD classification of each A′ sample. θ values are the ideal weighted combination of the gene signatures to give the sample expression profile. Thus, these equations determine the make-up of a patient's cancer as defined by the cancer gene signatures. For each sample, the analysis provides K outputs, i.e. one θₐ set of values (represented by its approximation γₐ) for each patient expression profile that is being analysed, as is clear from the above notation γₐₖ where γ is provided for each k (cancer gene signature) of each a (patient expression profile).

Accordingly, in some embodiments, the patient's cancer is classified by inputting the patient expression profile (i.e. the expression status of the selected genes) and reference parameters into equations (i) and (ii) above.

Further details are provided in the Examples section below.

Contribution of the Cancer Gene Expression Signature to the Patient Gene Expression Profile

As noted above, the methods comprise determining the contribution of each different cancer gene expression signature to the patient gene expression profile. The contribution of each signature to the patient expression profile may be denoted pᵢ (note pᵢ is also referred to herein as gamma (γ), and both are an approximation of θ, as defined in the formulae above). The present inventors have shown that pᵢ is a continuous variable (as opposed to a discrete variable) and is a measure of the contribution of a given signature to the expression profile of a given sample. The higher the contribution of a given signature (so the higher the value of pᵢ for the signature contributing to the expression profile for a given sample), the greater the chance the cancer will exhibit the features of the cancer associated with that cancer expression signature. For example, if we consider one cancer expression signature that is associated with poor prognosis (for example the cancer population referred to as DESNT or S7 herein) then the larger the value of pᵢ the worse the outcome will be.

For a given sample, a number of different signatures can contribute to an expression profile. For example it is not always necessary for the DESNT signature to be the most dominant (i.e. to have to highest pᵢ value of all the processes contributing to the expression profile) for a poor outcome to be predicted. However, the higher the pᵢ value for a poor prognosis cancer the worse the patient outcome; not only in reference to PSA failure but also metastasis and death are also more likely. In some embodiments, the contribution of a cancer class associated with a particular prognosis (such as a poor prognosis, as for the DESNT signature, or a good prognosis) to the overall expression profile for a given cancer may be determined when assessing the likelihood of a cancer progressing. In some embodiments, the prediction of cancer progression may be done by reference to the cancer classification as determined according to a method of the invention, and further in combination with one or more of stage of the tumour, Gleason score and/or PSA score. Therefore, in some embodiments, the step of determining the cancer prognosis may comprise a step of determining the pᵢ value for a signature associated with a poor outcome for the patient expression profile (i.e. the contribution of the signature associated with a poor outcome to the overall patient expression profile), for example the DESNT signature, and, optionally, further determining the stage of the tumour, the Gleason score of the patient and/or PSA score of the patient.

In some embodiments, the step of classifying the cancer in the sample from the patient comprises, for each expression profile being tested, using the method to determine the contribution (pᵢ) of each signature K to the overall expression profile (wherein the sum of all pᵢ values for a given patient expression profile is 1). The patient expression profile may be assigned to an individual group according to the group that contributes the most to the overall expression profile (in other words, the patient expression profile is assigned to the group with the highest pᵢ value). In some embodiments, each signature is assigned either as a poor prognosis signature or a good prognosis signature. Cancer progression in the patient can be predicted according to the contribution (pᵢ value) of the different signatures to the overall expression profile. In some embodiments, poor prognosis cancer is predicted when the pᵢ value for a poor prognosis signature (such as DESNT) for the patient cancer sample is at least 0.1, at least 0.2, at least 0.3, at least 0.4 or at least 0.5.

The contribution of a given cancer signature to a patient expression profile may be informative of the level of sensitivity or resistance to a particular treatment. For example, if a cancer signature is associated with a sensitivity to a particular drug treatment, the higher the contribution of that cancer signature to the patient expression profile, the more sensitive the patient may be to that drug treatment. Conversely, the lower the contribution of that cancer signature to the patient expression profile, the less sensitive (or indeed the more resistant) the patient may be to that drug treatment. Given the contribution of each signature to the overall patient expression profile is a continuous variable, the sensitivity or resistance of a patient to a treatment can be determined.

In one embodiment of the invention, the contribution of each cancer expression signature to the patient expression profile can be expressed as a value between 0 and 1, and wherein the combination of all of the cancer expression signatures contributing to a given patient expression profile is equal to 1. Additionally, the contribution of each cancer expression signature to the patient expression profile is a continuous variable. The contribution of each cancer expression signature to the patient expression profile may determine a property of the cancer. In particular, the amount a specific patient's cancer exhibits a particular property may be determined by the level of contribution of the corresponding cancer expression signature to the patient expression profile. For example, if a cancer expression signature is associated with a poor prognosis, the higher the prevalence of that cancer expression signature to the patient expression profile, the worse the prognosis is for the patient. Similarly, if a cancer expression signature is associated with a drug sensitivity, the higher the prevalence of that cancer expression signature to the patient expression profile the more sensitive that patient may be to the drug treatment.

Accordingly, in one embodiment, one or more of the cancer expression signatures are correlated with one or more properties (such as a cancer prognosis or treatment sensitivity). The level of contribution of a given cancer expression signature to a patient's expression profile determines the degree to which the patient's cancer exhibits the corresponding property.

Cancer Populations Identified Using Methods of the Invention

The present inventors devised the methods using prostate cancer datasets as the reference datasets. The inventors surprisingly found the datasets could be reliably decomposed into 8 different processes (cancer expression signatures) based on the decomposition of 2 different datasets, wherein the decomposition of the 2 datasets resulted in the same 8 processes for both datasets, despite the different input data. Each different signature can be considered a different cancer classification as it is associated with a different cancer population. The different cancer populations are distinguishable from each other according to their gene expression profile, gene mutation profile and/or the clinical outcome of the cancer. The different cancer populations may also be distinguishable from each other according to their drug treatment sensitives (for example susceptibility or resistance to a particular treatment).

Accordingly, in embodiments of the invention, each cancer classification K may be defined according to its gene expression profile, gene mutation profile and/or the clinical outcome of the cancer.

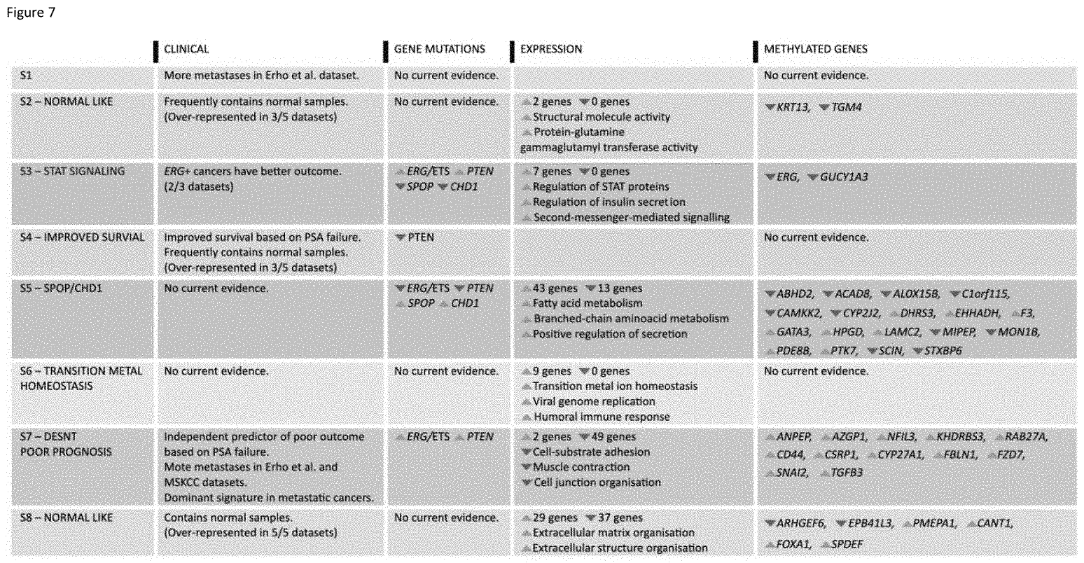

The different prostate cancer populations are referred to herein as S1, S2, S3, S4, S5, S6, S7 and S8. The different populations may be distinguished from each other according to one or more criteria as set out in FIG. 7.

Some of the different cancer populations may be distinguishable from each other according to up and/or down regulation of certain genes, and/or according to a relative increase or decrease of the prevalence of different mutations. The up and/or down regulation of certain genes, and the relative increase or decrease of the prevalence of different mutations are with respect to the other prostate cancer populations.

For example, the S2 prostate cancer population may be associated upregulation of one or more of KRT13 and TGM4.

The S3 prostate cancer population may be associated with upregulation of one or more of CSGALNACT1, ERG, GHR, GUCY1A3, HDAC1, ITPR3 and PLA2G7. For example, in one embodiment, the S3 prostate cancer population may be associated with upregulation of all of CSGALNACT1, ERG, GHR, GUCY1A3, HDAC1, ITPR3 and PLA2G7. The S3 prostate cancer population may be further associated with a increase in the number of mutations in one or more of ERG and PTEN and/or an decrease in the number of mutations in one or more of SPOP and CHD1. ERG positive cancers in this group may be associated with an improved outcome.

The S5 prostate cancer population may be associated with upregulation of one or more of ABHD2, ACAD8, ACLY, ALCAM, ALDH6A1, ALOX15B, ARHGEF7, AUH, BBS4, Clorf115, CAMKK2, COGS, CPEB3, CYP2J2, DHX32, EHHADH, ELOVL2, EXTL2, FAM111A, GLUD1, GNMT, HPGD, MIPEP, MON1B, NANS, NAT1, NCAPD3, PPFIBP2, PTPN13, PTPRM, RAB27A, REPS2, RFX3, SCIN, SLC1A1, SLC4A4, SMPDL3A, STXBP6, SYTL2, TBPL1, TFF3, TUBB2A, and YIPF1 and/or downregulation of one or more of DHRS3, ERG, F3, GATA3, HES1, KHDRBS3, LAMB2, LAMC2, PDE8B, PTK7, SORL1, TRIM29 and ZNF516. For example, in one embodiment, the S5 prostate cancer population may be associated with upregulation of at least 75% of the genes selected from the group consisting of ABHD2, ACAD8, ACLY, ALCAM, ALDH6A1, ALOX15B, ARHGEF7, AUH, BBS4, Clorf115, CAMKK2, COGS, CPEB3, CYP2J2, DHX32, EHHADH, ELOVL2, EXTL2, FAM111A, GLUD1, GNMT, HPGD, MIPEP, MON1B, NANS, NAT1, NCAPD3, PPFIBP2, PTPN13, PTPRM, RAB27A, REPS2, RFX3, SCIN, SLC1A1, SLC4A4, SMPDL3A, STXBP6, SYTL2, TBPL1, TFF3, TUBB2A, and YIPF1 and downregulation of at least 75% of the genes selected from the group consisting of DHRS3, ERG, F3, GATA3, HES1, KHDRBS3, LAMB2, LAMC2, PDE8B, PTK7, SORL1, TRIM29 and ZNF516. In one embodiment, the S5 prostate cancer population may be associated with upregulation of all of ABHD2, ACAD8, ACLY, ALCAM, ALDH6A1, ALOX15B, ARHGEF7, AUH, BBS4, Clorf115, CAMKK2, COGS, CPEB3, CYP2J2, DHX32, EHHADH, ELOVL2, EXTL2, FAM111A, GLUD1, GNMT, HPGD, MIPEP, MON1B, NANS, NAT1, NCAPD3, PPFIBP2, PTPN13, PTPRM, RAB27A, REPS2, RFX3, SCIN, SLC1A1, SLC4A4, SMPDL3A, STXBP6, SYTL2, TBPL1, TFF3, TUBB2A, and YIPF1 and downregulation of all of DHRS3, ERG, F3, GATA3, HES1, KHDRBS3, LAMB2, LAMC2, PDE8B, PTK7, SORL1, TRIM29 and ZNF516.

The S5 prostate cancer population may be further associated with an increase in the number of mutation in one or more of ERG and PTEN and/or a decrease in the number of mutations in one or more of SPOP and CHD1. In one embodiment, the S5 prostate cancer population may be further associated with an increase in the number of mutations in ERG and PTEN and a decrease in the number of mutations of SPOP and CHD1.

The S6 prostate cancer population may be associated with upregulation of one or more of CCL2, CFB, CFTR, CXCL2, 1F116, LCN2, LTF, LXN and TFRC. In one embodiment, the S6 prostate cancer population may be associated with upregulation of at least 75% of the genes selected from the group consisting of CCL2, CFB, CFTR, CXCL2, 1F116, LCN2, LTF, LXN and TFRC. In one embodiment, the S6 prostate cancer population may be associated with upregulation of all of CCL2, CFB, CFTR, CXCL2, 1F116, LCN2, LTF, LXN and TFRC.

The S7 prostate cancer population (also referred to as DESNT herein) may be associated with upregulation of one or more of F5 and KHDRBS3, and downregulation of one or more of ACTG2, ACTN1, ADAMTS1, ANPEP, ARMCX1, AZGP1, C7, CD44, CHRDL1, CNN1, CRISPLD2, CSRP1, CYP27A1, CYR61, DES, EGR1, ETS2, FBLN1, FERMT2, FHL2, FLNA, FXYD6, FZD7, ITGA5, ITM2C, JAM3, JUN, LMOD1, LPHN2, MT1M, MYH11, MYL9, NFIL3, PARM1, PCP4, PDK4, PLAGL1, RAB27A, SERPINF1, SNAI2, SORBS1, SPARCL1, SPOCK3, SYNM, TAGLN, TCEAL2, TGFB3, TPM2 and VCL. In one embodiment, the S7 prostate cancer population may be associated with upregulation F5 and KHDRBS3 and downregulation of at least 75% of the genes selected from the group consisting of ACTG2, ACTN1, ADAMTS1, ANPEP, ARMCX1, AZGP1, C7, CD44, CHRDL1, CNN1, CRISPLD2, CSRP1, CYP27A1, CYR61, DES, EGR1, ETS2, FBLN1, FERMT2, FHL2, FLNA, FXYD6, FZD7, ITGA5, ITM2C, JAM3, JUN, LMOD1, LPHN2, MT1M, MYH11, MYL9, NFIL3, PARM1, PCP4, PDK4, PLAGL1, RAB27A, SERPINF1, SNAI2, SORBS1, SPARCL1, SPOCK3, SYNM, TAGLN, TCEAL2, TGFB3, TPM2 and VCL. In one embodiment, the S7 prostate cancer population may be associated with upregulation of F5 and KHDRBS3 and downregulation of all of ACTG2, ACTN1, ADAMTS1, ANPEP, ARMCX1, AZGP1, C7, CD44, CHRDL1, CNN1, CRISPLD2, CSRP1, CYP27A1, CYR61, DES, EGR1, ETS2, FBLN1, FERMT2, FHL2, FLNA, FXYD6, FZD7, ITGA5, ITM2C, JAM3, JUN, LMOD1, LPHN2, MT1 M, MYH11, MYL9, NFIL3, PARM1, PCP4, PDK4, PLAGL1, RAB27A, SERPINF1, SNAI2, SORBS1, SPARCL1, SPOCK3, SYNM, TAGLN, TCEAL2, TGFB3, TPM2 and VCL.

The S7 prostate cancer population may be further associated with an increase in the number of mutation in one or more of ERG and PTEN.

The S8 prostate cancer population may be associated with upregulation of one or more of ARHGEF6, AXL, CD83, COL15A1, DPYSL3, EPB41L3, FBN1, FCHSD2, FHL1, FXYD5, GNAO1, GPX3, 1F116, IRAK3, ITGA5, LAPTM5, MFAP4, MFGE8, MMP2, PARVA, PLEKHO1, PLSCR4, RFTN1, SAMD4A, SAMSN1, SERPINF1, VCAM1, WIPF1 and ZYX and/or downregulation of one or more of ABCC4, ACAT2, ATP8A1, CANT1, CDH1, DCXR, DHCR24, DHRS7, FAM174B, FAM189A2, FKBP4, FOXA1, GOLM1, GTF3C1, HPN, KIF5C, KLK3, MAP7, MBOAT2, MIOS, MLPH, MYO5C, NEDD4L, PART1, PDIA5, PIGH, PMEPA1, PRSS8, SEC23B, SLC43A1, SPDEF, SPINT2, STEAP4, TMPRSS2, TRPM8, TSPAN1, XBP1. In one embodiment, the S8 prostate cancer population may be associated with upregulation of at least 75% of the genes selected from the group consisting of ARHGEF6, AXL, CD83, COL15A1, DPYSL3, EPB41L3, FBN1, FCHSD2, FHL1, FXYD5, GNAO1, GPX3, 1F116, IRAK3, ITGA5, LAPTM5, MFAP4, MFGE8, MMP2, PARVA, PLEKHO1, PLSCR4, RFTN1, SAMD4A, SAMSN1, SERPINF1, VCAM1, WIPF1 and ZYX and downregulation of at least 75% of the genes selected from the group consisting of ABCC4, ACAT2, ATP8A1, CANT1, CDH1, DCXR, DHCR24, DHRS7, FAM174B, FAM189A2, FKBP4, FOXA1, GOLM1, GTF3C1, HPN, KIF5C, KLK3, MAP7, MBOAT2, MIOS, MLPH, MYO5C, NEDD4L, PART1, PDIA5, PIGH, PMEPA1, PRSS8, SEC23B, SLC43A1, SPDEF, SPINT2, STEAP4, TMPRSS2, TRPM8, TSPAN1, XBP1. In one embodiment, the S8 prostate cancer population may be associated with upregulation of all of ARHGEF6, AXL, CD83, COL15A1, DPYSL3, EPB41L3, FBN1, FCHSD2, FHL1, FXYD5, GNAO1, GPX3, IF116, IRAK3, ITGA5, LAPTM5, MFAP4, MFGE8, MMP2, PARVA, PLEKHO1, PLSCR4, RFTN1, SAMD4A, SAMSN1, SERPINF1, VCAM1, WIPF1 and ZYX and downregulation of all of ABCC4, ACAT2, ATP8A1, CANT1, CDH1, DCXR, DHCR24, DHRS7, FAM174B, FAM189A2, FKBP4, FOXA1, GOLM1, GTF3C1, HPN, KIF5C, KLK3, MAP7, MBOAT2, MIOS, MLPH, MYO5C, NEDD4L, PART1, PDIA5, PIGH, PMEPA1, PRSS8, SEC23B, SLC43A1, SPDEF, SPINT2, STEAP4, TMPRSS2, TRPM8, TSPAN1, XBP1.

In the context of cancer classifications being “associated with” upregulation and/or down regulation of certain genes, this refers to a patient example belonging to a given cancer classification exhibiting the upregulation and/or down regulation of the specified genes. In some embodiments, this may be upregulation and/or down regulation of the specified genes compared to a one or house-keeping genes or a healthy control (no prostate cancer present). In some embodiments, this may be upregulation and/or down regulation with respect to other cancer classifications.

As noted above, the different cancer classes or populations may be associated with different clinical outcomes. Accordingly, in some embodiments, one or more of the cancer classifications are associated with a cancer prognosis. In one embodiment of the invention, the cancer is prostate cancer and K is 7, 8 or 9, and wherein at least one of the prostate cancer classifications is associated with a poor prognosis. Other values of K could be used, although some of the same cancer populations may still be identified. In preferred embodiments, K is 8.

The S7 cancer population is associated with a poor prognosis. This cancer signature may also be referred to herein as DESNT cancer. As used herein, “DESNT” cancer refers to prostate cancer with a poor prognosis and one that requires treatment. “DESNT status” refers to whether or not the cancer is predicted to progress (or, for historical data, has progressed), hence a step of determining DESNT status refers to predicting whether or not a cancer will progress and hence require treatment. Progression may refer to elevated PSA, metastasis and/or patient death. The present invention is useful in identifying patients with a potentially poor prognosis and recommending them for treatment. If a cancer is not assigned to the S7 group, it may be referred to as a “non-DESNT cancer”. Predictions of clinical outcome can be made if the patient expression profile is assigned to the S7 cancer population.

In one embodiment of the invention, the cancer is prostate cancer and K is 7, 8 or 9, and at least one of the prostate cancer classifications is associated with a good prognosis. The S4 cancer population identified by the present inventors is consistently associated with a good clinical outcome and therefore a good prognosis. Predictions of clinical outcome can also be made if the patient expression profile is assigned to the S4 cancer population.

In a cancer signature is not associated with any particular gene expression profile, gene mutation profile and/or clinical outcome of the cancer, the cancer population may be the S1 cancer population as defined herein.

Accordingly, in some embodiments, the methods may comprise predicting an increased likelihood of cancer progression. Such a prediction may be made if the cancer is prostate cancer and is classified as the S7 cancer population. Accordingly, in some embodiments, the methods may comprise predicting a decreased likelihood of cancer progression. Such a prediction may be made if the cancer is prostate cancer and is classified as the S4 cancer population.

Any of the methods of the invention may be carried out in patients in whom a cancer, in particular an aggressive cancer, is suspected. Importantly, the present invention allows a prediction of cancer progression before treatment of cancer is provided. This is particularly important for prostate cancer, since many patients will undergo unnecessary treatment for prostate cancer when the cancer would not have progressed even without treatment. The present invention also allows prediction of a patient's suitability for a drug treatment according to the suitability of the assigned cancer signature to said drug treatment.

Each cancer population identified by the present inventors may be considered a continuous variable.

In some embodiments of the invention, the methods may comprise determining the contribution of each of the cancer populations to the patient expression profile and assigning the cancer to a cancer population according to the cancer population that contributes the most to the patient expression profile. A suitable course of action regarding therapy or intervention in the cancer can therefore be taken.

Random Forest and LASSO Methods of the Invention

The presents inventors wished to develop an alternative classifier that did not require the use of the LPD or the use of the LPD reference variables. The following methods provide such a solution.

Supervised machine learning algorithms or general linear models can be used to produce a predictor cancer classification. The preferred approach is random forest analysis but alternatives such as support vector machines, neural networks, naive Bayes classifier, or nearest neighbour algorithms could be used. Such methods are known and understood by the skilled person.

In one embodiment of the invention, there is provided a method of classifying cancer or predicting cancer progression, comprising:
a) providing one or more reference datasets where the cancer classification of each patient sample in the datasets is known (for example as determined by LPD analysis);
b) selecting from this dataset a plurality of genes;
c) applying a LASSO logistic regression model analysis on the selected genes to identify a subset of the selected genes that are predictive of each cancer classification;
d) using the expression status of this subset of selected genes to apply a supervised machine learning algorithm on the dataset to obtain a predictor for each cancer classification;
e) providing or determining the expression status of the subset of selected genes in a sample obtained from the patient to provide a patient expression profile;
f) optionally normalising the patient expression profile to the reference dataset(s); and
g) applying the predictor to the patient expression profile to classify the cancer or predict cancer progression.

Such a method may be referred to herein as Method 2.

Preferably, the genes selected in step (b) are known to vary between cancer classifications (i.e. they vary across at least 2 of the cancer classifications). However, virtually any genes can be selected in step (b). The same genes are used from each patient sample as used in the patient samples from the reference dataset. In some embodiments, at least 10,000 different genes are selected in step (b). In one embodiment, the plurality of genes selected in step (b) comprises at least 1000, at least 5000, or at least different 10,000 genes from the human genome. The same genes are selected from each expression profile in the dataset. Application of a LASSO analysis to the selected genes refers to application of a LASSO analysis to the expression status (for example level of expression) of the selected genes.

The analysis step (c) is conducted on the expression status data (for example level of gene expression) for each gene selected in step (b).

The above method includes a step of identifying genes that are informative of the cancer signatures that may be present in a patient sample. However, it is not always necessary to include the step of determining the genes that are informative. For example, one of the contributions of the present invention is the identification of the genes that are informative for the different prostate cancer classification. The present inventors have used the LASSO method to identify the 203 genes of Table 2 that are informative as to the contribution of each cancer expression signature to a patient's cancer.

For example, in one embodiment of the invention, there is provided a method of classifying cancer or predicting cancer progression, comprising:
a) providing one or more reference datasets where the cancer classification of each patient sample in the datasets is known (for example as determined by LPD analysis);
b) selecting from this dataset a plurality of genes, wherein the plurality of genes comprises at least 5, at least 10, at least 20, at least 30, at least 40, at least 50, at least 100, or at least 150 genes selected from the group listed in Table 2
c) optionally:
   i. determining the expression status of at least 1 further, different, gene in the patient sample as a control, wherein the control gene is not a gene listed in Table 2; and
   ii. determining the relative levels of expression of the plurality of genes and of the control gene(s);
d) using the expression status of those selected genes to apply a supervised machine learning algorithm on the dataset to obtain a predictor for each cancer classification;
e) determining or providing the expression status of the same plurality of genes in a sample obtained from the patient to provide a patient expression profile;
f) optionally normalising the patient expression profile to the reference dataset; and
g) applying the predictor to the patient expression profile to classify the cancer, or to predict cancer progression.

Such a method may be referred to herein as Method 3. The genes of Table 2 were identified by the inventors by conducting a LASSO analysis as described in Method 2.

In a preferred embodiment, the control genes used in step (i) are selected from the housekeeping genes listed in Table 3 or Table 4. Table 4 is particularly relevant to prostate cancer. In some embodiments of the invention, at least 1, at least 2, at least 5 or at least 10 housekeeping genes. Preferred embodiments use at least 2 housekeeping genes. Step (ii) above may comprise determining a ratio between the test genes and the housekeeping genes.

Alternatively, there is provided a method of classifying cancer or predicting cancer progression, comprising:
a) providing a reference dataset wherein the cancer classification of each patient sample in the dataset is known (for example as determined by LPD analysis);
b) selecting from this dataset of a plurality of genes;
c) using the expression status of those selected genes to apply a supervised machine learning algorithm on the dataset to obtain a predictor for cancer classification;
d) providing or determining the expression status of the same plurality of genes in a sample obtained from the patient to provide a patient expression profile;
e) optionally normalising the patient expression profile to the reference dataset; and
f) applying the predictor to the patient expression profile to classify the cancer, or to predict cancer progression.

Such a method may be referred to herein as Method 4. The genes selected in step (b) preferably are known to vary between cancer classifications (i.e. they vary across at least 2 of the cancer classifications). However, virtually any genes can be selected in step (b). The same genes are used from each patient sample as used in the patient samples from the reference dataset. In some embodiments, at least 500 genes are selected in step (b). In one embodiment, the plurality of genes selected in step (b) comprises at least 100, at least 200, or at least 500 genes from the human genome.

In methods such as the three Methods 2 to 4 of the invention described above, when the cancer is prostate cancer, each patient sample in the dataset may be assigned to one of the 51 to S8 populations. In one embodiment, step a) comprises providing one or more reference datasets where the contribution of each of the 51 to S8 cancer classifications to each patient sample in the datasets is known. Each patient sample in the dataset may be further assigned a cancer population according to the population that contributes the most to the patient expression profile.

Such determination may be made by performing an LPD analysis on the reference dataset. In particular, the method may comprise performing an LPD analysis on the reference dataset using a K of 8, since the present inventors have determined the existence of 8 prostate cancer populations that is common across at least 2 reference datasets, and hence is used as a framework for the global occurrence of prostate cancer in humans.

Supervised machine learning algorithms or general linear models are used to produce a predictor of cancer classification. The preferred approach is random forest analysis but alternatives such as support vector machines, neural networks, naive Bayes classifier, or nearest neighbour algorithms could be used. Such methods are known and understood by the skilled person.

The supervised machine learning algorithm used in the above methods is preferably random forest.

Random forest analysis can be used to predict cancer classification. A random forest analysis is an ensemble learning method for classification, regression and other tasks, which operates by constructing a multitude of decision trees during training and outputting the class that is the mode of the classes (classification) or mean prediction (regression) of the individual decision trees. Accordingly, a random forest corrects for overfitting of data to any one decision tree.

A decision tree comprises a tree-like graph or model of decisions and their possible consequences, including chance event outcomes. Each internal node of a decision tree typically represents a test on an attribute or multiple attributes (for example whether an expression level of a gene in a cancer sample is above a predetermined threshold), each branch of a decision tree typically represents an outcome of a test, and each leaf node of the decision tree typically represents a class (classification) label.

In a random forest analysis, an ensemble classifier is typically trained on a training dataset (also referred to as a reference dataset) where the cancer classification for each sample in the dataset, for example as determined by LPD, is known. The training produces a model that is a predictor for membership of the different cancer classifications. Once trained the random forest classifier can then be applied to a dataset from an unknown sample. This step is deterministic i.e. if the classifier is subsequently applied to the same dataset repeatedly, it will consistently sort each cancer of the new dataset into the same class each time.

The ensemble classifier acts to classify each cancer sample in the new dataset into the different cancer classifications. Accordingly, when the random forest analysis is undertaken, the ensemble classifier splits the cancers in the dataset being analysed into a number of classes. The number of classes may be 2 (i.e. the ensemble classifier may group or classify the patients in the dataset into a DESNT class, or DESNT group, containing the DESNT cancers and a non-DESNT class, or non-DESNT group, containing other cancers), or preferably for prostate cancer, the number of classes may be 8 representing cancer populations S1 to S8.

Each decision tree in the random forest is an independent predictor that, given a cancer sample, assigns it to one of the classes which it has been trained to recognize. Each node of each decision tree comprises a test concerning one or more genes of the same plurality of genes as obtained in the cancer sample from the patient. Several genes may be tested at the node. For example, a test may ask whether the expression level(s) of one or more genes of the plurality of genes is above a predetermined threshold.

Variations between decision trees will lead to each decision tree assigning a sample to a class in a different way. The ensemble classifier takes the classification produced by all the independent decision trees and assigns the sample to the class on which the most decision trees agree.

The provision of the plurality of genes for which the level of expression is determined in step b) of Method 3 was achieved by performing a least absolute shrinkage and selection operator (LASSO) analysis on a training dataset and to select those genes that are found to best characterise the different cancer classifications (as exemplified in Method 2). A logistic regression model is derived with a constraint on the coefficients such that the sum of the absolute value of the model coefficients is less than some threshold. This has the effect of removing genes that either don't have the ability to predict cancer classification or are correlated with the expression of a gene already in the model. LASSO is a mathematical way of finding the genes that are most likely to distinguish cancer classifications of the samples from each other in a training or reference dataset.

When devising Method 3, a LASSO logistic regression model was used to predict cancer classification in a reference dataset leading to the selection of a set of 203 genes that characterized the 8 different cancer classifications. These genes are listed in Table 2. Additional sets of genes could be obtained by carrying out the same analyses using other datasets that have been analysed by LPD as a starting point.

Biomarker Panels

The invention therefore provides further lists of genes that are associated with or predictive of cancer classifications and hence are associated with or predictive of cancer progression. For example, in one embodiment, a LASSO analysis can be used to provide an expression signature that is indicative or predictive of cancer classification, in particular prostate cancer classification. The predictive genes may also be considered a biomarker panel, and may comprise at least 5, at least 10, at least 20, at least 30, at least 40, at least 50, at least 100, or at least 150 genes selected from the group listed in Table 2. In some embodiments, this biomarker panel comprises all of the genes selected from Table 2. However, a different set of equally informative genes could be generated using Method 2 of the present invention.

Thus, the methods of the invention provide methods of classifying cancer, some methods comprising determining the expression level or expression status of a one or members of a biomarker panel. The panel of genes may be determined using a method of the invention. In some embodiments, the panel of genes may comprise at least 5, at least 10, at least 20, at least 30, at least 40, at least 50, at least 100, or at least 150 genes selected from the group listed in Table 2.

Other biomarker panels of the invention, or those generated using methods of the invention, may also be used. For example, the present invention also provides biomarker panels useful in defining the prostate cancer classifications identified by the present inventors.

For example, the following biomarker panels are provided:

Biomarker panel A (based on cancer population S2):
KRT13 and TGM4.

In one embodiment of the invention, upregulation of the genes of biomarker panel A may be indicative of the presence of the S2 prostate cancer. Cancers of this type may be a good prognosis. However, analysis in combination with other markers for prostate cancer (such as Gleason score, PSA etc.) may bed done for further confirmation.

Biomarker panel B (based on cancer population S3):
CSGALNACT1, ERG, GHR, GUCY1A3, HDAC1, ITPR3 and PLA2G7

In one embodiment of the invention, upregulation of at least 75% of the genes of biomarker panel B (for example all of the genes in biomarker panel B) may be indicative of the presence of the S3 prostate cancer. When this cancer population are also ERG positive cancers, the prognosis may be good. However, analysis in combination with other markers for prostate cancer (such as Gleason score, PSA etc.) may be done for further confirmation.

Biomarker panel C (based on cancer population S5):
ABHD2, ACAD8, ACLY, ALCAM, ALDH6A1, ALOX15B, ARHGEF7, AUH, BBS4, Clorf115, CAMKK2, COGS, CPEB3, CYP2J2, DHX32, EHHADH, ELOVL2, EXTL2, FAM111A, GLUD1, GNMT, HPGD, MIPEP, MON1B, NANS, NAT1, NCAPD3, PPFIBP2, PTPN13, PTPRM, RAB27A, REPS2, RFX3, SCIN, SLC1A1, SLC4A4, SMPDL3A, STXBP6, SYTL2, TBPL1, TFF3, TUBB2A, YIPF1, DHRS3, ERG, F3, GATA3, HES1, KHDRBS3, LAMB2, LAMC2, PDE8B, PTK7, SORL1, TRIM29 and ZNF516.

In one embodiment of the invention, upregulation of at least 75% of genes selected from the group consisting of ABHD2, ACAD8, ACLY, ALCAM, ALDH6A1, ALOX15B, ARHGEF7, AUH, BBS4, Clorf115, CAMKK2, COGS, CPEB3, CYP2J2, DHX32, EHHADH, ELOVL2, EXTL2, FAM111A, GLUD1, GNMT, HPGD, MIPEP, MON1B, NANS, NAT1, NCAPD3, PPFIBP2, PTPN13, PTPRM, RAB27A, REPS2, RFX3, SCIN, SLC1A1, SLC4A4, SMPDL3A, STXBP6, SYTL2, TBPL1, TFF3, TUBB2A, and YIPF1 (for example upregulation of all of the genes in that group) and downregulation of at least 75% of genes selected from the group consisting of DHRS3, ERG, F3, GATA3, HES1, KHDRBS3, LAMB2, LAMC2, PDE8B, PTK7, SORL1, TRIM29 and ZNF516 (for example upregulation of all of the genes in that group) may be associated with the S5 cancer population.

Biomarker panel D (based on cancer population S6):
CCL2, CFB, CFTR, CXCL2, 1F116, LCN2, LTF, LXN and TFRC.

In one embodiment of the invention, upregulation of at least 75% of genes of biomarker panel D (for example upregulation of all of the genes in that group) may be associated with the S6 cancer population.

Biomarker panel E (based on cancer population S7):
F5, KHDRBS3, ACTG2, ACTN1, ADAMTS1, ANPEP, ARMCX1, AZGP1, C7, CD44, CHRDL1, CNN1, CRISPLD2, CSRP1, CYP27A1, CYR61, DES, EGR1, ETS2, FBLN1, FERMT2, FHL2, FLNA, FXYD6, FZD7, ITGA5, ITM2C, JAM3, JUN, LMOD1, LPHN2, MT1M, MYH11, MYL9, NFIL3, PARM1, PCP4, PDK4, PLAGL1, RAB27A, SERPINF1, SNAI2, SORBS1, SPARCL1, SPOCK3, SYNM, TAGLN, TCEAL2, TGFB3, TPM2 and VCL

In one embodiment of the invention, upregulation of F5 and KHDRBS3 and downregulation of at least 75% of genes selected from the group consisting of ACTG2, ACTN1, ADAMTS1, ANPEP, ARMCX1, AZGP1, C7, CD44, CHRDL1, CNN1, CRISPLD2, CSRP1, CYP27A1, CYR61, DES, EGR1, ETS2, FBLN1, FERMT2, FHL2, FLNA, FXYD6, FZD7, ITGA5, ITM2C, JAM3, JUN, LMOD1, LPHN2, MT1M, MYH11, MYL9, NFIL3, PARM1, PCP4, PDK4, PLAGL1, RAB27A, SERPINF1, SNAI2, SORBS1, SPARCL1, SPOCK3, SYNM, TAGLN, TCEAL2, TGFB3, TPM2 and VCL (for example upregulation of all of the genes in that group) may be associated with the S7 cancer population. Such cancer populations may be associated with a poor prognosis. However, analysis in combination with other markers for prostate cancer (such as Gleason score, PSA etc.) may be done for further confirmation.

Biomarker panel F (based on cancer population S8)
ARHGEF6, AXL, CD83, COL15A1, DPYSL3, EPB41L3, FBN1, FCHSD2, FHL1, FXYD5, GNAO1, GPX3, 1F116, IRAK3, ITGA5, LAPTM5, MFAP4, MFGE8, MMP2, PARVA, PLEKHO1, PLSCR4, RFTN1, SAMD4A, SAMSN1, SERPINF1, VCAM1, WIPF1 and ZYX and/or downregulation of one or more of ABCC4, ACAT2, ATP8A1, CANT1, CDH1, DCXR, DHCR24, DHRS7, FAM174B, FAM189A2, FKBP4, FOXA1, GOLM1, GTF3C1, HPN, KIF5C, KLK3, MAP7, MBOAT2, MIOS, MLPH, MY05C, NEDD4L, PART1, PDIA5, PIGH, PMEPA1, PRSS8, SEC23B, SLC43A1, SPDEF, SPINT2, STEAP4, TMPRSS2, TRPM8, TSPAN1, XBP1.

In one embodiment of the invention, upregulation of at least 75% of genes selected from the group consisting of ARHGEF6, AXL, CD83, COL15A1, DPYSL3, EPB41L3, FBN1, FCHSD2, FHL1, FXYD5, GNAO1, GPX3, 1F116, IRAK3, ITGA5, LAPTM5, MFAP4, MFGE8, MMP2, PARVA, PLEKHO1, PLSCR4, RFTN1, SAMD4A, SAMSN1, SERPINF1, VCAM1, WIPF1 and ZYX (for example upregulation of all of the genes in that group) and downregulation of at least 75% of genes selected from the group consisting of ABCC4, ACAT2, ATP8A1, CANT1, CDH1, DCXR, DHCR24, DHRS7, FAM174B, FAM189A2, FKBP4, FOXA1, GOLM1, GTF3C1, HPN, KIF5C, KLK3, MAP7, MBOAT2, MIOS, MLPH, MY05C, NEDD4L, PART1, PDIA5, PIGH, PMEPA1, PRSS8, SEC23B, SLC43A1, SPDEF, SPINT2, STEAP4, TMPRSS2, TRPM8, TSPAN1, XBP1 (for example upregulation of all of the genes in that group) may be associated with the S8 cancer population. Such a cancer population may be associated with a good prognosis. However, analysis in combination with other markers for prostate cancer (such as Gleason score, PSA etc.) may be done for further confirmation.

Up or downregulation may be in reference to a healthy or control sample. In some embodiments, up or downregulation is with reference to the other cancer classifications.

In one embodiment of the invention, there is provided the use of one of biomarker panels A to F in the diagnosis or classification of prostate cancer. There are also provided methods for diagnosing or classifying prostate cancer by determining the expression status of the genes in one or more of biomarker panels A to F in a patient sample.

References to the use of one of biomarker panels A to F as used in herein, or methods of using such biomarker panels, may refer to the use of at least 75% of the genes in a given biomarker panel. In some embodiments, all of the genes in a given biomarker panel may be used.

Accordingly, in one embodiment there is provided the use of at least 75% of the genes of biomarker panel A (preferably all of the genes of biomarker panel A) in the diagnosis or classification of prostate cancer. There is also provided the use of at least 75% of the genes of biomarker panel B (preferably all of the genes of biomarker panel B) in the diagnosis or classification of prostate cancer. There is also provided the use of at least 75% of the genes of biomarker panel C (preferably all of the genes of biomarker panel C) in the diagnosis or classification of prostate cancer. There is also provided the use of at least 75% of the genes of biomarker panel D (preferably all of the genes of biomarker panel D) in the diagnosis or classification of prostate cancer. There is also provided he use of at least 75% of the genes of biomarker panel E (preferably all of the genes of biomarker panel E) in the diagnosis or classification of prostate cancer. There is also provided he use of at least 75% of the genes of biomarker panel F (preferably all of the genes of biomarker panel F) in the diagnosis or classification of prostate cancer. Such uses may comprises determining the expression status of at least 75% of the genes (for example all of the genes) of a given biomarker panel.

The present invention hence provides the use of any of the biomarker panels in classifying prostate cancer or for diagnosing prostate cancer. The classification or diagnosis is carried out on a patient sample. For example, the expression status (for example level of expression) of the genes from a biomarker panel in a patient sample may be determined. Correlation of the gene expression in the patient sample with the up or downregulation of genes in a biomarker panel as described above may be indicative of that class of prostate cancer. If the class of prostate cancer is associated with a particular prognosis, then the use of the biomarker panel allows a prognosis to be made. The methods may include comparing the level of expression with one or more control genes as discussed herein.

Datasets

The present inventors used MSKCC, CancerMap, Stephenson, CamCap and TOGA as reference datasets in their analysis. However, other suitable datasets are and will become available skilled person. Generally, the datasets comprise a plurality of expression profiles from patient or tumour samples. The size of the dataset can vary. For example, the dataset may comprise expression profiles from at least 20, optionally at least 50, at least 100, at least 200, at least 300, at least 400 or at least 500 patient or tumour samples. Preferably the dataset comprises expression profiles from at least 500 patients or tumours.

In some embodiments, the methods of the invention uses expression profiles from multiple datasets, or reference parameters derived from LPD analysis conducted on multiple datasets. For example, in some embodiments, the methods use expression profiles from at least 2 datasets, each data set comprising expression profiles from at least 250 patients or tumours.

The patient or tumour expression profiles may comprise information on the levels of expression of a subset of genes, for example at least 10, at least 40, at least 100, at least 500, at least 1000, at least 1500, at least 2000, at least 5000 or at least 10000 genes. Preferably, the patient expression profiles comprise expression data for at least 500 genes. In the analysis steps of Methods 2 to 4 of the invention, any selection of a subset of genes will be taken from the genes present in the datasets. Similarly, the provision of the reference variables may be conducted on a subset of genes and/or a subject of expression profiles from the reference dataset.

In methods of the invention, the clinical outcome of the patient samples in the reference dataset may be known. This may be helpful in determining the existence of the different cancer populations in the reference dataset. By “clinical outcome” it is meant that for each patient in the reference dataset whether the cancer has progressed. For example, as part of an initial assessment, those patients may have prostate specific antigen (PSA) levels monitored. When it rises above a specific level, this is indicative of relapse and hence disease progression. Histopathological diagnosis may also be used. Spread to lymph nodes, and metastasis can also be used, as well as death of the patient from the cancer (or simply death of the patient in general) to define the clinical endpoint. Gleason scoring, cancer staging and multiple biopsies (such as those obtained using a coring method involving hollow needles to obtain samples) can be used. Clinical outcomes may also be assessed after treatment for prostate cancer. This is what happens to the patient in the long term. Usually the patient will be treated radically (prostatectomy, radiotherapy) to effectively remove or kill the prostate. The presence of a relapse or a subsequent rise in PSA levels (known as PSA failure) is indicative of progressed cancer.

Control Genes

Note that in any methods of the invention, the statistical analysis can be conducted on the level of expression of the genes being analysed, or the statistical analysis can be conducted on a ratio calculated according to the relative level of expression of the genes and of any control genes.

The control genes (also referred to as housekeeping genes) are useful as they are known not to differ in expression status under the relevant conditions (e.g. DESNT cancer). Exemplary housekeeping genes are known to the skilled person, and they include RPLP2, GAPDH, PGK1 Alas1, TBP1, HPRT, K-Alpha 1. and CLTC. In some embodiments, the housekeeping genes are those listed in Table 3 or Table 4. Table 4 is of particular relevance to prostate cancer. Preferred embodiments of the invention use at least 2 housekeeping genes for this step.

For example, with reference to Method 2, the method may comprise the steps of:
a) providing one or more reference datasets where the cancer classification of each patient sample in the datasets is known (for example as determined by LPD analysis);
b) selecting from this dataset a plurality of genes;
c) applying a LASSO logistic regression model analysis on the selected genes to identify a subset of the selected genes that are predictive of each cancer classification;
d) determining or providing the expression status of at least 1 further, different, gene in the patient sample as a control;
e) determining the relative levels of expression of the subset of genes and of the control gene(s);
f) using the relative expression levels to apply a supervised machine learning algorithm on the dataset to obtain a predictor for each cancer classification;
g) providing a patient expression profile comprising the relative levels of expression in a sample obtained from the patient, wherein the relative levels of expression are obtained using the same subset of genes selected in step c) and the same control gene(s) used in step e);
h) optionally normalising the patient expression profile to the reference dataset(s); and
i) applying the predictor to the patient expression profile to classify the cancer or predict cancer progression.

With reference to Method 3, the method may comprise the steps of:
a) providing one or more reference datasets where the cancer classification of each patient sample in the datasets is known (for example as determined by LPD analysis);
b) selecting from this dataset a plurality of genes, wherein the plurality of genes comprises at least 5, at least 10, at least 20, at least 30, at least 40, at least 50, at least 100, or at least 150 genes selected from the group listed in Table 2;
c) determining or providing the expression status of at least 1 further, different, gene in the patient sample as a control;
d) determining the relative levels of expression of the plurality of genes and of the control gene(s);
e) using the relative levels of expression to apply a supervised machine learning algorithm on the dataset to obtain a predictor for each cancer classification;
f) providing the relative levels of expression of the same plurality of genes and control genes in a sample obtained from the patient to provide a patient expression profile;
g) optionally normalising the patient expression profile to the reference dataset; and
h) applying the predictor to the patient expression profile to classify the cancer, or to predict cancer progression.

With reference to Method 4, the method may comprise the steps of:
a) providing a reference dataset wherein the cancer classification of each patient sample in the dataset is known (for example as determined by LPD analysis);
b) selecting from this dataset of a plurality of genes;
c) determining or providing the expression status of at least 1 further, different, gene in the patient sample as a control;
d) determining the relative levels of expression of the plurality of genes and of the control gene(s);
e) using the relative expression levels of those selected genes to apply a supervised machine learning algorithm on the dataset to obtain a predictor for cancer classification;
f) providing a patient expression profile comprising the relative levels of expression in a sample obtained from the patient, wherein the relative levels of expression is obtained using the same plurality of genes selected in step b) and the same control gene(s) used in step d);
g) optionally normalising the patient expression profile to the reference dataset; and
h) applying the predictor to the patient expression profile to classify the cancer, or to predict cancer progression.

In any of the above methods, the control gene or control genes may be selected from the genes listed in Table 3 or Table 4.

Types of Cancer

The methods and biomarkers disclosed herein are useful in classifying cancers according to their likelihood of progression (and hence are useful in the prognosis of cancer). The present invention is particularly focused on prostate cancer, but the methods can be used for other cancers. Cancers that are likely or will progress are referred to by the inventors as DESNT cancers. References to DESNT cancer herein refer to cancers that are predicted to progress. References to DESNT status herein refer to an indicator of whether or not a cancer will progress. Aggressive cancers are cancers that progress. In one embodiment, the present invention is used to identify or classify metastatic (or potentially metastatic) prostate cancer.

References herein are made to “aggressive cancer” include “aggressive prostate cancer”. Aggressive prostate cancer can be defined as a cancer that requires treatment to prevent, halt or reduce disease progression and potential further complications (such as metastases or metastatic progression). Ultimately, aggressive prostate cancer is prostate cancer that, if left untreated, will spread outside the prostate and may kill the patient. The present invention is useful in detecting some aggressive cancers, including aggressive prostate cancers.

Prostate cancer can be classified according to The American Joint Committee on Cancer (AJCC) tumour-nodes-metastasis (TNM) staging system. The T score describes the size of the main (primary) tumour and whether it has grown outside the prostate and into nearby organs. The N score describes the spread to nearby (regional) lymph nodes. The M score indicates whether the cancer has metastasised (spread) to other organs of the body:

T1 tumours are too small to be seen on scans or felt during examination of the prostate—they may have been discovered by needle biopsy, after finding a raised PSA level. T2 tumours are completely inside the prostate gland and are divided into 3 smaller groups:
T2a—The tumour is in only half of one of the lobes of the prostate gland;
T2b—The tumour is in more than half of one of the lobes;
T2c—The tumour is in both lobes but is still inside the prostate gland.

T3 tumours have broken through the capsule (covering) of the prostate gland—they are divided into 2 smaller groups:
T3a—The tumour has broken through the capsule (covering) of the prostate gland;
T3b—The tumour has spread into the seminal vesicles.

T4 tumours have spread into other body organs nearby, such as the rectum (back passage), bladder, muscles or the sides of the pelvic cavity. Stage T3 and T4 tumours are referred to as locally advanced prostate cancer.

Lymph nodes are described as being ‘positive’ if they contain cancer cells. If a lymph node has cancer cells inside it, it is usually bigger than normal. The more cancer cells it contains, the bigger it will be:
NX—The lymph nodes cannot be checked;
N0—There are no cancer cells in lymph nodes close to the prostate;
N1—There are cancer cells present in lymph nodes.

M staging refers to metastases (cancer spread):
M0—No cancer has spread outside the pelvis;
M1—Cancer has spread outside the pelvis;
M1a—There are cancer cells in lymph nodes outside the pelvis;
M1b—There are cancer cells in the bone;
M1c—There are cancer cells in other places.

Prostate cancer can also be scored using the Gleason grading system, which uses a histological analysis to grade the progression of the disease. A grade of 1 to 5 is assigned to the cells under examination, and the two most common grades are added together to provide the overall Gleason score. Grade 1 closely resembles healthy tissue, including closely packed, well-formed glands, whereas grade 5 does not have any (or very few) recognisable glands. Scores of less than 6 have a good prognosis, whereas scores of 6 or more are classified as more aggressive. The Gleason score was refined in 2005 by the International Society of Urological Pathology and references herein refer to these scoring criteria (Epstein J I, Allsbrook W C Jr, Amin M B, Egevad L L; ISUP Grading Committee. The 2005 International Society of Urological Pathology (ISUP) Consensus Conference on Gleason grading of prostatic carcinoma. Am J Surg Pathol 2005; 29(9):1228-42). The Gleason score is detected in a biopsy, i.e. in the part of the tumour that has been sampled. A Gleason 6 prostate may have small foci of aggressive tumour that have not been sampled by the biopsy and therefore the Gleason is a guide. The lower the Gleason score the smaller the proportion of the patients will have aggressive cancer. Gleason score in a patient with prostate cancer can go down to 2, and up to 10. Because of the small proportion of low Gleasons that have aggressive cancer, the average survival is high, and average survival decreases as Gleason increases due to being reduced by those patients with aggressive cancer (i.e. there is a mixture of survival rates at each Gleason score).

Prostate cancers can also be staged according to how advanced they are. This is based on the TMN scoring as well as any other factors, such as the Gleason score and/or the PSA test. The staging can be defined as follows:

Stage I:
T1, N0, M0, Gleason score 6 or less, PSA less than 10
OR
T2a, N0, M0, Gleason score 6 or less, PSA less than 10

Stage IIA:
T1, N0, M0, Gleason score of 7, PSA less than 20
OR
T1, N0, M0, Gleason score of 6 or less, PSA at least 10 but less than 20:
OR
T2a or T2b, N0, M0, Gleason score of 7 or less, PSA less than 20

Stage IIB:
T2c, N0, M0, any Gleason score, any PSA
OR
T1 or T2, N0, M0, any Gleason score, PSA of 20 or more:
OR
T1 or T2, N0, M0, Gleason score of 8 or higher, any PSA

Stage III:
T3, N0, M0, any Gleason score, any PSA

Stage IV:
T4, N0, M0, any Gleason score, any PSA
OR
Any T, N1, M0, any Gleason score, any PSA:
OR
Any T, any N, M1, any Gleason score, any PSA

In the present invention, an aggressive cancer is defined functionally or clinically: namely a cancer that can progress. This can be measured by PSA failure. When a patient has surgery or radiation therapy, the prostate cells are killed or removed. Since PSA is only made by prostate cells the PSA level in the patient's blood reduces to a very low or undetectable amount. If the cancer starts to recur, the PSA level increases and becomes detectable again. This is referred to as “PSA failure”. An alternative measure is the presence of metastases or death as endpoints.

Increase in Gleason and stage as defined above can also be considered as progression. However, a cancer characterisation is independent of Gleason, stage and PSA. It provides additional information about the likelihood of development of aggressive cancer in addition to Gleason, stage and PSA. It is therefore a useful independent predictor of outcome. Nevertheless, the cancer classification can be combined with Gleason, tumour stage and/or PSA. The cancer classification can also be informative about different drug sensitivities of insensitivities of a patient's cancer according to the prevalence of the different cancer signatures in the patient sample.

Apparatus and Media

In embodiments of the invention, the analysis steps in any of the methods can be computer implemented. For example, the classification step may be computer implemented. The invention also provides a computer readable medium programmed to carry out any of the methods of the invention.

The present invention also provides an apparatus configured to perform any method of the invention.

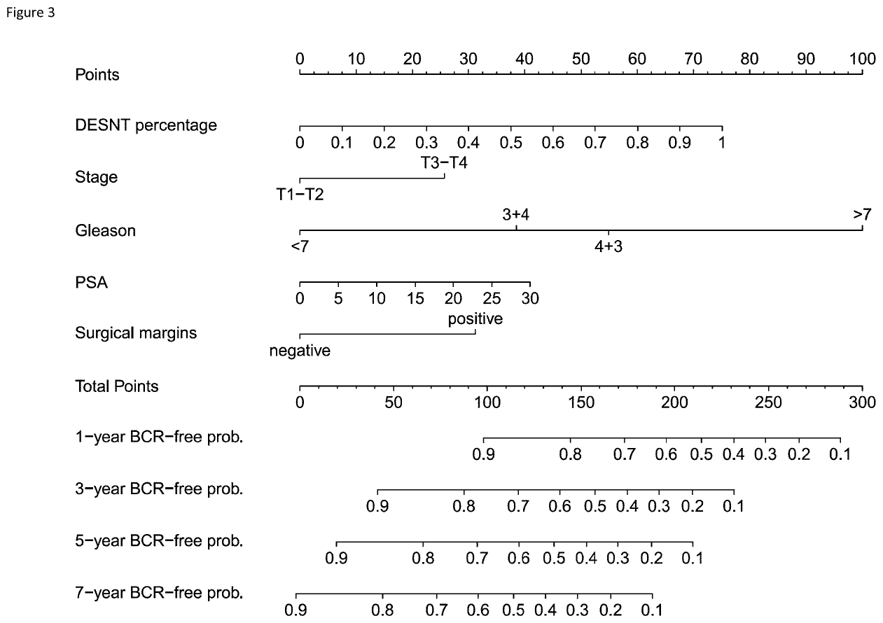

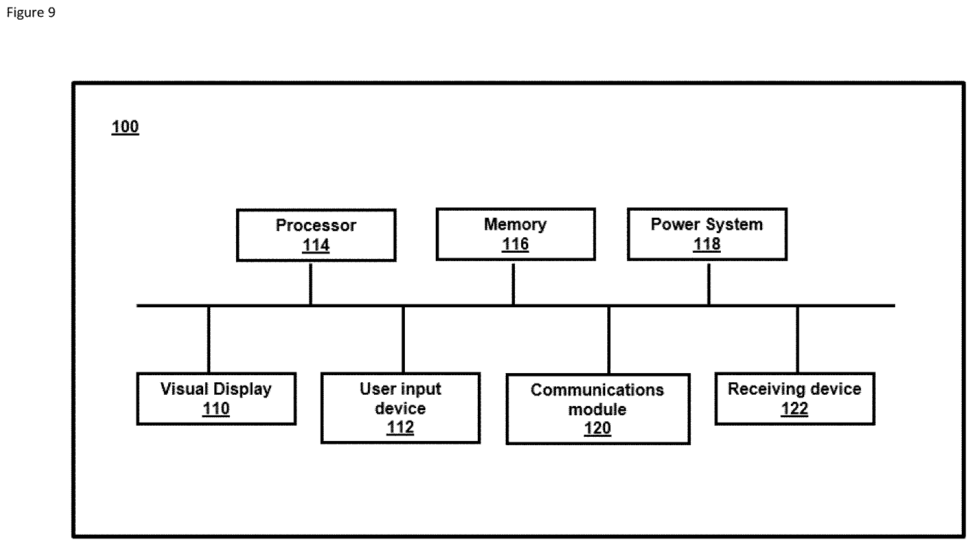

FIG. 9 shows an apparatus or computing device 100 for carrying out a method as disclosed herein. Other architectures to that shown in FIG. 3 may be used as will be appreciated by the skilled person.

Referring to the Figure, the meter 100 includes a number of user interfaces including a visual display 110 and a virtual or dedicated user input device 112. The meter 100 further includes a processor 114, a memory 116 and a power system 118. The meter 100 further comprises a communications module 120 for sending and receiving communications between processor 114 and remote systems. The meter 100 further comprises a receiving device or port 122 for receiving, for example, a memory disk or non-transitory computer readable medium carrying instructions which, when operated, will lead the processor 114 to perform a method as described herein.

The processor 114 is configured to receive data, access the memory 116, and to act upon instructions received either from said memory 116, from communications module 120 or from user input device 112. The processor controls the display 110 and may communicate date to remote parties via communications module 120.

The memory 116 may comprise computer-readable instructions which, when read by the processor, are configured to cause the processor to perform a method as described herein.

The present invention further provides a machine-readable medium (which may be transitory or non-transitory) having instructions stored thereon, the instructions being configured such that when read by a machine, the instructions cause a method as disclosed herein to be carried out.

In one embodiment, there is provided a method of classifying cancer or predicting cancer progression in a patient, the method being implemented by or using at least one processor associated with a memory, the method comprising:
a) providing a set of reference parameters as a first input to the at least one processor, wherein the reference parameters are obtained from a Latent Process Decomposition (LPD) analysis performed on a reference dataset, the reference dataset comprising A expression profiles, each expression profile comprising the expression status of G genes, wherein the reference dataset is decomposed using the LPD analysis into K different cancer expression signatures;
b) obtaining at or providing as a second input to the processor, the expression status of G genes in a sample obtained from the patient to provide a patient expression profile, wherein the G genes in the patient expression profile are the same genes of the reference dataset used to provide the set of reference parameters; and
c) classifying the cancer or predicting cancer progression by the at least one processor, the classification further including:
   a. determining the contribution of each of the K different cancer expression signatures to the patient expression profile using the set of reference parameters provided in step (a).

### Other Methods and Uses of the Invention

The methods of the invention may be combined with a further test to further assist the diagnosis, for example a PSA test, a Gleason score analysis, or a determination of the staging of the cancer. In PSA methods, the amount of prostate specific antigen in a blood sample is quantified. Prostate-specific antigen is a protein produced by cells of the prostate gland. If levels are elevated in the blood, this may be indicative of prostate cancer. An amount that constitutes “elevated” will depend on the specifics of the patient (for example age), although generally the higher the level, the more like it is that prostate cancer is present. A continuous rise in PSA levels over a period of time (for example a week, a month, 6 months or a year) may also be a sign of prostate cancer. A PSA level of more than 4 ng/ml or 10 ng/ml, for example, may be indicative of prostate cancer, although prostate cancer has been found in patients with PSA levels of 4 or less.

In some embodiments of the invention, the methods are able to differentially diagnose aggressive cancer (such as aggressive prostate cancer) from non-aggressive cancer. This can be achieved by determining the classification of the cancer. Alternatively, or additionally, this may be achieved by comparing the level of expression found in the test sample for each of the genes being quantified with that seen in patients presenting with a suitable reference, for example samples from healthy patients, patients suffering from non-aggressive cancer, or using the control or housekeeping genes as discussed herein. In this way, unnecessary treatment can be avoided, and appropriate treatment can be administered instead (for example antibiotic treatment for prostatitis, such as fluoxetine, gabapentin or amitriptyline, or treatment with an alpha reductase inhibitor, such as Finasteride).

In one embodiment of the invention, the method comprises the steps of:
1) detecting RNA in a biological sample obtained from a patient; and
2) quantifying the expression levels of each of the RNA molecules.

The RNA transcripts detected correspond to the biomarkers being quantified (and hence the genes whose expression levels are being measured). In some embodiments, the RNA being detected is the RNA (e.g. mRNA, IncRNA or small RNA) corresponding to at least 5, at least 10, at least 20, at least 30, at least 40, at least 50, at least 100, or at least 150 genes listed in Table 2 (optionally at least all of the genes listed in Table 2). Such methods may be undertaken on a sample previously obtained from a patient, optionally a patient that has undergone a DRE to massage the prostate and increase the amount of RNA in the resulting sample. Alternatively, the method itself may include a step of obtaining a biological sample from a patient.

In one embodiment, the RNA transcripts detected correspond to a selection or all of the genes listed in Table 1. A subset of genes can then be selected for further analysis, such as LPD analysis.

In some embodiments of the invention, the biological sample may be enriched for RNA (or other analyte, such as protein) prior to detection and quantification. The step of enrichment is optional, however, and instead the RNA can be obtained from raw, unprocessed biological samples, such as whole urine. The step of enrichment can be any suitable pre-processing method step to increase the concentration of RNA (or other analyte) in the sample. For example, the step of enrichment may comprise centrifugation and filtration to remove cells from the sample.

In one embodiment of the invention, the method comprises:
a) enriching a biological sample for RNA by amplification, filtration or centrifugation, optionally wherein the biological sample has been obtained from a patient that has undergone DRE;
b) detecting RNA transcripts in the enriched sample; and
c) quantifying the expression levels of each of the detected RNA molecules.

The step of detection may comprise a detection method based on hybridisation, amplification or sequencing, or molecular mass and/or charge detection, or cellular phenotypic change, or the detection of binding of a specific molecule, or a combination thereof. Methods based on hybridisation include Northern blot, microarray, NanoString, RNA-FISH, branched chain hybridisation assay analysis, and related methods. Methods based on amplification include quantitative reverse transcription polymerase chain reaction (qRT-PCT) and transcription mediated amplification, and related methods. Methods based on sequencing include Sanger sequencing, next generation sequencing (high throughput sequencing by synthesis) and targeted RNAseq, nanopore mediated sequencing (MinION), Mass Spectrometry detection and related methods of analysis. Methods based on detection of molecular mass and/or charge of the molecule include, but is not limited to, Mass Spectrometry. Methods based on phenotypic change may detect changes in test cells or in animals as per methods used for screening miRNAs (for example, see Cullen & Arndt, Immunol. Cell Biol., 2005, 83:217-23). Methods based on binding of specific molecules include detection of binding to, for example, antibodies or other binding molecules such as RNA or DNA binding proteins.

In some embodiments, the method may comprise a step of converting RNA transcripts into cDNA transcripts. Such a method step may occur at any suitable time in the method, for example before enrichment (if this step is taking place, in which case the enrichment step is a cDNA enrichment step), before detection (in which case the detection step is a step of cDNA detection), or before quantification (in which case the expression levels of each of the detected RNA molecules by counting the number of transcripts for each cDNA sequence detected).

Methods of the invention may include a step of amplification to increase the amount of RNA or cDNA that is detected and quantified. Methods of amplification include PCR amplification.

In some methods of the invention, detection and quantification of cDNA-binding molecule complexes may be used to determine gene expression. For example, RNA transcripts in a sample may be converted to cDNA by reverse-transcription, after which the sample is contacted with binding molecules specific for the genes being quantified, detecting the presence of a of cDNA-specific binding molecule complex, and quantifying the expression of the corresponding gene.

There is therefore provided the use of cDNA transcripts corresponding to one or more genes identified in the biomarker panels, for use in methods of detecting, diagnosing or determining the prognosis of prostate cancer, in particular prostate cancer.

Once the expression levels are quantified, a diagnosis of cancer (in particular aggressive prostate cancer) can be determined. The methods of the invention can also be used to determine a patient's prognosis, determine a patient's response to treatment or to determine a patient's suitability for treatment for cancer, since the methods can be used to predict cancer progression.

The methods may further comprise the step of comparing the quantified expression levels with a reference and subsequently determining the presence or absence of cancer, in particular aggressive prostate cancer.

Analyte enrichment may be achieved by any suitable method, although centrifugation and/or filtration to remove cell debris from the sample may be preferred. The step of obtaining the RNA from the enriched sample may include harvesting the RNA from microvesicles present in the enriched sample.

The step of sequencing the RNA can be achieved by any suitable method, although direct RNA sequencing, RT-PCR or sequencing-by-synthesis (next generation, or NGS, high-throughput sequencing) may be preferred. Quantification can be achieved by any suitable method, for example counting the number of transcripts identified with a particular sequence. In one embodiment, all the sequences (usually 75-100 base pairs) are aligned to a human reference. Then for each gene defined in an appropriate database (for example the Ensembl database) the number of sequences or reads that overlap with that gene (and don't overlap any other) are counted. To compare a gene between samples it will usually be necessary to normalise each sample so that the amount is the equivalent total amount of sequenced data. Methods of normalisation will be apparent to the skilled person.

As would be apparent to a person of skill in the art, any measurements of analyte concentration may need to be normalised to take in account the type of test sample being used and/or and processing of the test sample that has occurred prior to analysis.

The level of expression of a gene can be compared to a control to determine whether the level of expression is higher or lower in the sample being analysed. If the level of expression is higher in the sample being analysed relative to the level of expression in the sample to which the analysed sample is being compared, the gene is said to be up-regulated. If the level of expression is lower in the sample being analysed relative to the level of expression in the sample to which the analysed sample is being compared, the gene is said to be down-regulated.

In embodiments of the invention, the levels of expression of genes can be prognostic. As such, the present invention is particularly useful in distinguishing prostate cancers requiring intervention (aggressive prostate cancer), and those not requiring intervention (indolent or non-aggressive prostate cancer), avoiding the need for unnecessary procedures and their associated side effects. Drug sensitivities can also be determined using the present invention using known information regarding the sensitivity of certain genes to different drug therapies (i.e. those representative drugable targets) given the contribution of a particular drug sensitive or insensitive group to a patient's cancer.

For example, HDAC1 upregulation is implicated in S3 cancer. Patients whose cancer is classified inot this group may therefore be sensitive to treatment using HDAC1 inhibitors. Many such HDAC1 inhibitors are known, for example, panobinostat. S3 prostate cancers may therefore be sensitive to panobinstat. Moreover, the degree of sensitivity to a given drug treatment may depend on the contribution of the relevant cancer expression signature to the patient's cancer. Therefore, the ability of the present method of the invention to determine the contribution of each cancer expression signature to the patient's cancer is useful in predicting a patient's suitability for and response to particular drug treatments. Accordingly, in some embodiments, the invention provides a method treatment prostate cancer comprising classifying the patient's cancer according to a method of the invention, identifying a drug target associated with the cancer expression signature contributing the most to a patient's cancer expression profile, and administering said drug treatment to the patient.

In some embodiments of the invention, the biomarker panels may be combined with another test such as the PSA test, PCA3 test, Prolaris, or Oncotype DX test. Other tests may be a histological examination to determine the Gleason score, or an assessment of the stage of progression of the cancer.

In a still further embodiment of the invention there is provided a method for determining the suitability of a patient for treatment for prostate cancer, comprising classifying the cancer according to a method of the invention, and deciding whether or not to proceed with treatment for prostate cancer if cancer progression is diagnosed or suspected, in particular if aggressive prostate cancer is diagnosed or suspected.

There is also provided a method of monitoring a patient's response to therapy, comprising classifying the cancer according to a method of the invention using a biological sample obtained from a patient that has previously received therapy for prostate cancer (for example chemotherapy and/or radiotherapy). In some embodiments, the method is repeated in patients before and after receiving treatment. A decision can then be made on whether to continue the therapy or to try an alternative therapy based on the comparison of the levels of expression. For example, if a poor prognosis cancer is detected or suspected (for example a DESNT cancer) after receiving treatment, alternative treatment therapies may be used. Designation as DESNT or as other categories (S1, S2, S3. S4, S5, S6 and S8) may suggest particular therapies. The method can be repeated to see if the treatment is successful at downgrading a patient's cancer from a poor prognosis class to a different class (for example DESNT to non-DESNT).

In one embodiment, there is therefore provided a method comprising:
a) conducting a diagnostic method of the invention of a sample obtained from a patient to determine the class of the cancer;
b) providing treatment for cancer where a poor prognosis class of cancer is found or suspected;
c) subsequently conducting a diagnostic method of the invention of a further sample obtained from a patient to determine the presence or absence of the poor prognosis class of cancer; and
d) maintaining, changing or withdrawing the therapy for cancer.

In some embodiments of the invention, the methods and biomarker panels of the invention are useful for individualising patient treatment, since the effect of different treatments can be easily monitored, for example by measuring biomarker expression in successive urine samples following treatment. The methods and biomarkers of the invention can also be used to predict the effectiveness of treatments, such as responses to hormone ablation therapy.

In another embodiment of the invention there is provided a method of treating or preventing cancer in a patient (such as aggressive prostate cancer), comprising conducting a diagnostic method of the invention of a sample obtained from a patient to classify the cancer, and, if a poor prognosis class of cancer is detected or suspected (for example S7 or S4), administering cancer treatment. Methods of treating prostate cancer may include resecting the tumour and/or administering chemotherapy and/or radiotherapy to the patient.

If possible, treatment for prostate cancer involves resecting the tumour or other surgical techniques. For example, treatment may comprise a radical or partial prostatectomy, trans-urethral resection, orchiectomy or bilateral orchiectomy. Treatment may alternatively or additionally involve treatment by chemotherapy and/or radiotherapy. Chemotherapeutic treatments include docetaxel, abiraterone or enzalutamide. Radiotherapeutic treatments include external beam radiotherapy, pelvic radiotherapy, post-operative radiotherapy, brachytherapy, or, as the case may be, prophylactic radiotherapy. Other treatments include adjuvant hormone therapy (such as androgen deprivation therapy, cryotherapy, high-intensity focused ultrasound, immunotherapy, brachytherapy and/or administration of bisphosphonates and/or steroids.

In another embodiment of the invention, there is provided a method identifying a drug useful for the treatment of cancer, comprising:
a) conducting a diagnostic method of the invention of a sample obtained from a patient to determine the class of the cancer;
b) administering a candidate drug to the patient;
c) subsequently conducting a diagnostic method of the invention on a further sample obtained from a patient to determine the presence or absence of a poor prognosis class of cancer (such as S4 or S7 cancer); and
d) comparing the finding in step (a) with the finding in step (c), wherein a reduction in the prevalence or likelihood of a poor prognosis cancer identifies the drug candidate as a possible treatment for cancer.

The present invention also provides a method of generating report, comprising performing a of classifying prostate cancer or predicting prostate cancer progression in a patient, and providing the results of the classification or prediction in a report. Therefore, in some embodiments, the methods maty further comprise preparing a report providing the results of the classification or cancer progression prediction. The report can be provided to a patient or a patient's physician. The report provides an indication of the cancer classification or severity, or an indication of the probably of cancer progression. Treatment decisions can then be made by the physician for the patient according to the contents of the report. The report may be transmitted electronically (for example by email) or physically (for example by post). The report may comprise one or more treatment recommendations for the patient depending on the classification of the cancer or probability of cancer progression given in the report.

Methods of the present invention may comprise providing a treatment for a cancer patient or suspected cancer patient based on the contents of one or more reports. Alternatively, methods of the present invention may comprise recommending a cancer patient or suspected cancer patient for a particular treatment based on the contents of one or more reports. Methods of the invention may or may not comprise the actual mathematical analysis steps, for example methods of the invention may comprise providing a treatment for a cancer patient or suspected cancer patient or recommending a cancer patient or suspected cancer patient for a particular treatment based on the results of an analysis according to a method of the invention that has been conducted previously. Methods of the invention therefore also comprise providing a treatment for a cancer patient or suspected cancer patient or recommending a cancer patient or suspected cancer patient for a particular treatment, wherein a sample from said patient has been analysed according to a method of the present invention.

Biological Samples

Methods of the invention may comprise steps carried out on biological samples. The biological sample that is analysed may be a urine sample, a semen sample, a prostatic exudate sample, or any sample containing macromolecules or cells originating in the prostate, a whole blood sample, a serum sample, saliva, or a biopsy (such as a prostate tissue sample or a tumour sample). Most commonly for prostate cancer the biological sample is a tissue sample, for example from a prostate biopsy, prostatectomy or TURP. Tissue samples may be preferred. The method may include a step of obtaining or providing the biological sample, or alternatively the sample may have already been obtained from a patient, for example in ex vivo methods. The samples are considered to be representative of the level of expression of the relevant genes in the potentially cancerous prostate tissue, or other cells within the prostate, or microvesicles produced by cells within the prostate or blood or immune system. Hence the methods of the present invention may use quantitative data on RNA produced by cells within the prostate and/or the blood system and/or bone marrow in response to cancer, to determine the presence or absence of prostate cancer.

The methods of the invention may be carried out on one test sample from a patient. Alternatively, a plurality of test samples may be taken from a patient, for example at least 2, 3, 4 or 5 samples. Each sample may be subjected to a separate analysis using a method of the invention, or alternatively multiple samples from a single patient undergoing diagnosis could be included in the method.

The methods of the invention may be conducted in vitro or ex vivo, given they can be done on a sample obtained from a patient. The methods may be considered in vivo if they include a step of obtaining a sample from a patient and/or a step of administering a treatment to a patient.

In some embodiments of the invention, the method is carried out on a tissue sample from a patient, or on the expression status of G genes in a tissue sample obtained from the patient. The expression status of the G genes may be obtained prior to conducting the method of the invention, and then the expression status information is used in the method of the invention.

Further Analytical Methods Used in the Invention

The level of expression of a gene or protein from a biomarker panel of the invention can be determined in a number of ways. Levels of expression may be determined by, for example, quantifying the biomarkers by determining the concentration of protein in the sample, if the biomarkers are expressed as a protein in that sample. Alternatively, the amount of RNA or protein in the sample (such as a tissue sample) may be determined. Once the level of expression has been determined, the level can optionally be compared to a control. This may be a previously measured level of expression (either in a sample from the same subject but obtained at a different point in time, or in a sample from a different subject, for example a healthy subject or a subject with non-aggressive cancer, i.e. a control or reference sample) or to a different protein or peptide or other marker or means of assessment within the same sample to determine whether the level of expression or protein concentration is higher or lower in the sample being analysed. Housekeeping genes can also be used as a control. Ideally, controls are a protein or DNA marker that generally does not vary significantly between samples.

Other methods of quantifying gene expression include RNA sequencing, which in one aspect is also known as whole transcriptome shotgun sequencing (VVTSS). Using RNA sequencing it is possible to determine the nature of the RNA sequences present in a sample, and furthermore to quantify gene expression by measuring the abundance of each RNA molecule (for example, mRNA or microRNA transcripts). The methods use sequencing-by-synthesis approaches to enable high throughout analysis of samples.

There are several types of RNA sequencing that can be used, including RNA PolyA tail sequencing (there the polyA tail of the RNA sequences are targeting using polyT oligonucleotides), random-primed sequencing (using a random oligonucleotide primer), targeted sequence (using specific oligonucleotide primers complementary to specific gene transcripts), small RNA/non-coding RNA sequencing (which may involve isolating small non-coding RNAs, such as microRNAs, using size separation), direct RNA sequencing, and real-time PCR. In some embodiments, RNA sequence reads can be aligned to a reference genome and the number of reads for each sequence quantified to determine gene expression. In some embodiments of the invention, the methods comprise transcription assembly (de-novo or genome-guided).

RNA, DNA and protein arrays (microarrays) may be used in certain embodiments. RNA and DNA microarrays comprise a series of microscopic spots of DNA or RNA oligonucleotides, each with a unique sequence of nucleotides that are able to bind complementary nucleic acid molecules. In this way the oligonucleotides are used as probes to which the correct target sequence will hybridise under high-stringency condition. In the present invention, the target sequence can be the transcribed RNA sequence or unique section thereof, corresponding to the gene whose expression is being detected. Protein microarrays can also be used to directly detect protein expression. These are similar to DNA and RNA microarrays in that they comprise capture molecules fixed to a solid surface.

Capture molecules include antibodies, proteins, aptamers, nucleic acids, receptors and enzymes, which might be preferable if commercial antibodies are not available for the analyte being detected. Capture molecules for use on the arrays can be externally synthesised, purified and attached to the array. Alternatively, they can be synthesised in-situ and be directly attached to the array. The capture molecules can be synthesised through biosynthesis, cell-free DNA expression or chemical synthesis. In-situ synthesis is possible with the latter two.

Once captured on a microarray, detection methods can be any of those known in the art. For example, fluorescence detection can be employed. It is safe, sensitive and can have a high resolution. Other detection methods include other optical methods (for example colorimetric analysis, chemiluminescence, label free Surface Plasmon Resonance analysis, microscopy, reflectance etc.), mass spectrometry, electrochemical methods (for example voltametry and amperometry methods) and radio frequency methods (for example multipolar resonance spectroscopy).

Methods for detection of RNA or cDNA can be based on hybridisation, for example, Northern blot, Microarrays, NanoString, RNA-FISH, branched chain hybridisation assay, or amplification detection methods for quantitative reverse transcription polymerase chain reaction (qRT-PCR) such as TaqMan, or SYBR green product detection. Primer extension methods of detection such as: single nucleotide extension, Sanger sequencing. Alternatively, RNA can be sequenced by methods that include Sanger sequencing, Next Generation (high throughput) sequencing, in particular sequencing by synthesis, targeted RNAseq such as the Precise targeted RNAseq assays, or a molecular sensing device such as the Oxford Nanopore MinION device. Combinations of the above techniques may be utilised such as Transcription Mediated Amplification (TMA) as used in the Gen-Probe PCA3 assay which uses molecule capture via magnetic beads, transcription amplification, and hybridisation with a secondary probe for detection by, for example chemiluminescence.

RNA may be converted into cDNA prior to detection. RNA or cDNA may be amplified prior or as part of the detection.

The test may also constitute a functional test whereby presence of RNA or protein or other macromolecule can be detected by phenotypic change or changes within test cells. The phenotypic change or changes may include alterations in motility or invasion.

Commonly, proteins subjected to electrophoresis are also further characterised by mass spectrometry methods. Such mass spectrometry methods can include matrix-assisted laser desorption/ionisation time-of-flight (MALDI-TOF).

MALDI-TOF is an ionisation technique that allows the analysis of biomolecules (such as proteins, peptides and sugars), which tend to be fragile and fragment when ionised by more conventional ionisation methods. Ionisation is triggered by a laser beam (for example, a nitrogen laser) and a matrix is used to protect the biomolecule from being destroyed by direct laser beam exposure and to facilitate vaporisation and ionisation. The sample is mixed with the matrix molecule in solution and small amounts of the mixture are deposited on a surface and allowed to dry. The sample and matrix co-crystallise as the solvent evaporates.

Additional methods of determining protein concentration include mass spectrometry and/or liquid chromatography, such as LC-MS, UPLC, a tandem UPLC-MS/MS system, and ELISA methods. Other methods that may be used in the invention include Agilent bait capture and PCR-based methods (for example PCR amplification may be used to increase the amount of analyte).

Methods of the invention can be carried out using binding molecules or reagents specific for the analytes (RNA molecules or proteins being quantified). Binding molecules and reagents are those molecules that have an affinity for the RNA molecules or proteins being detected such that they can form binding molecule/reagent-analyte complexes that can be detected using any method known in the art. The binding molecule of the invention can be an oligonucleotide, or oligoribonucleotide or locked nucleic acid or other similar molecule, an antibody, an antibody fragment, a protein, an aptamer or molecularly imprinted polymeric structure, or other molecule that can bind to DNA or RNA. Methods of the invention may comprise contacting the biological sample with an appropriate binding molecule or molecules. Said binding molecules may form part of a kit of the invention, in particular they may form part of the biosensors of in the present invention.

Aptamers are oligonucleotides or peptide molecules that bind a specific target molecule. Oligonucleotide aptamers include DNA aptamer and RNA aptamers. Aptamers can be created by an in vitro selection process from pools of random sequence oligonucleotides or peptides. Aptamers can be optionally combined with ribozymes to self-cleave in the presence of their target molecule. Other oligonucleotides may include RNA molecules that are complimentary to the RNA molecules being quantified. For example, polyT oligos can be used to target the polyA tail of RNA molecules.

Aptamers can be made by any process known in the art. For example, a process through which aptamers may be identified is systematic evolution of ligands by exponential enrichment (SELEX). This involves repetitively reducing the complexity of a library of molecules by partitioning on the basis of selective binding to the target molecule, followed by re-amplification. A library of potential aptamers is incubated with the target protein before the unbound members are partitioned from the bound members. The bound members are recovered and amplified (for example, by polymerase chain reaction) in order to produce a library of reduced complexity (an enriched pool). The enriched pool is used to initiate a second cycle of SELEX. The binding of subsequent enriched pools to the target protein is monitored cycle by cycle. An enriched pool is cloned once it is judged that the proportion of binding molecules has risen to an adequate level. The binding molecules are then analysed individually. SELEX is reviewed in Fitzwater & Polisky (1996) Methods Enzymol, 267:275-301.

Antibodies can include both monoclonal and polyclonal antibodies and can be produced by any means known in the art. Techniques for producing monoclonal and polyclonal antibodies which bind to a particular protein are now well developed in the art. They are discussed in standard immunology textbooks, for example in Roitt et al., Immunology, second edition (1989), Churchill Livingstone, London. The antibodies may be human or humanised, or may be from other species. The present invention includes antibody derivatives that are capable of binding to antigens. Thus, the present invention includes antibody fragments and synthetic constructs. Examples of antibody fragments and synthetic constructs are given in Dougall et al. (1994) Trends Biotechnol, 12:372-379. Antibody fragments or derivatives, such as Fab, F(ab′)₂ or Fv may be used, as may single-chain antibodies (scAb) such as described by Huston et al. (993) Int Rev Immunol, 10:195-217, domain antibodies (dAbs), for example a single domain antibody, or antibody-like single domain antigen-binding receptors. In addition, antibody fragments and immunoglobulin-like molecules, peptidomimetics or non-peptide mimetics can be designed to mimic the binding activity of antibodies. Fv fragments can be modified to produce a synthetic construct known as a single chain Fv (scFv) molecule. This includes a peptide linker covalently joining VH and VL regions which contribute to the stability of the molecule.

Other synthetic constructs include CDR peptides. These are synthetic peptides comprising antigen binding determinants. These molecules are usually conformationally restricted organic rings which mimic the structure of a CDR loop and which include antigen-interactive side chains. Synthetic constructs also include chimeric molecules. Synthetic constructs also include molecules comprising a covalently linked moiety which provides the molecule with some desirable property in addition to antigen binding. For example, the moiety may be a label (e.g. a detectable label, such as a fluorescent or radioactive label), a nucleotide, or a pharmaceutically active agent.

In those embodiments of the invention in which the binding molecule is an antibody or antibody fragment, the method of the invention can be performed using any immunological technique known in the art. For example, ELISA, radio immunoassays or similar techniques may be utilised. In general, an appropriate autoantibody is immobilised on a solid surface and the sample to be tested is brought into contact with the autoantibody. If the cancer marker protein recognised by the autoantibody is present in the sample, an antibody-marker complex is formed. The complex can then be directed or quantitatively measured using, for example, a labelled secondary antibody which specifically recognises an epitope of the marker protein. The secondary antibody may be labelled with biochemical markers such as, for example, horseradish peroxidase (HRP) or alkaline phosphatase (AP), and detection of the complex can be achieved by the addition of a substrate for the enzyme which generates a colorimetric, chemiluminescent or fluorescent product. Alternatively, the presence of the complex may be determined by addition of a marker protein labelled with a detectable label, for example an appropriate enzyme. In this case, the amount of enzymatic activity measured is inversely proportional to the quantity of complex formed and a negative control is needed as a reference to determining the presence of antigen in the sample. Another method for detecting the complex may utilise antibodies or antigens that have been labelled with radioisotopes followed by a measure of radioactivity. Examples of radioactive labels for antigens include ³H, ¹⁴C and ¹²⁵I.

The method of the invention can be performed in a qualitative format, which determines the presence or absence of a cancer marker analyte in the sample, or in a quantitative format, which, in addition, provides a measurement of the quantity of cancer marker analyte present in the sample. Generally, the methods of the invention are quantitative. The quantity of biomarker present in the sample may be calculated using any of the above described techniques. In this case, prior to performing the assay, it may be necessary to draw a standard curve by measuring the signal obtained using the same detection reaction that will be used for the assay from a series of standard samples containing known amounts or concentrations of the cancer marker analyte. The quantity of cancer marker present in a sample to be screened can then extrapolated from the standard curve.

Methods for determining gene expression as used in the present invention therefore include methods based on hybridization analysis of polynucleotides, methods based on sequencing of polynucleotides, proteomics-based methods, reverse transcription PCR, microarray-based methods and immunohistochemistry-based methods. References relating to measuring gene expression are also provided above.

Kit of Parts and Biosensors

In a still further embodiment of the invention there is provided a kit of parts for classifying prostate cancer or predicting prostate cancer progression (for example detecting a class of cancer that is predicted to progress, such as DESNT cancer) comprising a means for quantifying the expression or concentration of the biomarkers of the invention, or means of determining the expression status of the biomarkers of the invention. The means may be any suitable detection means. For example, the means may be a biosensor, as discussed herein. The kit may also comprise a container for the sample or samples and/or a solvent for extracting the biomarkers from the biological sample. The kit may also comprise instructions for use.

In some embodiments of the invention, there is provided a kit of parts for classifying prostate cancer (for example, determining the likelihood of prostate cancer progression) comprising a means for detecting the expression status (for example level of expression) of the biomarkers of the invention. The means for detecting the biomarkers may be reagents that specifically bind to or react with the biomarkers being quantified. Thus, in one embodiment of the invention, there is provided a method of diagnosing prostate cancer comprising contacting a biological sample from a patient with reagents or binding molecules specific for the biomarker analytes being quantified, and measuring the abundance of analyte-reagent or analyte-binding molecule complexes, and correlating the abundance of analyte-reagent or analyte-binding molecule complexes with the level of expression of the relevant protein or gene in the biological sample.

For example, in one embodiment of the invention, the method comprises the steps of:
1. contacting a biological sample with reagents or binding molecules specific for one or more of the biomarkers of the invention;
2. quantifying the abundance of analyte-reagent or analyte-binding molecule complexes for the biomarkers; and
3. correlating the abundance of analyte-reagent or analyte-binding molecule complexes with the expression level of the biomarkers in the biological sample.

The method may further comprise the step of d) comparing the expression level of the biomarkers in step c) with a reference to classify the status of the cancer, in particular to determine the likelihood of cancer progression and hence the requirement for treatment (aggressive prostate cancer). Of course, in some embodiments, the method may additionally comprise conducting a statistical analysis, such as those described in the present invention. The patient can then be treated accordingly. Suitable reagents or binding molecules may include an antibody or antibody fragment, an oligonucleotide, an aptamer, an enzyme, a nucleic acid, an organelle, a cell, a biological tissue, imprinted molecule or a small molecule. Such methods may be carried out using kits of the invention.

The kit of parts may comprise a device or apparatus having a memory and a processor. The memory may have instructions stored thereon which, when read by the processor, cause the processor to perform one or more of the methods described above. The memory may further comprise a plurality of decision trees for use in the random forest analysis.

The kit of parts of the invention may be a biosensor. A biosensor incorporates a biological sensing element and provides information on a biological sample, for example the presence (or absence) or concentration of an analyte. Specifically, they combine a biorecognition component (a bioreceptor) with a physiochemical detector for detection and/or quantification of an analyte (such as RNA or a protein).

The bioreceptor specifically interacts with or binds to the analyte of interest and may be, for example, an antibody or antibody fragment, an enzyme, a nucleic acid (such as an aptamer), an organelle, a cell, a biological tissue, imprinted molecule or a small molecule. The bioreceptor may be immobilised on a support, for example a metal, glass or polymer support, or a 3-dimensional lattice support, such as a hydrogel support.

Biosensors are often classified according to the type of biotransducer present. For example, the biosensor may be an electrochemical (such as a potentiometric), electronic, piezoelectric, gravimetric, pyroelectric biosensor or ion channel switch biosensor. The transducer translates the interaction between the analyte of interest and the bioreceptor into a quantifiable signal such that the amount of analyte present can be determined accurately. Optical biosensors may rely on the surface plasmon resonance resulting from the interaction between the bioreceptor and the analyte of interest. The SPR can hence be used to quantify the amount of analyte in a test sample. Other types of biosensor include evanescent wave biosensors, nanobiosensors and biological biosensors (for example enzymatic, nucleic acid (such as RNA or an aptamer), antibody, epigenetic, organelle, cell, tissue or microbial biosensors).

The invention also provides microarrays (RNA, DNA or protein) comprising capture molecules (such as RNA or DNA oligonucleotides) specific for each of the biomarkers being quantified, wherein the capture molecules are immobilised on a solid support. The microarrays are useful in the methods of the invention.

In one embodiment of the invention, there is provided a method of classifying prostate cancer comprising determining the expression level of one or more of the biomarkers of the invention, and optionally comparing the so determined values to a reference.

The biomarkers that are analysed can be determined according to the Methods of the invention. Alternatively, the biomarker panels provided herein can be used. At least 5, at least 10, at least 20, at least 30, at least 40, at least 50, at least 100, or at least 150 genes of the genes listed in Table 2 (preferably all of them), as well as the biomarkers in biomarker panels A to F, are useful in classifying prostate cancer.

Features for the second and subsequent aspects of the invention are as for the first aspect of the invention mutatis mutandis.

Tables

TABLE 1
500 GENE PROBES THAT VARY IN EXPRESSION
MOST ACROSS THE MSKCC DATASET

| HGNC symbol | Accession ID |
|---|---|
| TGM4 | NM_003241 |
| SERPINB11 | NM_080475 |
| CRISP3 | NM_006061 |
| TDRD1 | NM_198795 |
| SLC14A1 | NM_001128588 |
| IGJ | NM_144646 |
| ERG | NM_001136154 |
| GDEP | NR_026555 |
| TMEFF2 | NM_016192 |
| CST1 | NM_001898 |
| LTF | NM_002343 |
| AMACR | NM_014324 |
| SERPINA3 | NM_001085 |
| NEFH | NM_021076 |
| ACSM1 | NM_052956 |
| OR51E1 | NM_152430 |
| MT1G | NM_005950 |
| ANKRD36B | NM_025190 |
| LOC100510059 | XM_003120411 |
| PLA2G2A | NM_000300 |
| TARP | NM_001003799 |
| REXO1L1 | NM_172239 |
| ANPEP | NM_001150 |
| HLA-DRB5 | NM_002125 |
| PLA2G7 | NM_001168357 |
| NCAPD3 | NM_015261 |
| OR51F2 | NM_001004753 |
| SPINK1 | NM_003122 |
| RCN1 | NM_002901 |
| CP | NM_000096 |
| SMU1 | NM_018225 |
| ACTC1 | NM_005159 |
| AGR2 | NM_006408 |
| SLC26A4 | NM_000441 |
| IGKC | BC032451 |
| MYBPC1 | NM_002465 |
| NPY | NM_000905 |
| PI15 | NM_015886 |
| SLC22A3 | NM_021977 |
| PIGR | NM_002644 |
| MME | NM_007288 |
| RBPMS | L17325 |
| HLA-DRB1 | NM_002124 |
| FOLH1 | NM_001193471 |
| LUZP2 | NM_001009909 |
| MSMB | NM_002443 |
| GSTT1 | NM_000853 |
| MMP7 | NM_002423 |
| NR4A2 | NM_006186 |
| ARG2 | NM_001172 |
| ZNF385B | NM_152520 |
| RGS1 | NM_002922 |
| DNAH5 | NM_001369 |
| NPR3 | NM_000908 |
| RAB3B | NM_002867 |
| CHRDL1 | NM_145234 |
| ZNF208 | NM_007153 |
| MBOAT2 | NM_138799 |
| ATF3 | NM_001040619 |
| ST6GAL1 | NM_173216 |
| GDF15 | NM_004864 |
| ANXA1 | NM_000700 |
| FOLH1 | NM_004476 |
| C4B | NM_001002029 |
| ELOVL2 | NM_017770 |
| GSTM1 | NM_000561 |
| GLIPR1 | NM_006851 |
| C3 | NM_000064 |
| MYO6 | NM_004999 |
| ORM2 | NM_000608 |
| RAET1L | NM_130900 |
| PCDHB3 | NM_018937 |
| C1orf150 | ENST00000366488 |
| ALOX15B | NM_001141 |
| LSAMP | NM_002338 |
| SLC15A2 | NM_021082 |
| PCP4 | NM_006198 |
| MCCC2 | NM_022132 |
| GCNT1 | NM_001097634 |
| C5orf23 | BC022250 |
| SCGB1D2 | NM_006551 |
| CXCL2 | NM_002089 |
| AFF3 | NM_001025108 |
| ATP8A2 | NM_016529 |
| PRIM2 | NM_000947 |
| ADAMTSL1 | NM_001040272 |
| NELL2 | NM_001145108 |
| RPS4Y1 | NM_001008 |
| CD24 | NM_013230 |
| GOLGA6L9 | NM_198181 |
| ZFP36 | NM_003407 |
| TRIB1 | NM_025195 |
| BNIP3 | NM_004052 |
| KL | NM_004795 |
| PDE5A | NM_001083 |
| TAS2R4 | NM_016944 |
| SEPP1 | NM_001093726 |
| GREM1 | NM_013372 |
| RASD1 | NM_016084 |
| C1S | NM_201442 |
| CLSTN2 | NM_022131 |
| DMXL1 | NM_005509 |
| HIST1H2BC | NM_003526 |
| NRG4 | NM_138573 |
| ARL17A | NM_001113738 |
| GRPR | NM_005314 |
| PART1 | NR_024617 |
| CYP3A5 | NR_033807 |
| KCNC2 | NM_139136 |
| SERPINE1 | NM_000602 |
| SLC6A14 | NM_007231 |
| EIF4A1 | NM_001416 |
| MYOF | NM_013451 |
| PHOSPHO2 | NM_001008489 |
| GCNT2 | NM_145649 |
| AOX1 | NM_001159 |
| CCDC80 | NM_199511 |
| ATP2B4 | NM_001001396 |
| UGDH | NM_003359 |
| GSTM2 | NM_000848 |
| MEIS2 | NM_172316 |
| RGS2 | NM_002923 |
| PRKG2 | NM_006259 |
| FIBIN | NM_203371 |
| FDXACB1 | NM_138378 |
| SOD2 | NM_001024465 |
| SEPT7 | NM_001788 |
| PTPRC | NM_002838 |
| GABRP | NM_014211 |
| CBWD3 | NM_201453 |
| TOR1AIP2 | NM_022347 |
| CXCR4 | NM_001008540 |
| OR51L1 | NM_001004755 |
| SLC12A2 | NM_001046 |
| AGAP11 | NM_133447 |
| SLC27A2 | NM_003645 |
| AZGP1 | NM_001185 |
| VCAN | NM_004385 |
| ERAP2 | NM_022350 |
| KRT17 | NM_000422 |
| SLC2A12 | NM_145176 |
| CCL4 | NM_002984 |
| RPF2 | NM_032194 |
| S100A10 | NM_002966 |
| PMS2CL | NR_002217 |
| MMP2 | NM_004530 |
| SLC8A1 | NM_021097 |
| OAS2 | NM_002535 |
| ARRDC3 | NM_020801 |
| AMY2B | NM_020978 |
| SPARCL1 | NM_001128310 |
| IQGAP2 | NM_006633 |
| ACAD8 | NM_014384 |
| LPAR3 | NM_012152 |
| HIGD2A | NM_138820 |
| NUCB2 | NM_005013 |
| HLA-DPA1 | NM_033554 |
| SLITRK6 | NM_032229 |
| TPM2 | NM_003289 |
| REPS2 | NM_004726 |
| EAF2 | NM_018456 |
| CAV1 | NM_001172895 |
| PRUNE2 | NM_015225 |
| TMEM178 | NM_152390 |
| MFAP4 | NM_001198695 |
| SYNM | NM_145728 |
| EFEMP1 | NM_004105 |
| RND3 | NM_005168 |
| SCNN1A | NM_001038 |
| B3GNT5 | NM_032047 |
| LMOD1 | NM_012134 |
| UBC | NM_021009 |
| LMO3 | NM_018640 |
| LOX | NM_002317 |
| NFIL3 | NM_005384 |
| C11orf92 | NR_034154 |
| C11orf48 | NM_024099 |
| BCAP29 | NM_018844 |
| EPCAM | NM_002354 |
| PTGDS | NM_000954 |
| ASB5 | NM_080874 |
| TUBA1B | NM_006082 |
| SERHL | NR_027786 |
| ITGA5 | NM_002205 |
| SPARC | NM_003118 |
| LOC286161 | AK091672 |
| NAALADL2 | NM_207015 |
| TMPRSS2 | NM_001135099 |
| SERPINF1 | NM_002615 |
| EPHA7 | NM_004440 |
| SDAD1 | NM_018115 |
| RLN1 | NM_006911 |
| ORM1 | NM_000607 |
| ODZ1 | NM_001163278 |
| ACTB | NM_001101 |
| SPON2 | NM_012445 |
| SLC38A11 | NM_173512 |
| FOS | NM_005252 |
| OR51T1 | NM_001004759 |
| HLA-DMB | NM_002118 |
| KRT15 | NM_002275 |
| ITGA8 | NM_003638 |
| CXADR | NM_001338 |
| LYZ | NM_000239 |
| CEACAM20 | NM_001102597 |
| C8orf4 | NM_020130 |
| DPP4 | NM_001935 |
| PGC | NM_002630 |
| C15orf21 | NR_022014 |
| CHORDC1 | NM_012124 |
| LRRN1 | NM_020873 |
| MT1M | NM_176870 |
| EPHA6 | NM_001080448 |
| PDE11A | NM_001077197 |
| TMSB15A | NM_021992 |
| LYPLA1 | NM_006330 |
| FOSB | NM_006732 |
| F5 | NM_000130 |
| C15orf48 | NM_032413 |
| MIPEP | NM_005932 |
| HSD17B6 | NM_003725 |
| SLPI | NM_003064 |
| CD38 | NM_001775 |
| MMP23B | NM_006983 |
| OR51A7 | NM_001004749 |
| CFB | NM_001710 |
| CCL2 | NM_002982 |
| POTEM | NM_001145442 |
| TPMT | NM_000367 |
| FAM3B | NM_058186 |
| FLRT3 | NM_198391 |
| C7 | NM_000587 |
| NTN4 | NM_021229 |
| FAM36A | NM_198076 |
| CNTNAP2 | NM_014141 |
| SC4MOL | NM_006745 |
| CH17-189H20.1 | AK000992 |
| TRGC2 | ENST00000427089 |
| RAP1B | NM_015646 |
| SLC4A4 | NM_001098484 |
| DCN | NM_001920 |
| LDHB | NM_001174097 |
| PCDHB5 | NM_015669 |
| ACADL | NM_001608 |
| ZNF99 | NM_001080409 |
| CPNE4 | NM_130808 |
| CCDC144B | NR_036647 |
| SLC26A2 | NM_000112 |
| CYP1B1 | NM_000104 |
| SELE | NM_000450 |
| CLDN1 | NM_021101 |
| KRT13 | NM_153490 |
| SFRP2 | NM_003013 |
| SLC25A33 | NM_032315 |
| HSD17B11 | NM_016245 |
| HSD17B13 | NM_178135 |
| UGT2B4 | NM_021139 |
| CTGF | NM_001901 |
| SCIN | NM_001112706 |
| C10orf81 | NM_001193434 |
| CYR61 | NM_001554 |
| PRUNE2 | NM_015225 |
| IFI6 | NM_002038 |
| MYH11 | NM_022844 |
| PPP1R3C | NM_005398 |
| KCNH8 | NM_144633 |
| ZNF615 | NM_198480 |
| ERV3 | NM_001007253 |
| F3 | NM_001993 |
| TTN | NM_133378 |
| LYRM5 | NM_001001660 |
| FMOD | NM_002023 |
| NEXN | NM_144573 |
| IL28A | NM_172138 |
| FHL1 | NM_001159702 |
| CXCL10 | NM_001565 |
| SPOCK1 | NM_004598 |
| GSTP1 | NM_000852 |
| OAT | NM_000274 |
| HIST2H2BF | NM_001024599 |
| ACSM3 | NM_005622 |
| GLB1L3 | NM_001080407 |
| SLC5A1 | NM_000343 |
| OR4N4 | NM_001005241 |
| MAOB | NM_000898 |
| BZW1 | NM_014670 |
| GENSCAN00000007309 | GENSCAN00000007309 |
| SLC45A3 | NM_033102 |
| SEC11C | NM_033280 |
| IFIT1 | NM_001548 |
| PAK1IP1 | NM_017906 |
| HIST1H3C | NM_003531 |
| ERRFI1 | NM_018948 |
| ADAMTS1 | NM_006988 |
| TRIM36 | NM_018700 |
| FLNA | NM_001456 |
| CCND2 | NM_001759 |
| IFIT3 | NM_001031683 |
| FN1 | NM_212482 |
| PRY | NM_004676 |
| HSPB8 | NM_014365 |
| CD177 | NM_020406 |
| TP63 | NM_003722 |
| IFI44 | NM_006417 |
| COL12A1 | NM_004370 |
| EDNRA | NM_001957 |
| PCDHB2 | NM_018936 |
| HLA-DRA | NM_019111 |
| TUBA3E | NM_207312 |
| ASPN | NM_017680 |
| FAM127A | NM_001078171 |
| DMD | NM_000109 |
| DHRS7 | NM_016029 |
| ANO7 | NM_001001891 |
| MEIS1 | NM_002398 |
| TSPAN1 | NM_005727 |
| CNTN1 | NM_001843 |
| TRIM22 | NM_006074 |
| GSTA2 | NM_000846 |
| SORBS1 | NM_001034954 |
| GPR81 | NM_032554 |
| CSRP1 | NM_004078 |
| C3orf14 | AF236158 |
| FGFR2 | NM_000141 |
| SNAI2 | NM_003068 |
| CALCRL | NM_005795 |
| MON1B | NM_014940 |
| PVRL3 | NM_015480 |
| VGLL3 | NM_016206 |
| SULF1 | NM_001128205 |
| LIFR | NM_002310 |
| SH3RF1 | AB062480 |
| C12orf75 | NM_001145199 |
| GNPTAB | NM_024312 |
| CALM2 | NM_001743 |
| SOX14 | NM_004189 |
| RPL35 | NM_007209 |
| HSPA1B | NM_005346 |
| MSN | NM_002444 |
| MTRF1L | NM_019041 |
| PTN | NM_002825 |
| CAMKK2 | NM_006549 |
| RBM7 | NM_016090 |
| OR52H1 | NM_001005289 |
| C1R | NM_001733 |
| CHRNA2 | NM_000742 |
| MRPL41 | NM_032477 |
| PROM1 | NM_001145847 |
| LPAR6 | NM_005767 |
| SAMHD1 | NM_015474 |
| SCNN1G | NM_001039 |
| DNAJC10 | NM_018981 |
| MOXD1 | NM_015529 |
| HIST1H2BG | NM_003518 |
| ID1 | NM_181353 |
| SEMA3C | NM_006379 |
| OLFM4 | NM_006418 |
| OR51E2 | NM_030774 |
| LCE2D | NM_178430 |
| EGR1 | NM_001964 |
| MT1L | NR_001447 |
| SCUBE2 | NM_020974 |
| FAM55D | NM_001077639 |
| PDK4 | NM_002612 |
| CXCL13 | NM_006419 |
| CACNA1D | NM_000720 |
| GPR160 | NM_014373 |
| CPM | NM_001874 |
| PTGS2 | NM_000963 |
| TSPAN8 | NM_004616 |
| BMP5 | NM_021073 |
| GOLGA8A | NR_027409 |
| OR4N2 | NM_001004723 |
| FAM135A | NM_001105531 |
| DYNLL1 | NM_001037494 |
| DSC3 | NM_024423 |
| C4orf3 | NM_001001701 |
| HIST1H2BK | NM_080593 |
| LCN2 | NM_005564 |
| STEAP4 | NM_024636 |
| RPS27L | NM_015920 |
| TRPM8 | NM_024080 |
| ID2 | NM_002166 |
| LUM | NM_002345 |
| EDNRB | NM_001122659 |
| PGM5 | NM_021965 |
| SFRP4 | NM_003014 |
| STEAP1 | NM_012449 |
| FADS2 | NM_004265 |
| CXCL11 | NM_005409 |
| CWH43 | NM_025087 |
| SNRPN | BC043194 |
| GPR110 | NM_153840 |
| THBS1 | NM_003246 |
| APOD | NM_001647 |
| HPGD | NM_000860 |
| LEPREL1 | NM_018192 |
| LCE1D | NM_178352 |
| GSTM5 | NM_000851 |
| SLC30A4 | NM_013309 |
| SEMA3D | NM_152754 |
| CACNA2D1 | NM_000722 |
| GPR116 | NM_015234 |
| C7orf63 | NM_001039706 |
| FAM198B | NM_001128424 |
| SCD | NM_005063 |
| IFI44L | NM_006820 |
| KRT5 | NM_000424 |
| SCN7A | NM_002976 |
| GOLM1 | NM_016548 |
| HIST4H4 | NM_175054 |
| IL7R | NM_002185 |
| CSGALNACT1 | NM_018371 |
| A2M | NM_000014 |
| LRRC9 | AK128037 |
| ARHGEF38 | NM_017700 |
| ACSL5 | NM_016234 |
| SGK1 | NM_001143676 |
| TMEM45B | NM_138788 |
| AHNAK2 | NM_138420 |
| NEDD8 | NM_006156 |
| GREB1 | NM_014668 |
| UBQLN4 | NM_020131 |
| SDHC | NM_003001 |
| TCEAL2 | NM_080390 |
| SLC18A2 | NM_003054 |
| HIST1H2BE | NM_003523 |
| RARRES1 | NM_206963 |
| PLN | NM_002667 |
| OGN | NM_033014 |
| GPR110 | NM_025048 |
| CLGN | NM_001130675 |
| NIPAL3 | NM_020448 |
| ACTG2 | NM_001615 |
| RCAN3 | NM_013441 |
| KLK11 | NM_001167605 |
| HMGCS2 | NM_005518 |
| EML5 | NM_183387 |
| EDIL3 | NM_005711 |
| PIGH | NM_004569 |
| GLYATL1 | NM_080661 |
| ATP1B1 | NM_001677 |
| GJA1 | NM_000165 |
| PLA1A | NM_015900 |
| MPPED2 | NM_001584 |
| AMD1 | NM_001634 |
| EMP1 | NM_001423 |
| PRR16 | NM_016644 |
| CNN1 | NM_001299 |
| GHR | NM_000163 |
| ALDH1A1 | NM_000689 |
| TRIM29 | NM_012101 |
| IFNA17 | NM_021268 |
| KLF6 | NM_001300 |
| C7orf58 | NM_024913 |
| RDH11 | NM_016026 |
| NR4A1 | NM_002135 |
| RWDD4 | NM_152682 |
| ABCC4 | NM_005845 |
| ZNF91 | NM_003430 |
| GABRE | NM_004961 |
| SLC16A1 | NM_001166496 |
| DEGS1 | NM_003676 |
| CLDN8 | NM_199328 |
| HAS2 | NM_005328 |
| ODC1 | NM_002539 |
| REEP3 | NM_001001330 |
| LYRM4 | AF258559 |
| PPFIA2 | NM_003625 |
| PGM3 | NM_015599 |
| ZDHHC8P1 | NR_003950 |
| C6orf72 | AY358952 |
| HIST1H2BD | NM_138720 |
| TES | NM_015641 |
| PDE8B | NM_003719 |
| DNAJB4 | NM_007034 |
| RGS5 | NM_003617 |
| EPHA3 | NM_005233 |
| COX7A2 | NR_029466 |
| MT1H | NM_005951 |
| HIST2H2BE | NM_003528 |
| TGFB3 | NM_003239 |
| VEGFA | NM_001025366 |
| CRISPLD2 | NM_031476 |
| TFF1 | NM_003225 |
| LOC100128816 | AY358109 |
| SYT1 | NM_001135805 |
| CPE | NM_001873 |
| TRPC4 | NM_016179 |
| RAB27A | NM_004580 |
| CD69 | NM_001781 |
| RPL17 | NM_000985 |
| PSCA | NM_005672 |
| ATRNL1 | NM_207303 |
| MYOCD | NM_001146312 |
| MS4A8B | NM_031457 |
| TNS1 | NM_022648 |
| BAMBI | NM_012342 |
| IGF1 | NM_001111283 |
| RALGAPA1 | NM_014990 |

TABLE 2
Genes that are predictive of cancer classification,
as identified by LASSO

| | | | | |
|---|---|---|---|---|
| CELA3A | MUC13 | HLA-B | MMP26 | CFDP1 |
| CD52 | CNBP | CYP39A1 | PRR5L | ZNF286A |
| RBBP4 | A4GNT | PLA2G7 | TRIM48 | RND2 |
| ANGPTL3 | EHHADH | BMP5 | FAM111A | ARL4D |
| DPH5 | GBA3 | DST | CRTAM | ADAM11 |
| GSTM2 | STAP1 | HECW1 | MPPED2 | RGS9 |
| PIP5K1A | COX7A2 | CCT6A | PYGM | CLEC10A |
| CASQ1 | UGT8 | ELN | GUCY1A2 | C17orf59 |
| PPOX | NDST3 | GTF2IRD1 | H2AFJ | MEOX1 |
| SDHC | POU4F2 | ASB4 | SLCO1B3 | GH1 |
| CAPN9 | KLHL2 | FBXO24 | HSD17B6 | DCXR |
| RPE65 | TLL1 | LRRC17 | KLRB1 | NOL4 |
| BCL10 | INTS12 | GPR22 | PRB4 | FFAR2 |
| SLC16A1 | GYPB | BCAP29 | GYS2 | IRGC |
| NUCKS1 | DCHS2 | DNAJB9 | LDHB | ZNF613 |
| DUSP10 | FGG | CALU | FAM60A | SPTLC3 |
| ZNF706 | NPR3 | RNF32 | RND1 | CST7 |
| COLEC11 | GHR | SOSTDC1 | KCNC2 | MYL9 |
| MXD1 | PDE8B | HGF | RPL6 | TGIF2 |
| ACTG2 | SLC22A4 | TFPI2 | PCDH17 | CEBPB |
| IL1RL2 | SPINK5 | CYP3A7 | GPC5 | CST5 |
| IL1RL1 | GABRA6 | RELN | FOXO1 | DGCR6L |
| DBI | SEMA5A | PTN | PCDH9 | GSTT1 |

TABLE 3
Example Control Genes: House Keeping Control genes

HPRT
B2M
TBP
GAPDH
ALAS1
RPLP2
KLK3_ex2-3
KLK3_ex1-2
SDH1
GPI
PSMB2
PSMB4
RAB7A
REEP5
18S rRNA
28s rRNA
PBGD
ACTB
UBC
rb 23 kDa
TUBA1
RPS9
TFR
RPS13
RPL27
RPS20
RPL30
RPL13A
RPL9
SRP14
RPL24
RPL22
RPS29
RPS16
RPL4
RPL6
OAZ1
RPS12
LDHA
PGAM1
PGK1
VIM
PFKP
EF-1d
IMPDH1
IDH2
KGDHC
SRF7
RPLP0
ALDOA
COX
AST
MDH
EIF4A1
FH
ATP5F1
H2A.X
IMP
accession
number
X56932
ODC-AZ
PDHA1
PLA2
PMI1
SRP75
RPL3
RPL32
RPL7a
RNAP II
RPL10
RPL23a
RPL37
RPS11
RPS3
SDHB
SNRPB
SDH
TCP20
CLTC

TABLE 4
Example Control Genes: Prostate specific control transcripts

| | | |
|---|---|---|
| KLK2 | TGM4 | HOXB13 |
| KLK3 | RLN1 | PMEPA1 |
| KLK4 | ACPP | PAP |
| FOLH1(PSMA) | PTI-1 | STEAP1 |
| PCGEM1 | PSCA | SPINK1 |
| PCA3 | NKX3.1 | |
| TMPRSS2 | SPDEF | |
| TMPRSS2/ERG | PMA | |

TABLE 5
Up and downregulation of genes in some of the different prostate cancer populations.

| | | |
|---|---|---|
| Gene | +/− | Description |

Cancer population S2

| | | |
|---|---|---|
| KRT13 | + | keratin 13 [Source: HGNC Symbol; Acc: HGNC: 6415] |
| TGM4 | + | transglutaminase 4 [Source: HGNC Symbol; Acc: HGNC: 11780] |

Cancer population S3

| | | |
|---|---|---|
| CSGALNACT1 | + | chondroitin sulfate N-acetylgalactosaminyltransferase 1 [Source: HGNC Symbol; Acc: HGNC: 24290] |
| ERG | + | ERG, ETS transcription factor [Source: HGNC Symbol; Acc: HGNC: 3446] |
| GHR | + | growth hormone receptor [Source: HGNC Symbol; Acc: HGNC: 4263] |
| GUCY1A3 | + | guanylate cyclase 1 soluble subunit alpha [Source: HGNC Symbol; Acc: HGNC: 4685] |
| HDAC1 | + | histone deacetylase 1 [Source: HGNC Symbol; Acc: HGNC: 4852] |
| ITPR3 | + | inositol 1,4,5-trisphosphate receptor type 3 [Source: HGNC Symbol; Acc: HGNC: 6182] |
| PLA2G7 | + | phospholipase A2 group VII [Source: HGNC Symbol; Acc: HGNC: 9040] |

Cancer population S5

| | | |
|---|---|---|
| ABHD2 | + | abhydrolase domain containing 2 [Source: HGNC Symbol; Acc: HGNC: 18717] |
| ACAD8 | + | acyl-CoA dehydrogenase family member 8 [Source: HGNC Symbol; Acc: HGNC: 87] |
| ACLY | + | ATP citrate lyase [Source: HGNC Symbol; Acc: HGNC: 115] |
| ALCAM | + | activated leukocyte cell adhesion molecule [Source: HGNC Symbol; Acc: HGNC: 400] |
| ALDH6A1 | + | aldehyde dehydrogenase 6 family member A1 [Source: HGNC Symbol; Acc: HGNC: 7179] |
| ALOX15B | + | arachidonate 15-lipoxygenase, type B [Source: HGNC Symbol; Acc: HGNC: 434] |
| ARHGEF7 | + | Rho guanine nucleotide exchange factor 7 [Source: HGNC Symbol; Acc: HGNC: 15607] |
| AUH | + | AU RNA binding methylglutaconyl-CoA hydratase [Source: HGNC Symbol; Acc: HGNC: 890] |
| BBS4 | + | Bardet-Biedl syndrome 4 [Source: HGNC Symbol; Acc: HGNC: 969] |
| C1orf115 | + | chromosome 1 open reading frame 115 [Source: HGNC Symbol; Acc: HGNC: 25873] |
| CAMKK2 | + | calcium/calmodulin dependent protein kinase kinase 2 [Source: HGNC Symbol; Acc: HGNC: 1470] |
| COG5 | + | component of oligomeric golgi complex 5 [Source: HGNC Symbol; Acc: HGNC: 14857] |
| CPEB3 | + | cytoplasmic polyadenylation element binding protein 3 [Source: HGNC Symbol; Acc: HGNC: 21746] |
| CYP2J2 | + | cytochrome P450 family 2 subfamily J member 2 [Source: HGNC Symbol; Acc: HGNC: 2634] |
| DHRS3 | − | dehydrogenase/reductase 3 [Source: HGNC Symbol; Acc: HGNC: 17693] |
| DHX32 | + | DEAH-box helicase 32 (putative) [Source: HGNC Symbol; Acc: HGNC: 16717] |
| EHHADH | + | enoyl-CoA hydratase and 3-hydroxyacyl CoA dehydrogenase [Source: HGNC Symbol; Acc: HGNC: 3247] |
| ELOVL2 | + | ELOVL fatty acid elongase 2 [Source: HGNC Symbol; Acc: HGNC: 14416] |
| ERG | − | ERG, ETS transcription factor [Source: HGNC Symbol; Acc: HGNC: 3446] |
| EXTL2 | + | exostosin like glycosyltransferase 2 [Source: HGNC Symbol; Acc: HGNC: 3516] |
| F3 | − | coagulation factor III, tissue factor [Source: HGNC Symbol; Acc: HGNC: 3541] |
| FAM111A | + | family with sequence similarity 111 member A [Source: HGNC Symbol; Acc: HGNC: 24725] |
| GATA3 | − | GATA binding protein 3 [Source: HGNC Symbol; Acc: HGNC: 4172] |
| GLUD1 | + | glutamate dehydrogenase 1 [Source: HGNC Symbol; Acc: HGNC: 4335] |
| GNMT | + | glycine N-methyltransferase [Source: HGNC Symbol; Acc: HGNC: 4415] |
| HES1 | − | hes family bHLH transcription factor 1 [Source: HGNC Symbol; Acc: HGNC: 5192] |
| HPGD | + | hydroxyprostaglandin dehydrogenase 15-(NAD) [Source: HGNC Symbol; Acc: HGNC: 5154] |
| KHDRBS3 | − | KH RNA binding domain containing, signal transduction associated 3 [Source: HGNC Symbol; Acc: HGNC: 18117] |
| LAMB2 | − | laminin subunit beta 2 [Source: HGNC Symbol; Acc: HGNC: 6487] |
| LAMC2 | − | laminin subunit gamma 2 [Source: HGNC Symbol; Acc: HGNC: 6493] |
| MIPEP | + | mitochondrial intermediate peptidase [Source: HGNC Symbol; Acc: HGNC: 7104] |
| MON1B | + | MON1 homolog B, secretory trafficking associated [Source: HGNC Symbol; Acc: HGNC: 25020] |
| NANS | + | N-acetylneuraminate synthase [Source: HGNC Symbol; Acc: HGNC: 19237] |
| NAT1 | + | N-acetyltransferase 1 [Source: HGNC Symbol; Acc: HGNC: 7645] |
| NCAPD3 | + | non-SMC condensin II complex subunit D3 [Source: HGNC Symbol; Acc: HGNC: 28952] |
| PDE8B | − | phosphodiesterase 8B [Source: HGNC Symbol; Acc: HGNC: 8794] |
| PPFIBP2 | + | PPFIA binding protein 2 [Source: HGNC Symbol; Acc: HGNC: 9250] |
| PTK7 | − | protein tyrosine kinase 7 (inactive) [Source: HGNC Symbol; Acc: HGNC: 9618] |
| PTPN13 | + | protein tyrosine phosphatase, non-receptor type 13 [Source: HGNC Symbol; Acc: HGNC: 9646] |
| PTPRM | + | protein tyrosine phosphatase, receptor type M [Source: HGNC Symbol; Acc: HGNC: 9675] |
| RAB27A | + | RAB27A, member RAS oncogene family [Source: HGNC Symbol; Acc: HGNC: 9766] |
| REPS2 | + | RALBP1 associated Eps domain containing 2 [Source: HGNC Symbol; Acc: HGNC: 9963] |
| RFX3 | + | regulatory factor X3 [Source: HGNC Symbol; Acc: HGNC: 9984] |
| SCIN | + | scinderin [Source: HGNC Symbol; Acc: HGNC: 21695] |
| SLC1A1 | + | solute carrier family 1 member 1 [Source: HGNC Symbol; Acc: HGNC: 10939] |
| SLC4A4 | + | solute carrier family 4 member 4 [Source: HGNC Symbol; Acc: HGNC: 11030] |
| SMPDL3A | + | sphingomyelin phosphodiesterase acid like 3A [Source: HGNC Symbol; Acc: HGNC: 17389] |
| SORL1 | − | sortilin related receptor 1 [Source: HGNC Symbol; Acc: HGNC: 11185] |
| STXBP6 | + | syntaxin binding protein 6 [Source: HGNC Symbol; Acc: HGNC: 19666] |
| SYTL2 | + | synaptotagmin like 2 [Source: HGNC Symbol; Acc: HGNC: 15585] |
| TBPL1 | + | TATA-box binding protein like 1 [Source: HGNC Symbol; Acc: HGNC: 11589] |
| TFF3 | + | trefoil factor 3 [Source: HGNC Symbol; Acc: HGNC: 11757] |
| TRIM29 | − | tripartite motif containing 29 [Source: HGNC Symbol; Acc: HGNC: 17274] |
| TUBB2A | + | tubulin beta 2A class IIa [Source: HGNC Symbol; Acc: HGNC: 12412] |
| YIPF1 | + | Yip1 domain family member 1 [Source: HGNC Symbol; Acc: HGNC: 25231] |
| ZNF516 | − | zinc finger protein 516 [Source: HGNC Symbol; Acc: HGNC: 28990] |

Cancer population S6

| | | |
|---|---|---|
| CCL2 | + | C-C motif chemokine ligand 2 [Source: HGNC Symbol; Acc: HGNC: 10618] |
| CFB | + | complement factor B [Source: HGNC Symbol; Acc: HGNC: 1037] |
| CFTR | + | cystic fibrosis transmembrane conductance regulator [Source: HGNC Symbol; Acc: HGNC: 1884] |
| CXCL2 | + | C-X-C motif chemokine ligand 2 [Source: HGNC Symbol; Acc: HGNC: 4603] |
| IFI16 | + | interferon gamma inducible protein 16 [Source: HGNC Symbol; Acc: HGNC: 5395] |
| LCN2 | + | lipocalin 2 [Source: HGNC Symbol; Acc: HGNC: 6526] |
| LTF | + | lactotransferrin [Source: HGNC Symbol; Acc: HGNC: 6720] |
| LXN | + | latexin [Source: HGNC Symbol; Acc: HGNC: 13347] |
| TFRC | + | transferrin receptor [Source: HGNC Symbol; Acc: HGNC: 11763] |

Cancer population S7

| | | |
|---|---|---|
| ACTG2 | − | actin, gamma 2, smooth muscle, enteric [Source: HGNC Symbol; Acc: HGNC: 145] |
| ACTN1 | − | actinin alpha 1 [Source: HGNC Symbol; Acc: HGNC: 163] |
| ADAMTS1 | − | ADAM metallopeptidase with thrombospondin type 1 motif 1 [Source: HGNC Symbol; Acc: HGNC: 217] |
| ANPEP | − | alanyl aminopeptidase, membrane [Source: HGNC Symbol; Acc: HGNC: 500] |
| ARMCX1 | − | armadillo repeat containing, X-linked 1 [Source: HGNC Symbol; Acc: HGNC: 18073] |
| AZGP1 | − | alpha-2-glycoprotein 1, zinc-binding [Source: HGNC Symbol; Acc: HGNC: 910] |
| C7 | − | complement C7 [Source: HGNC Symbol; Acc: HGNC: 1346] |
| CD44 | − | CD44 molecule (Indian blood group) [Source: HGNC Symbol; Acc: HGNC: 1681] |
| CHRDL1 | − | chordin like 1 [Source: HGNC Symbol; Acc: HGNC: 29861] |
| CNN1 | − | calponin 1 [Source: HGNC Symbol; Acc: HGNC: 2155] |
| CRISPLD2 | − | cysteine rich secretory protein LCCL domain containing 2 [Source: HGNC Symbol; Acc: HGNC: 25248] |
| CSRP1 | − | cysteine and glycine rich protein 1 [Source: HGNC Symbol; Acc: HGNC: 2469] |
| CYP27A1 | − | cytochrome P450 family 27 subfamily A member 1 [Source: HGNC Symbol; Acc: HGNC: 2605] |
| CYR61 | − | cysteine rich angiogenic inducer 61 [Source: HGNC Symbol; Acc: HGNC: 2654] |
| DES | − | desmin [Source: HGNC Symbol; Acc: HGNC: 2770] |
| EGR1 | − | early growth response 1 [Source: HGNC Symbol; Acc: HGNC: 3238] |
| ETS2 | − | ETS proto-oncogene 2, transcription factor [Source: HGNC Symbol; Acc: HGNC: 3489] |
| F5 | + | coagulation factor V [Source: HGNC Symbol; Acc: HGNC: 3542] |
| FBLN1 | − | fibulin 1 [Source: HGNC Symbol; Acc: HGNC: 3600] |
| FERMT2 | − | fermitin family member 2 [Source: HGNC Symbol; Acc: HGNC: 15767] |
| FHL2 | − | four and a half LIM domains 2 [Source: HGNC Symbol; Acc: HGNC: 3703] |
| FLNA | − | filamin A [Source: HGNC Symbol; Acc: HGNC: 3754] |
| FXYD6 | − | FXYD domain containing ion transport regulator 6 [Source: HGNC Symbol; Acc: HGNC: 4030] |
| FZD7 | − | frizzled class receptor 7 [Source: HGNC Symbol; Acc: HGNC: 4045] |
| ITGA5 | − | integrin subunit alpha 5 [Source: HGNC Symbol; Acc: HGNC: 6141] |
| ITM2C | − | integral membrane protein 2C [Source: HGNC Symbol; Acc: HGNC: 6175] |
| JAM3 | − | junctional adhesion molecule 3 [Source: HGNC Symbol; Acc: HGNC: 15532] |
| JUN | − | Jun proto-oncogene, AP-1 transcription factor subunit [Source: HGNC Symbol; Acc: HGNC: 6204] |
| KHDRBS3 | + | KH RNA binding domain containing, signal transduction associated 3 [Source: HGNC Symbol; Acc: HGNC: 18117] |
| LMOD1 | − | leiomodin 1 [Source: HGNC Symbol; Acc: HGNC: 6647] |
| LPHN2 | − | NA |
| MT1M | − | metallothionein 1M [Source: HGNC Symbol; Acc: HGNC: 14296] |
| MYH11 | − | myosin heavy chain 11 [Source: HGNC Symbol; Acc: HGNC: 7569] |
| MYL9 | − | myosin light chain 9 [Source: HGNC Symbol; Acc: HGNC: 15754] |
| NFIL3 | − | nuclear factor, interleukin 3 regulated [Source: HGNC Symbol; Acc: HGNC: 7787] |
| PARM1 | − | prostate androgen-regulated mucin-like protein 1 [Source: HGNC Symbol; Acc: HGNC: 24536] |
| PCP4 | − | Purkinje cell protein 4 [Source: HGNC Symbol; Acc: HGNC: 8742] |
| PDK4 | − | pyruvate dehydrogenase kinase 4 [Source: HGNC Symbol; Acc: HGNC: 8812] |
| PLAGL1 | − | PLAG1 like zinc finger 1 [Source: HGNC Symbol; Acc: HGNC: 9046] |
| RAB27A | − | RAB27A, member RAS oncogene family [Source: HGNC Symbol; Acc: HGNC: 9766] |
| SERPINF1 | − | serpin family F member 1 [Source: HGNC Symbol; Acc: HGNC: 8824] |
| SNAI2 | − | snail family transcriptional repressor 2 [Source: HGNC Symbol; Acc: HGNC: 11094] |
| SORBS1 | − | sorbin and SH3 domain containing 1 [Source: HGNC Symbol; Acc: HGNC: 14565] |
| SPARCL1 | − | SPARC like 1 [Source: HGNC Symbol; Acc: HGNC: 11220] |
| SPOCK3 | − | SPARC/osteonectin, cwcv and kazal like domains proteoglycan 3 [Source: HGNC Symbol; Acc: HGNC: 13565] |
| SYNM | − | synemin [Source: HGNC Symbol; Acc: HGNC: 24466] |
| TAGLN | − | transgelin [Source: HGNC Symbol; Acc: HGNC: 11553] |
| TCEAL2 | − | transcription elongation factor A like 2 [Source: HGNC Symbol; Acc: HGNC: 29818] |
| TGFB3 | − | transforming growth factor beta 3 [Source: HGNC Symbol; Acc: HGNC: 11769] |
| TPM2 | − | tropomyosin 2 (beta) [Source: HGNC Symbol; Acc: HGNC: 12011] |
| VCL | − | vinculin [Source: HGNC Symbol; Acc: HGNC: 12665] |

Cancer population S7

| | | |
|---|---|---|
| ABCC4 | − | ATP binding cassette subfamily C member 4 [Source: HGNC Symbol; Acc: HGNC: 55] |
| ACAT2 | − | acetyl-CoA acetyltransferase 2 [Source: HGNC Symbol; Acc: HGNC: 94] |
| ARHGEF6 | + | Rac/Cdc42 guanine nucleotide exchange factor 6 [Source: HGNC Symbol; Acc: HGNC: 685] |
| ATP8A1 | − | ATPase phospholipid transporting 8A1 [Source: HGNC Symbol; Acc: HGNC: 13531] |
| AXL | + | AXL receptor tyrosine kinase [Source: HGNC Symbol; Acc: HGNC: 905] |
| CANT1 | − | calcium activated nucleotidase 1 [Source: HGNC Symbol; Acc: HGNC: 19721] |
| CD83 | + | CD83 molecule [Source: HGNC Symbol; Acc: HGNC: 1703] |
| CDH1 | − | cadherin 1 [Source: HGNC Symbol; Acc: HGNC: 1748] |
| COL15A1 | + | collagen type XV alpha 1 chain [Source: HGNC Symbol; Acc: HGNC: 2192] |
| DCXR | − | dicarbonyl and L-xylulose reductase [Source: HGNC Symbol; Acc: HGNC: 18985] |
| DHCR24 | − | 24-dehydrocholesterol reductase [Source: HGNC Symbol; Acc: HGNC: 2859] |
| DHRS7 | − | dehydrogenase/reductase 7 [Source: HGNC Symbol; Acc: HGNC: 21524] |
| DPYSL3 | + | dihydropyrimidinase like 3 [Source: HGNC Symbol; Acc: HGNC: 3015] |
| EPB41L3 | + | erythrocyte membrane protein band 4.1 like 3 [Source: HGNC Symbol; Acc: HGNC: 3380] |
| FAM174B | − | family with sequence similarity 174 member B [Source: HGNC Symbol; Acc: HGNC: 34339] |
| FAM189A2 | − | family with sequence similarity 189 member A2 [Source: HGNC Symbol; Acc: HGNC: 24820] |
| FBN1 | + | fibrillin 1 [Source: HGNC Symbol; Acc: HGNC: 3603] |
| FCHSD2 | + | FCH and double SH3 domains 2 [Source: HGNC Symbol; Acc: HGNC: 29114] |
| FHL1 | + | four and a half LIM domains 1 [Source: HGNC Symbol; Acc: HGNC: 3702] |
| FKBP4 | − | FK506 binding protein 4 [Source: HGNC Symbol; Acc: HGNC: 3720] |
| FOXA1 | − | forkhead box A1 [Source: HGNC Symbol; Acc: HGNC: 5021] |
| FXYD5 | + | FXYD domain containing ion transport regulator 5 [Source: HGNC Symbol; Acc: HGNC: 4029] |
| GNAO1 | + | G protein subunit alpha o1 [Source: HGNC Symbol; Acc: HGNC: 4389] |
| GOLM1 | − | golgi membrane protein 1 [Source: HGNC Symbol; Acc: HGNC: 15451] |
| GPX3 | + | glutathione peroxidase 3 [Source: HGNC Symbol; Acc: HGNC: 4555] |
| GTF3C1 | − | general transcription factor IIIC subunit 1 [Source: HGNC Symbol; Acc: HGNC: 4664] |
| HPN | − | hepsin [Source: HGNC Symbol; Acc: HGNC: 5155] |
| IFI16 | + | interferon gamma inducible protein 16 [Source: HGNC Symbol; Acc: HGNC: 5395] |
| IRAK3 | + | interleukin 1 receptor associated kinase 3 [Source: HGNC Symbol; Acc: HGNC: 17020] |
| ITGA5 | + | integrin subunit alpha 5 [Source: HGNC Symbol; Acc: HGNC: 6141] |
| KIF5C | − | kinesin family member 5C [Source: HGNC Symbol; Acc: HGNC: 6325] |
| KLK3 | − | kallikrein related peptidase 3 [Source: HGNC Symbol; Acc: HGNC: 6364] |
| LAPTM5 | + | lysosomal protein transmembrane 5 [Source: HGNC Symbol; Acc: HGNC: 29612] |
| MAP7 | − | microtubule associated protein 7 [Source: HGNC Symbol; Acc: HGNC: 6869] |
| MBOAT2 | − | membrane bound O-acyltransferase domain containing 2 [Source: HGNC Symbol; Acc: HGNC: 25193] |
| MFAP4 | + | microfibrillar associated protein 4 [Source: HGNC Symbol; Acc: HGNC: 7035] |
| MFGE8 | + | milk fat globule-EGF factor 8 protein [Source: HGNC Symbol; Acc: HGNC: 7036] |
| MIOS | − | meiosis regulator for oocyte development [Source: HGNC Symbol; Acc: HGNC: 21905] |
| MLPH | − | melanophilin [Source: HGNC Symbol; Acc: HGNC: 29643] |
| MMP2 | + | matrix metallopeptidase 2 [Source: HGNC Symbol; Acc: HGNC: 7166] |
| MYO5C | − | myosin VC [Source: HGNC Symbol; Acc: HGNC: 7604] |
| NEDD4L | − | neural precursor cell expressed, developmentally down-regulated 4-like, E3 ubiquitin protein ligase |
| | | [Source: HGNC Symbol; Acc: HGNC: 7728] |
| PART1 | − | prostate androgen-regulated transcript 1 (non-protein coding) [Source: HGNC Symbol; Acc: HGNC: 17263] |
| PARVA | + | parvin alpha [Source: HGNC Symbol; Acc: HGNC: 14652] |
| PDIA5 | − | protein disulfide isomerase family A member 5 [Source: HGNC Symbol; Acc: HGNC: 24811] |
| PIGH | − | phosphatidylinositol glycan anchor biosynthesis class H [Source: HGNC Symbol; Acc: HGNC: 8964] |
| PLEKHO1 | + | pleckstrin homology domain containing O1 [Source: HGNC Symbol; Acc: HGNC: 24310] |
| PLSCR4 | + | phospholipid scramblase 4 [Source: HGNC Symbol; Acc: HGNC: 16497] |
| PMEPA1 | − | prostate transmembrane protein, androgen induced 1 [Source: HGNC Symbol; Acc: HGNC: 14107] |
| PRSS8 | − | protease, serine 8 [Source: HGNC Symbol; Acc: HGNC: 9491] |
| RFTN1 | + | raftlin, lipid raft linker 1 [Source: HGNC Symbol; Acc: HGNC: 30278] |
| SAMD4A | + | sterile alpha motif domain containing 4A [Source: HGNC Symbol; Acc: HGNC: 23023] |
| SAMSN1 | + | SAM domain, SH3 domain and nuclear localization signals 1 [Source: HGNC Symbol; Acc: HGNC: 10528] |
| SEC23B | − | Sec23 homolog B, coat complex II component [Source: HGNC Symbol; Acc: HGNC: 10702] |
| SERPINF1 | + | serpin family F member 1 [Source: HGNC Symbol; Acc: HGNC: 8824] |
| SLC43A1 | − | solute carrier family 43 member 1 [Source: HGNC Symbol; Acc: HGNC: 9225] |
| SPDEF | − | SAM pointed domain containing ETS transcription factor [Source: HGNC Symbol; Acc: HGNC: 17257] |
| SPINT2 | − | serine peptidase inhibitor, Kunitz type 2 [Source: HGNC Symbol; Acc: HGNC: 11247] |
| STEAP4 | − | STEAP4 metalloreductase [Source: HGNC Symbol; Acc: HGNC: 21923] |
| TMPRSS2 | − | transmembrane protease, serine 2 [Source: HGNC Symbol; Acc: HGNC: 11876] |
| TRPM8 | − | transient receptor potential cation channel subfamily M member 8 [Source: HGNC Symbol; Acc: HGNC: 17961] |
| TSPAN1 | − | tetraspanin 1 [Source: HGNC Symbol; Acc: HGNC: 20657] |
| VCAM1 | + | vascular cell adhesion molecule 1 [Source: HGNC Symbol; Acc: HGNC: 12663] |
| WIPF1 | + | WAS/WASL interacting protein family member 1 [Source: HGNC Symbol; Acc: HGNC: 12736] |
| XBP1 | − | X-box binding protein 1 [Source: HGNC Symbol; Acc: HGNC: 12801] |
| ZYX | + | zyxin [Source: HGNC Symbol; Acc: HGNC: 13200] |

The present invention shall now be further described with reference to the following examples, which are present for the purposes of illustration only and are not to be construed as being limiting on invention.

### EXAMPLES

Prostate cancer lacks a robust classification framework causing significant problem in its clinical management. Hierarchical cluster analysis, k-means clustering and iCluster are commonly used unsupervised learning methods for the analysis of single or multiplatform genomic data from prostate and other cancers. Unfortunately, these approaches ignore the fundamentally heterogeneous composition of individual cancer samples. The present inventors use an unsupervised learning model called Latent Process Decomposition (LPD), which can handle heterogeneity within cancer samples, to provide critical insights into the structure of prostate cancer transcriptome datasets. The inventors show that the poor clinical outcome in prostate cancer is dependent on the proportion of cancer containing a signature referred to as DESNT and present a nomogram for using DESNT in clinical management. The inventors identify at least three new clinically and/or genetically distinct subtypes of prostate cancer. The results highlight the importance of devising and using more sophisticated approaches for the analysis of single and multiplatform genomic datasets from all human cancer types.

Unsupervised analysis of prostate cancer transcriptome profiles using the above approaches failed to identify robust disease categories that have distinct clinical outcomes^{7,8}. Noting that prostate cancer samples derived from genome wide studies frequently harbour multiple cancer lineages, and often have heterogeneous compositions⁹⁻¹², the inventors applied an unsupervised learning method called Latent Process Decomposition (LPD)¹³. The inventors had previously used Latent Process Decomposition: (i) to confirm the presence of the basal and ERBB2 overexpressing subtypes in breast cancer transcriptome datasets¹⁴; (ii) to demonstrate that data from the MammaPrint breast cancer recurrence assay would be optimally analyzed using four separate prognostic categories¹⁴; and (iii) to show that patients with advanced prostate cancer can be stratified into two clinically distinct categories based on expression profiles in blood¹⁵. LPD (closely related to Latent Dirichlet Allocation¹⁶) is a mixed membership model in which the expression profile for a cancer is represented as a combination of underlying latent processes. Each latent process is considered as an underlying functional state or the expression profile of a particular component of the cancer. A given sample can be represented over a number of these underlying functional states, or just one such state. The appropriate number of processes to use (the model complexity) is determined using the LPD algorithm by maximising the probability of the model given the data.

The application of LPD to prostate cancer transcriptome datasets led to the discovery of an expression pattern, called DESNT, that was observed in all prostate cancer datasets examined¹⁷. Cancers were assigned as DESNT when this pattern was more common than any other signature, and designation of a patients as having DESNT cancer predicted poor outcome independently of other clinical parameters including Gleason sum, Clinical stage and PSA. In the current paper the inventors test a key prediction of the DESNT cancer model, and use LPD to develop a new prostate cancer framework.

Results

Presence of DESNT Signature Predicts Poor Clinical Outcome.

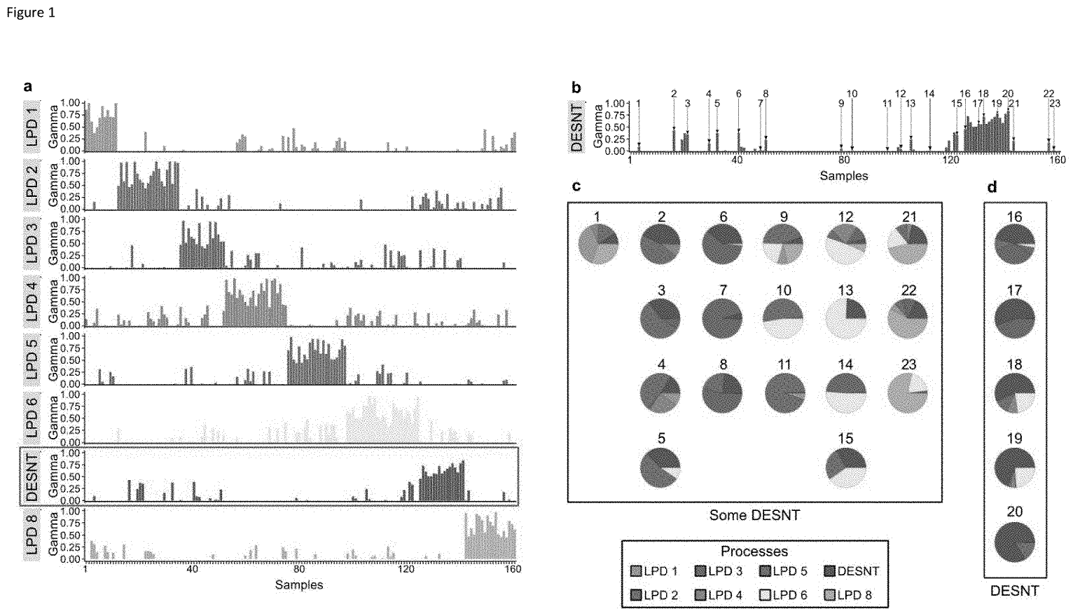

In previous studies optimal decomposition of expression microarray datasets was performed using between 3 and 8 underlying processes¹⁷. An illustration of the decomposition of the MSKCC dataset⁸ into 8 processes is shown in FIG. 1a. LPD Process 7 illustrates the percentage of the DESNT expression signature identified in each sample, with individual cancer being assigned as a “DESNT cancer” when the DESNT signature was the most abundant as shown in FIGS. 1b and 1d. Based on PSA failure patients with DESNT cancers always exhibited poorer outcome relative to other cancers in the same dataset¹⁷. The implication is that it is the presence of regions of cancer containing the DESNT signature that conferred poor outcome. If this model is correct the inventors would predict that cancers containing smaller contribution of DESNT signature, such as those shown in FIG. 1c for the MSKCC dataset, should also exhibit poorer outcome.

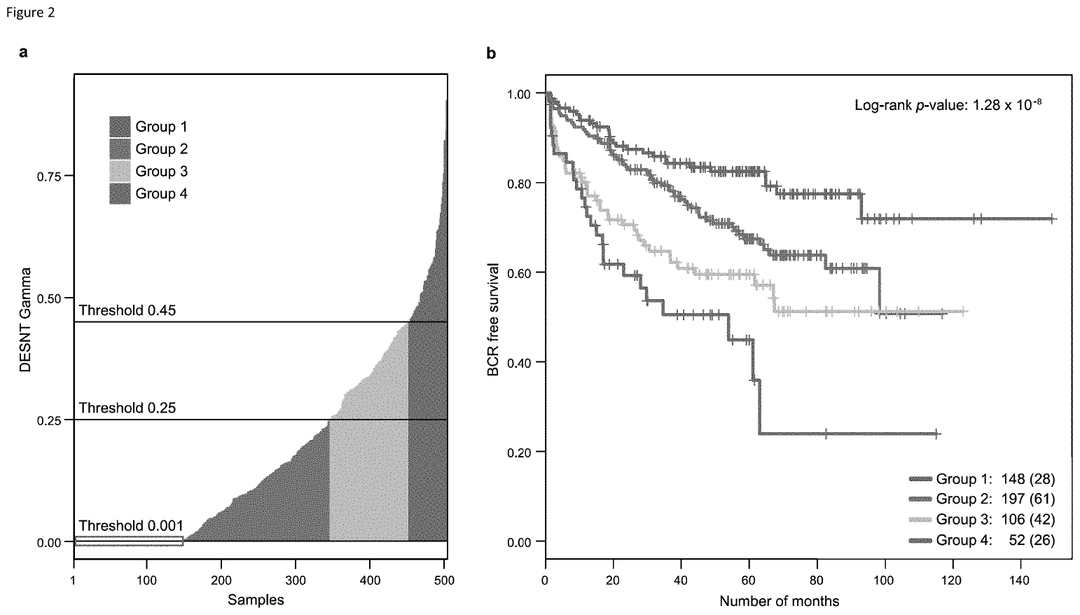

To increase the power to test this prediction the inventors combined data from cancers from the MSKCC⁸, CancerMap¹⁷, Stephenson¹⁸, and CamCap⁷ (n=503) studies. Treating the proportion of expression assigned to the DESNT process (Gamma) as a continuous variable the inventors found that there was a significant association with PSA recurrence (P=8.96×10⁻¹⁴, HR=1.52, 95% C1=[1.36, 1.7], Cox proportional hazard regression model). Outcome became worse as Gamma increased. This is illustrated by dividing the cancers into four groups based on the proportion of the DESNT process present (FIG. 2a). PSA failure free survival is then as follows (FIG. 2b): (i) no DESNT cancer, 82.5% at 60 months; (ii) less than 0.25 Gamma, 67.4% at 60 months; (iii) 0.25 to 0.45 Gamma, 59.5% at 60 months and (iv) >0.45 Gamma, 44.9% at 60 months. Overall 70.6% of cancers contained at least some DESNT cancer (FIG. 2a).

Nomogram for DESNT Predicting PSA Failure

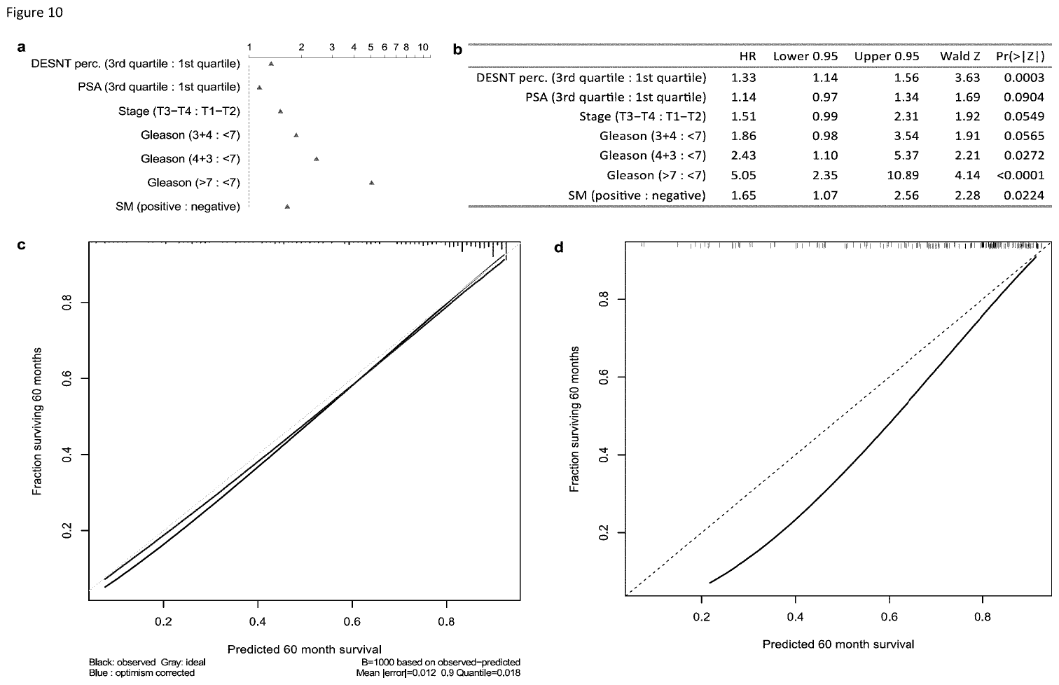

The proportion of DESNT cancer was combined with other clinical variables (Gleason grade, PSA levels, pathological stage and the surgical margins status) in a Cox proportional hazards model and fitted to a combined dataset of 318 cancers; CamCap cancers (n=185) were used for external validation. DESNT Gamma was an independent predictor of worse clinical outcome (P=3×10⁻⁴, HR=1.33, 95% C1=[1.14, 1.56]) along with Gleason grade=4+3 (P=2.7×10⁻², HR=2.43, 95% C1=[1.10, 5.37]), Gleason grade>7 (P<1×10⁻⁴, HR=5.05, 95% C1=[2.35, 10.89]), and positive surgical margins (P=2.24×10⁻², HR=1.65, 95% C1=[1.07, 2.56]) (FIG. 10). PSA level as a predictor and pathological stage were below the threshold of statistical significance (P=0.09, HR=1.14, 95% C1=[0.97, 1.34]) and (P=5.49×10⁻², HR=1.51, 95% C1=[0.99, 2.31]) respectively. At internal validation, the Cox model obtained a bootstrap-corrected C-index of 0.747, and at external validation a C-index of 0.795. Using this model the inventors have devised a nomogram for use of DESNT cancer together with clinical variables (FIGS. 3 and 10) to predict the risk of biochemical recurrence at 1, 3, 5 and 7 years following prostatectomy.

LPD Algorithm for Detecting the Presence of DESNT Cancer in Individual Samples.

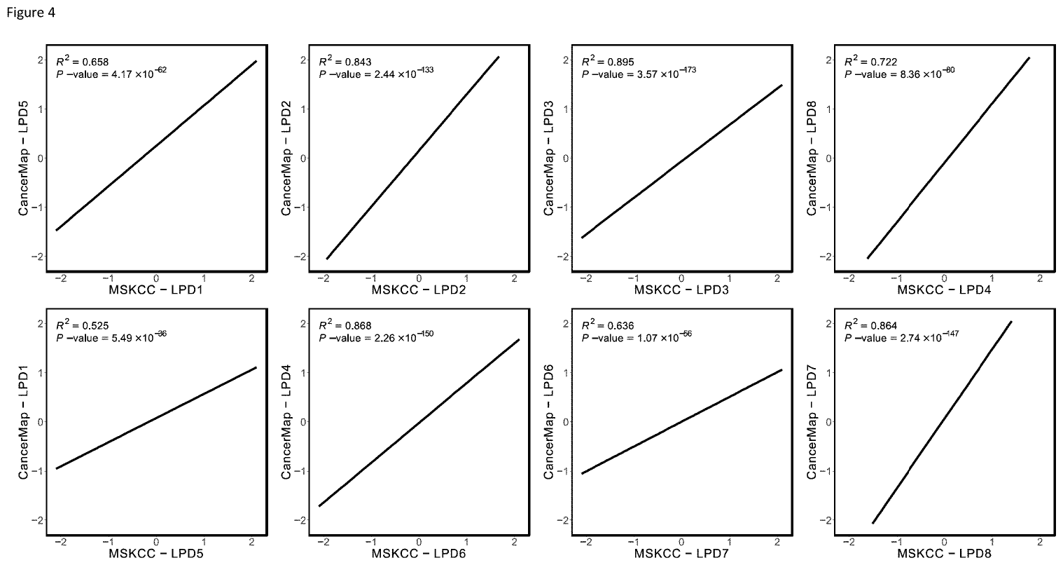

The ability of LPD to detect structure in different datasets, with optimal decompositions varying between 3 and 8 underlying processes¹⁷, is likely to be dependent on sample size, cohort composition and data quality. When the inventors examined the two datasets that were analysed using 8 underlying processes (MSKCC and CancerMap) the inventors noted a striking relationship: based on correlations of expression profiles; all eight of the LPD processes appeared to be common (FIG. 4; R²>0.5). To provide a more consistent classification framework where the number of classes did not vary between datasets the inventors therefore used the MSKCC dataset and its decomposition into 8 distinct processes as a reference for identifying categories of human prostate cancer.

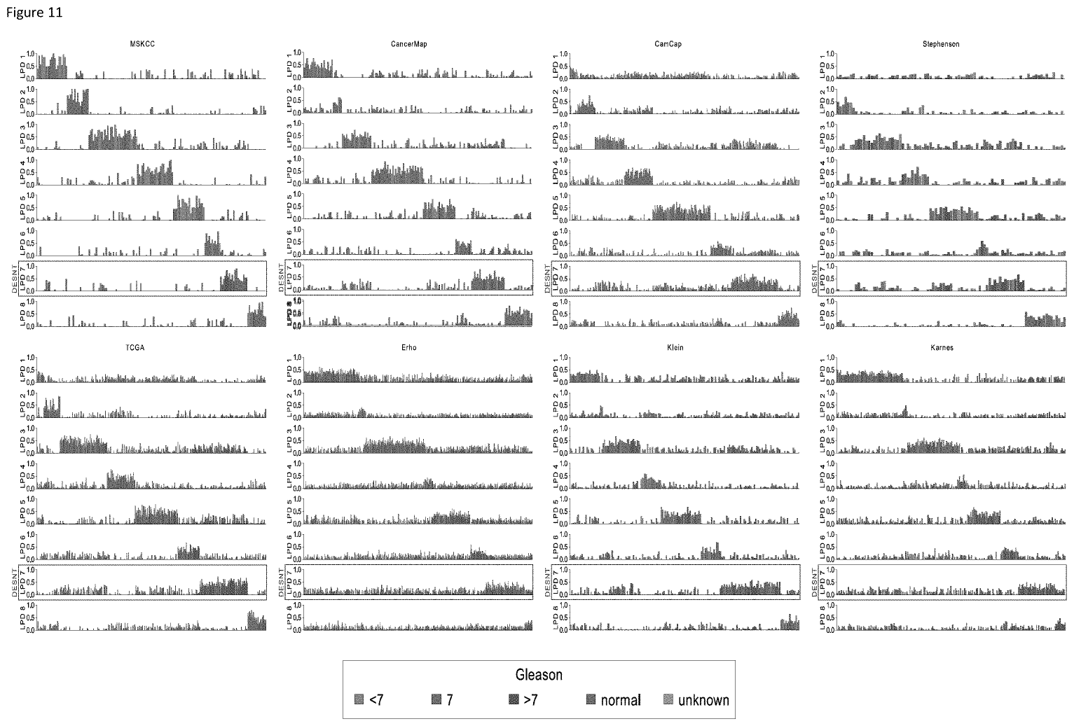

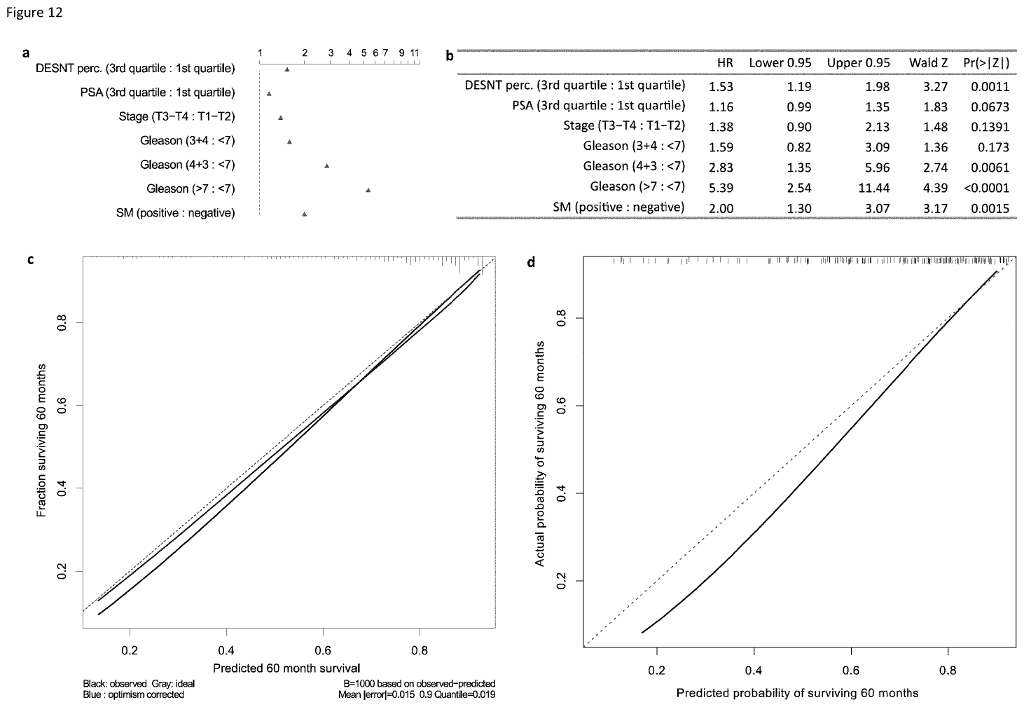

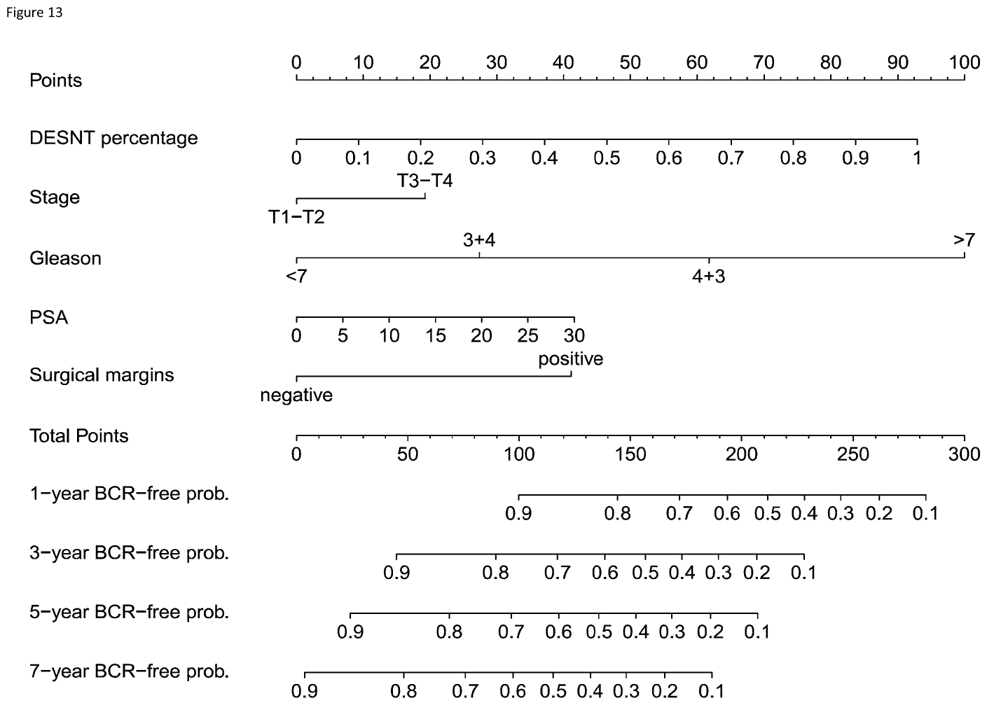

The inventors developed a variant of LPD called OAS-LPD (One Added Sample-LPD) where data from a single additional cancer could be decomposed into processes, following normalisation, without repeating the entire computing-intensive LPD procedure. LPD model parameters¹³μ_{gk}, σ²_{gk} and a were first derived by decomposition of the MSKCC dataset into 8 processes. These parameters can then be used as the basis for decomposition of data from additional single samples, selected from a dataset under examination, or from a patient undergoing assessment in the clinic. To test this procedure, the inventors applied OAS-LPD individually to cancers from MSKCC⁸, CancerMap¹⁷, Stephenson¹⁸, and CamCap⁷ (FIG. 11) and repeated Cox regression analysis and nomogram construction. DESNT Gamma (P=1.1×10⁻³, HR=1.53, 95% CI=[1.19, 1.98]), Gleason=4+3 (P=6.1×10⁻³, HR=2.83, 95% CI=[1.35, 5.96]), Gleason>7 (P<1×10⁻⁴, HR=5.39, 95% CI=[2.54, 11.44]) and surgical margin status (P=1.5×10⁻³, HR=2.00, 95% CI=[1.30, 3.07]) remained independent predictors of clinical outcome (FIG. 12). Notably the performance of the Cox model (internal validation C-index=0.742; external validation C-index=0.786) was not significantly different to that of the model in FIG. 10 (train dataset Z=−0.65, two-tailed P=0.52; validation dataset Z=0.89, two-tailed P=0.38; U-statistic¹⁹) and the nomogram (FIG. 13) had almost an identical presentation of parameters to that shown in FIG. 3.

New Categories of Human Prostate Cancer

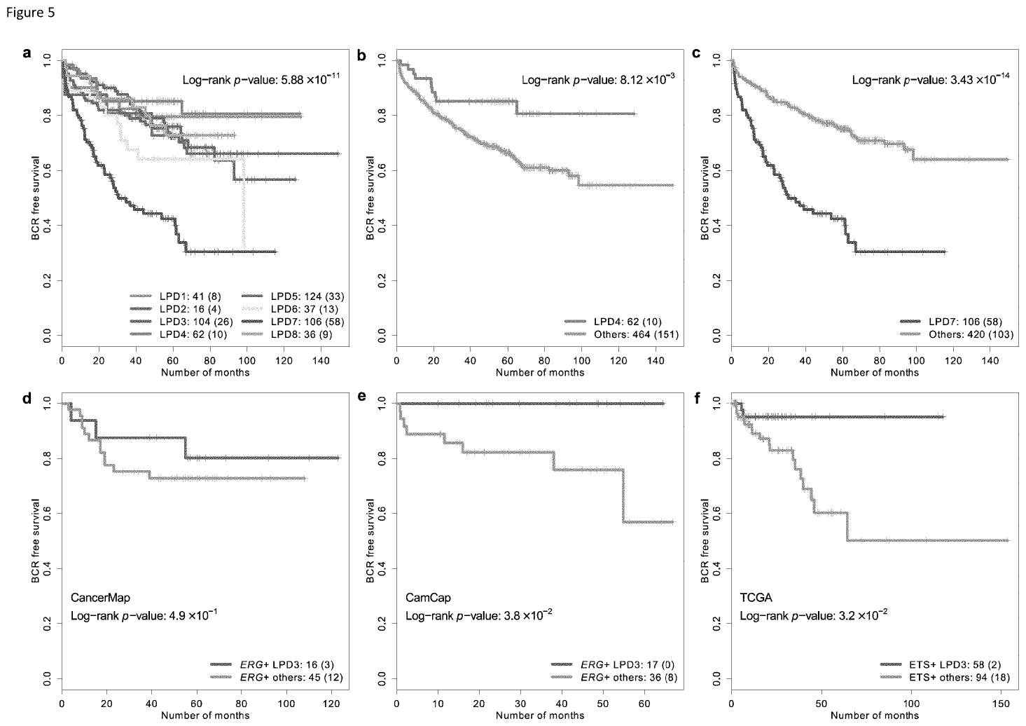

The inventors wished to determine whether particular LPD processes were associated with clinical or molecular features indicating that they represented distinct categories of human prostate cancer. LPD2, LPD4 and LPD8 more frequently contained normal prostate samples (FIG. 11 and Table 6). When datasets with linked clinical data were combined (FIG. 5a-c) cancers assigned to LPD7 had worse outcome (DESNT, P=3.43×10⁻¹⁴, log-rank test) while those assigned to LPD4 had improved outcome (S4, P=8.12×10⁻³, log-rank test) as judged by PSA failure. Within the LPD3 subgroup cancers with ERG-alterations also exhibited better outcome (P<0.05; log-rank test) in two of three datasets (FIG. 5d-f).

TABLE 6

| | TCGA | | | CancerMap | | | CamCap | | |
|---|---|---|---|---|---|---|---|---|---|
| | Benign | Primary | χ² P-val | Benign | Primary | χ² P-val | Benign | Primary | χ² P-val |
| LPD1 | 0 | 11 | 0.466092 | 9 | 13 | 0.195522 | 2 | 7 | 1 |
| LPD2 | 15 | 12 | 7.89E−13 | 4 | 3 | 0.165632 | 17 | 4 | 1.21E−08 |
| LPD3 | 1 | 76 | 0.00335 | 0 | 22 | 0.004958 | 0 | 36 | 0.000302 |
| LPD4 | 11 | 35 | 0.00957 | 16 | 23 | 0.044844 | 30 | 5 | 5.02E−17 |
| LPD5 | 0 | 70 | 0.001781 | 1 | 24 | 0.010098 | 0 | 71 | 1.75E−08 |
| LPD6 | 1 | 35 | 0.149512 | 5 | 7 | 0.404231 | 6 | 19 | 0.993199 |
| LPD7 | 0 | 79 | 0.000687 | 1 | 24 | 0.010098 | 0 | 57 | 1.20E−06 |
| LPD8 | 15 | 15 | 3.60E−11 | 11 | 10 | 0.012093 | 18 | 8 | 4.94E−07 |

| | MSKCC | | | Stephenson | | |
|---|---|---|---|---|---|---|
| | Benign | Primary | χ² P-val | Benign | Primary | χ² P-val |
| LPD1 | 3 | 18 | 0.852347 | — | — | — |
| LPD2 | 12 | 3 | 6.30E−10 | 3 | 4 | 0.050471 |
| LPD3 | 0 | 34 | 0.004501 | 0 | 18 | 0.166692 |
| LPD4 | 6 | 19 | 0.584004 | 1 | 10 | 1 |
| LPD5 | 0 | 22 | 0.037682 | 0 | 19 | 0.146293 |
| LPD6 | 0 | 11 | 0.225693 | 0 | 4 | 1 |
| LPD7 | 0 | 19 | 0.061832 | 0 | 14 | 0.276438 |
| LPD8 | 8 | 5 | 0.000112 | 7 | 9 | 0.000149 |

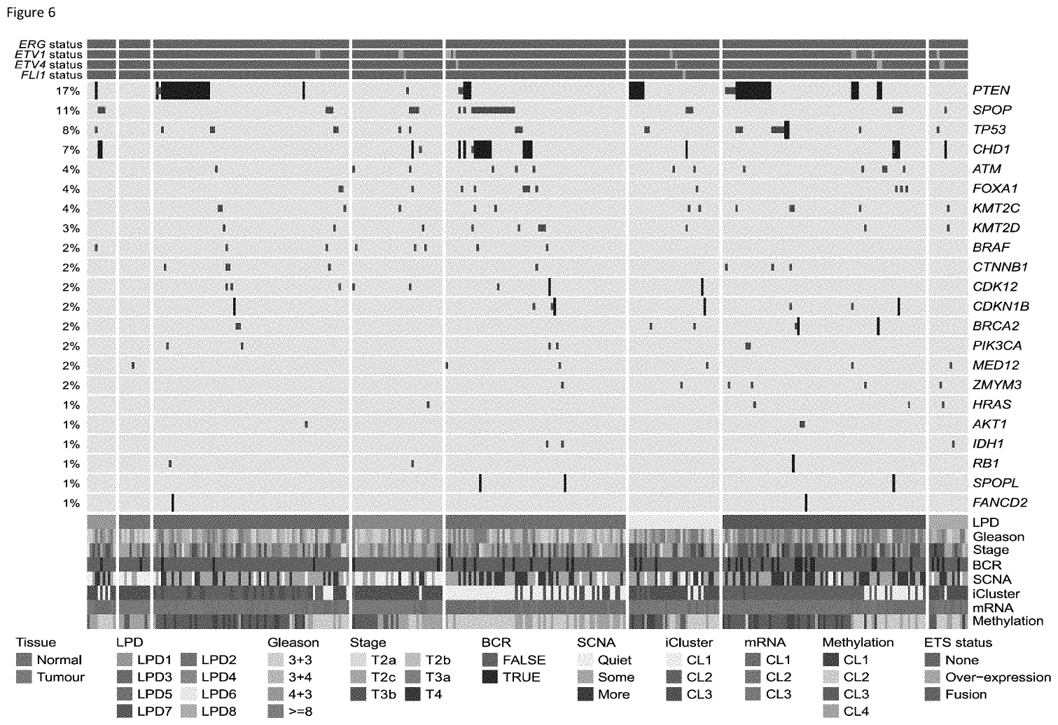

Examining the distribution of genetic alterations in the decomposition of the TGCA dataset²⁰ (FIG. 6), LPD3 (Cancers where LPD3 has the highest Gamma are referred to as S3-cancers; other assignments are LPD1=S1, LPD2=S2, LPD4=S4, LPD5=5, LPD6=S6, LPD7=DESNT, and LPD8=S8) had over-representation of ETS and PTEN gene alterations, and under-representation of CDH1 and SPOP gene alterations (P<0.05, χ² test, Table 7). S5 cancers exhibited exactly the reverse pattern of genetic alteration: there was under-repression of ETS and PTEN gene alterations and over-representation SPOP and CHD1 gene changes (Table 7). DESNT cancers exhibited overrepresentation of ETS and PTEN gene alterations. The statistically different distribution of ETS-gene alteration in S3, S5 and DESNT observed in the TGCA dataset were confirmed in the CamCap and CancerMap dataset (Table 7). In summary the inventors have identified three additional prostate cancer categories that have altered genetic and/or clinical associations: S3, S4 and S5 (FIG. 7).

TABLE 7
Correlation of OAS-LPD subgroups with genetic alterations in The Cancer Genome
Atlas Dataset. Statistically significant differences are highlighted in grey.

| | TCGA | | | CancerMap | | | CamCap | | |
|---|---|---|---|---|---|---|---|---|---|
| | ETS− | ETS+ | χ² P-val | ERG− | ERG+ | χ² P-val | ERG− | ERG+ | χ² P-val |
| LPD1 | 8 | 3 | 0.05758 | 13 | 4 | 0.08512 | 0 | 3 | 0.2349 |
| LPD2 | 4 | 8 | 0.827 | 3 | 3 | 1 | 0 | 2 | 0.4671 |
| LPD3 | 9 | 67 | 1.45E−08 | 5 | 15 | 0.00977 | 4 | 17 | 0.00299 |
| LPD4 | 14 | 21 | 1 | 14 | 15 | 0.6193 | 1 | 2 | 0.9869 |
| LPD5 | 65 | 5 | 2.20E−16 | 19 | 1 | 0.00018 | 34 | 0 | 1.15E−11 |
| LPD6 | 13 | 22 | 0.802 | 5 | 5 | 1 | 2 | 4 | 0.6572 |
| LPD7 | 13 | 66 | 1.17E−06 | 6 | 15 | 0.02068 | 9 | 24 | 0.00274 |
| LPD8 | 9 | 6 | 0.193 | 8 | 4 | 0.5395 | 4 | 1 | 0.3709 |

| | PTEN | | | SPOP | | | CHD1 | | |
|---|---|---|---|---|---|---|---|---|---|
| | Non-homdel | Homdel | χ² P-val | Non-mut | Mut | χ² P-val | Non-homdel | Homdel | χ² P-val |
| LPD1 | 10 | 1 | 0.8964 | 8 | 3 | 0.2125 | 9 | 2 | 0.3091 |
| LPD2 | 12 | 0 | 0.2839 | 12 | 0 | 0.4356 | 12 | 0 | 0.7561 |
| LPD3 | 55 | 21 | 0.000894 | 73 | 3 | 0.03995 | 76 | 0 | 0.02111 |
| LPD4 | 35 | 0 | 0.01738 | 31 | 4 | 1 | 34 | 1 | 0.6032 |
| LPD5 | 67 | 3 | 0.008304 | 51 | 19 | 4.46E−06 | 57 | 13 | 7.69E−06 |
| LPD6 | 29 | 6 | 0.9026 | 32 | 3 | 0.825 | 34 | 1 | 0.6032 |
| LPD7 | 60 | 19 | 0.01667 | 75 | 4 | 0.07952 | 76 | 3 | 0.4322 |
| LPD8 | 15 | 0 | 0.195 | 14 | 1 | 0.8886 | 14 | 1 | 1 |

Altered Patterns of Gene Expression and DNA Methylation

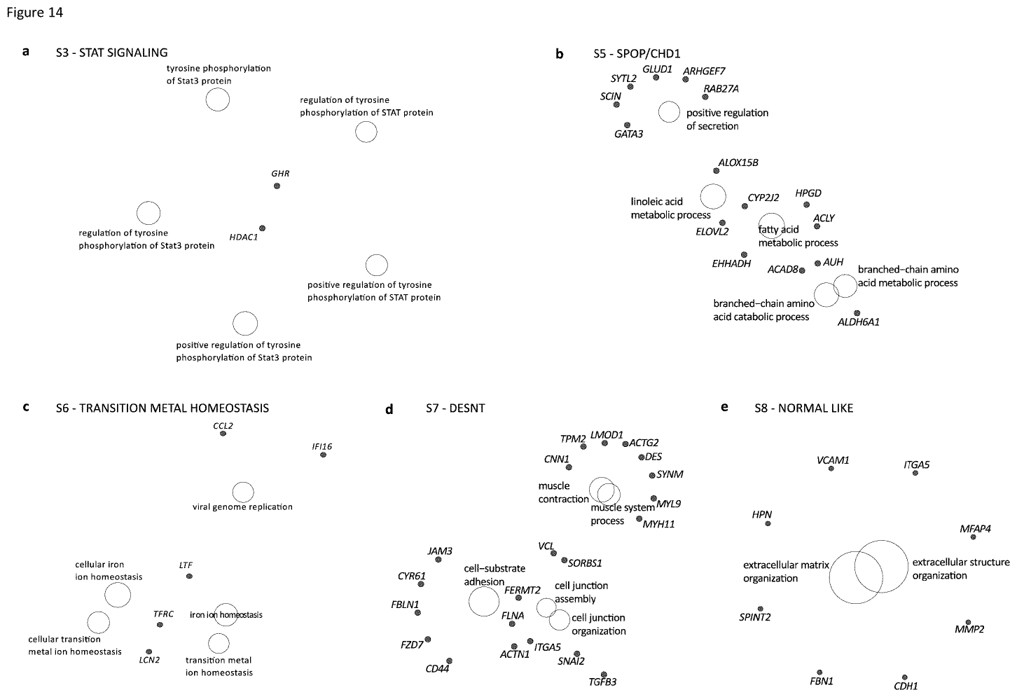

The inventors screened for genes that had significantly altered expression levels (P<0.05 after FDR correction) in each LPD process compared to gene expression levels in all other LPD categories from the same dataset. The inventors then identified genes commonly altered for that process across all 8 datasets (Table 5). Where the LPD process had less than 10 assigned cancers they were not included in the analyses. S3 cancers exhibited 7 commonly overexpressed genes including ERG, GHR and HDAC1. Pathway analysis suggested the involvement of Stat3 gene signalling (FIG. 14a). S5 exhibited 47 significantly overexpressed gene and 13 under-expressed genes. Many of the genes had established roles in fatty acid metabolism and the control of secretion (FIG. 14b). S6-cancers and S8 cancers had failed to exhibit statistically significant changes in genetic alteration or clinical outcome in the current study but did have characteristic altered patterns of gene expression (FIG. 14c,e). The five genes commonly overexpressed in S6 cancers suggested involvement in metal ion homeostasis. 30 genes were overexpressed and 36 genes under expressed in in S8 cancers including several genes involved in extracellular matrix organisation. Cross referencing differential methylation data available for the TOGA dataset with alterations of expression common across all datasets indicated that many expression changes may be explained, at least in part, by changes in DNA methylation (FIG. 7).

49 genes exhibited low expression in DESNT cancers including 20 genes previously identified as associated with this disease category¹⁷. Within prostate some of the 49 genes have restricted expression in stroma (e.g. ITGA5, PCP4, DPYSL3, and FBLN1) indicating that DESNT cancer may be associated with a low stroma content. For two of the clinical series stromal cell contents, as determined by histopathology, were available but there was no overall correlation between stromal content and clinical outcome (log-rank test; CancerMap, P=0.159; CamCap, P=0.261). Cancers assigned as DESNT did however have a significantly lower stromal content compared to non-stromal cancer (Mann

Whitney U test; CancerMap, P=6.7×10⁻³; CamCap p=2.4×10⁻²). The inventors concluded that DESNT cancer represents a subset of the cancers that have low stroma content but that low stroma content does not automatically make a cancer poor prognosis.

DESNT as a Signature of Metastasis.

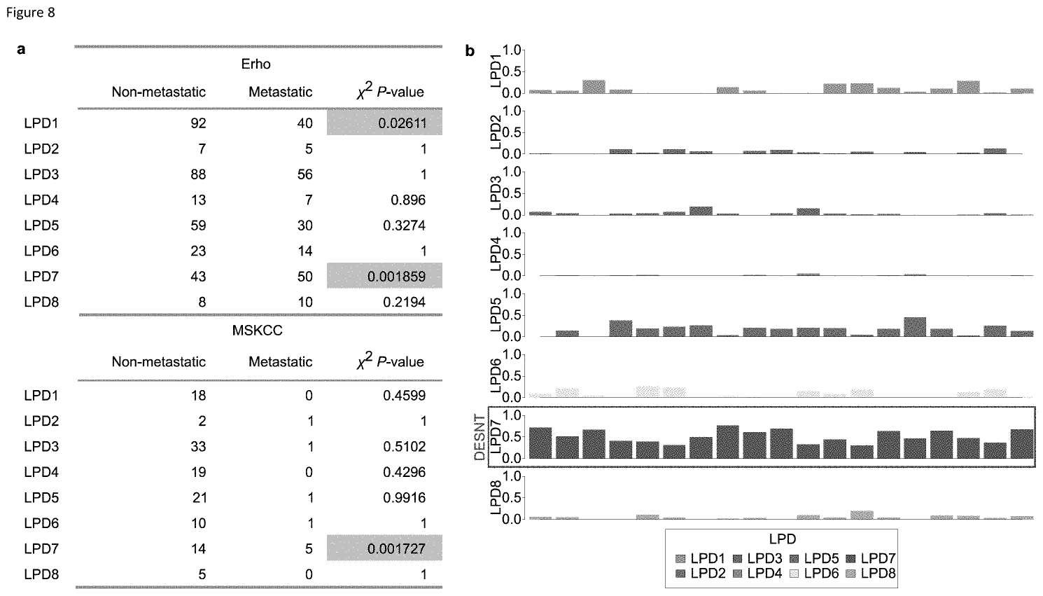

Two of the studied datasets (MSKCC and Erho) (FIG. 11) had publically available annotations indicating that the primary cancers whose expression profiles were examined had progressed to develop metastasis. From 9 cancers developing metastasis in the MSKCC dataset 5 occurred from DESNT cancer (χ²-test, P=1.73×10⁻³) and of 212 cancers developing metastases in the Erho dataset 50 were from DESNT cancers (χ²-test, P=1.86×10⁻³) (FIG. 8a). These studies were based on the definition¹⁷ that DESNT cancers are those in which the DESNT signature is most common. From these studies the inventors concluded that DESNT cancers have an increased risk of developing metastasis, consistent with the higher risk of PSA failure¹⁷. For the Erho dataset membership of S1 was also associated with higher risk of metastasis (FIG. 8a). The MSKCC study additionally reported expression profiles from 19 metastatic cancers. To further examine the relationship between the DESNT cancer signature and metastatic disease the inventors subject expression profiles from each of the metastases to OAS-LPD. In each case the DESNT signature was the most common (FIG. 8b).

To further investigate the underlying nature of DESNT cancer the inventors used the transcriptome profile for each prostate cancer to calculate the status of the 17,697 signatures and pathways annotated in the MSigDB database. The top 20 correlations to proportions of DESNT Gamma are show in Table 8. Notably the 3^{rd} most significant correlation was to genes downregulated in metastatic prostate cancer. The data give addition potential clues to the underlying biology of DESNT cancer including associations with genes altered in ductal breast cancer, in stem cells and during FGFR1 signaling. The correlation to genes whose expression is reactivated following the treating of bladder cancer cells with 5-aza-cytidine is consistent with the contention that the concordant methylation of multiple target genes is involved in the generation of DESNT cancer.

TABLE 8

| | Pearson's R | | |
|---|---|---|---|
| Pathway | squared | Pubmed ID | Description |
| TURASHVILI_BREAST_DUCTAL_CAR- | −0.683105732 | 17389037 | Genes down-regulated in ductal carcinoma vs |
| CINOMA_VS_DUCTAL_NORMAL_DN | | | normal ductal breast cells. |
| TURASHVILI_BREAST_DUCTAL_CAR- | −0.680108244 | 17389037 | Genes down-regulated in ductal carcinoma vs |
| CINOMA_ VS_LOBULAR_NORMAL_DN | | | normal lobular breast cells. |
| CHANDRAN_METASTASIS_DN | −0.676822998 | 17430594 | Genes down-regulated in metastatic tumors from |
| | | | the whole panel of patients with prostate cancer. |
| DELYS_THYROID_CANCER_DN | −0.672689295 | 17621275 | Genes down-regulated in papillary thyroid |
| | | | carcinoma (PTC) compared to normal tissue. |
| BMI1_DN.V1_DN | −0.67215877 | 17452456 | Genes down-regulated in DAOY cells |
| | | | (medulloblastoma) upon knockdown of BMI1 gene |
| | | | by RNAi. |
| TURASHVILI_BREAST_LOBULAR_CAR- | −0.666577782 | 17389037 | Genes down-regulated in lobular carcinoma vs |
| CINOMA_VS_ DUCTAL_NORMAL_DN | | | normal ductal breast cells. |
| CSR_LATE_UP.V1_DN | −0.654391638 | 14737219 | Genes down-regulated in late serum response of CRL |
| | | | 2091 cells (foreskin fibroblasts). |
| LEE_NEURAL_CREST_STEM_ CELL_DN | −0.649845872 | 18037878 | Genes down-regulated in the neural crest stem cells |
| | | | (NCS), defined as p75+/HNK1+ |
| | | | [GeneID = 4804; 27087]. |
| VECCHI_GASTRIC_CANCER_ EARLY_DN | −0.64509729 | 17297478 | Down-regulated genes distinguishing between early |
| | | | gastric cancer (EGC) and normal tissue samples. |
| GSE25088_WT_VS_STAT6_KO_MACRO- | −0.644420534 | 21093321 | Genes down-regulated in bone marrow-derived |
| PHAGE_ROSIGLIT- | | | macrophages treated with IL4 [GeneID = 3565] and |
| AZONE_AND_IL4_STIM_DN | | | rosiglitazone [PubChem = 77999]: wildtype versus |
| | | | STAT6 [GeneID = 6778] knockout. |
| WU_SILENCED_BY_METHYLA- | −0.644402585 | 17456585 | Genes silenced by DNA methylation in bladder |
| TION_IN_BLADDER_CANCER | | | cancer cell lines. |
| ACEVEDO_FGFR1_TARGETS_IN_PROS- | −0.64107159 | 18068632 | Genes down-regulated during prostate cancer |
| TATE_CANCER_MODEL_DN | | | progression in the JOCK1 model due to inducible |
| | | | activation of FGFR1 [GeneID = 2260] gene in prostate. |
| CORRE_MULTIPLE_MYELOMA_DN | −0.635300151 | 17344918 | Genes down-regulated in multiple myeloma (MM) |
| | | | bone marrow mesenchymal stem cells. |
| PEPPER_CHRONIC_LYMPHO- | −0.633518278 | 17287849 | Genes up-regulated in CD38+ [GeneID = 952] CLL |
| CYTIC_LEUKEMIA_UP | | | (chronic lymphocytic leukemia) cells. |
| POOLA_INVASIVE_BREAST_CAN- | −0.630569526 | 15864312 | Genes down-regulated in atypical ductal hyperplastic |
| CER_DN | | | tissues from patients with (ADHC) breast cancer vs |
| | | | those without the cancer (ADH). |
| GSE3982_NKCELL_VS_TH1_UP | −0.630227356 | 16474395 | Genes up-regulated in comparison of NK cells versus |
| | | | Th1 cells. |
| GO_MONOCYTE_DIFFERENTIATION | −0.629962124 | NA | The process in which a relatively unspecialized |
| | | | myeloid precursor cell acquires the specialized |
| | | | features of a monocyte. |
| LIU_PROSTATE_CANCER_DN | −0.629526171 | 16618720 | Genes down-regulated in prostate cancer samples. |
| OSADA_ASCL1_TARGETS_DN | −0.625032708 | 18339843 | Genes down-regulated in A549 cells (lung cancer) |
| | | | upon expression of ASCL1 [GeneID = 429] off a viral |
| | | | vector. |
| GAUSSMANN_MLL_AF4_FUSION_TAR- | −0.623309469 | 17130830 | Up-regualted genes from the set F (FIG. 5a): specific |
| GETS_F_UP | | | signature shared by cells expressing AF4-MLL |
| | | | [GeneID = 4299; 4297] alone and those expressing |
| | | | both AF4-MLL and MLL-AF4 fusion proteins. |

### DISCUSSION

The inventors have confirmed a key prediction of the DESNT cancer model by demonstrating that the presence of a small proportion of the DESNT cancer signature confers poor outcome. Proportion of DESNT signature could be considered as continuous variable such that as DESNT cancer content increased outcome became worse. This observation led to the development of nomograms for estimating PSA failure at 3 years, 5 years, and 7 years following prostatectomy. The result provides an extension of previous studies in which nomograms incorporating Gleason score, Stage and PSA value have been used to predict outcome following surgery²¹

The match between the 8 underlying signatures detected for the MSKCC and CancerMap datasets was used as the basis for developing a novel classification framework for human prostate cancer. A new algorithm called OAS-LPD was developed to allow rapid assessment of the presence of the 8 signatures in individual cancer samples. In total 4 clinically and or genetically distinct subgroups were identified (DESNT, S3, S4 and S5, FIG. 7). The functional significance of the new disease groupings, for example in determining drug sensitivity, remains to be established but with use of OAS-LPD it will be possible to undertake such assessments in individual patients in clinical trials. There is limited overlap between the new classification and previously proposed subgroups based on genetic alterations^{20,22-25}. However, the results may help explain conflicting results previously presented for the association of ETS status and clinical outcome²⁶. The inventors identify two subgroups, DESNT and S3, that harboured overrepresentation of ETS gene alterations. DESNT cancers have a poor prognosis, while within the S3 category cancers with ETS gene alterations have an improved outcome.

Multiplatform data (expression, mutation, and methylation data from each cancer) are available for many cancers including those present at The Cancer Genome Atlas²⁷. This has prompted the development of additional methods for sub-class discovery that can combine information from different platforms including the copula mixed model²⁸, Bayesian consensus clustering²⁹ and the iCluster model³⁰, which uses an integrative latent variable representation for each component data matrix that is present. These approaches also suffer from the problem of sample assignment to a particular cluster or group, and the failure to take into consideration the heterogeneous composition and variability of individual cancer samples. It is notable that application of OAS-LPD to mRNA expression data from TGAC¹⁷ provided a better clinical stratification of prostate cancer than application of iCluster to the entire multiplatform dataset¹⁷. These observations highlight the need to develop improved methods of analysis of multiplatform data that can take into account heterogeneity of individual prostate samples. Such approaches would have the potential to provide insights into the structure of datasets from many different cancer types using existing data.

An important issue for patients diagnosed with prostate cancer is that clinical outcome is highly heterogeneous and precise prediction of the course of progression at the time of diagnosis is not possible^{31,32}. The use of population PSA screening can reduce mortality from prostate cancer by up to 21%³³. However many, if not most, prostate cancers that are currently detected by PSA screening are clinically insignificant^{34,35}. With the increasing use of PSA testing, over-diagnosis of clinically insignificant prostate cancer is set to increase still further^{36,37}. There is therefore an urgent need for the identification of cancer categories that are associated with clinically aggressive or indolent prostate cancer to allow the targeting of radical therapies to the men that need them. For breast cancer unsupervised hierarchical clustering of transcriptome data resulted in a classification system that is routinely used to guide the management and treatment of this disease. Here the inventors provide a framework for the analysis of prostate cancer that also has its origins in unsupervised analyses of transcriptome data. Future studies will establish the utility of this classification framework in managing prostate cancer patients.

Methods

Transcriptome Datasets Eight prostate cancer microarray datasets were used that are referred to as: Memorial Sloan Kettering Cancer Centre (MSKCC), CancerMap, CamCap, Stephenson, TCGA, Klein, Erho and Karnes. The majority of samples in each dataset were obtained from tissue samples from prostatectomy patients. The CamCap dataset was produced by combining two Illumina HumanHT-12 V4.0 expression beadchip (bead microarray) datasets (GEO: GSE70768 and GSE70769) obtained from two prostatectomy series (Cambridge and Stockholm)⁷. The original CamCap⁷ and CancerMap¹⁷ datasets have 40 patients in common and thus are not independent. 20 cancer of the common cancer chosen at random were excluded from each dataset to make the two datasets independent. For the TCGA dataset, the counts per gene previously calculate were used²⁰. For the CamCap and CancerMap datasets the ERG gene alterations had been scored by fluorescence in situ hybridization^{7,17}.

TABLE 9
Transcriptome datasets.

| Dataset | Primary | Normal | Type | Platform | Citation |
|---|---|---|---|---|---|
| MSKCC⁸ | 131 | 29 | FF | Affymetrix Exon 1.0 ST v2 | Taylor et al. 2010 |
| CancerMap¹⁷ | 137 | 17 | FF | Affymetrix Exon 1.0 ST v2 | Luca et al. 2017 |
| Stephenson¹⁸ | 78 | 11 | FF | Affymetrix U133A | Stephenson et al. 2005 |
| Klein³⁸ | 182 | 0 | FFPE | Affymetrix Exon 1.0 ST v2 | Klein et al. 2015 |
| CamCap⁷ | 147 | 73 | FF | Illumina HT12 v4.0 BeadChip | Ross-Adams et al. 2015 |
| TCGA²⁰ | 333 | 43 | FF | Illumina HiSeq 2000 RNA-Seq | TCGA network 2015 |
| | | | | v2 | |
| Erho³⁹ | 545 | 0 | FFPE | Affymetrix Exon 1.0 ST v2 | Erho et al. 2013 |
| Karnes⁴⁰ | 232 | 0 | FFPE | Affymetrix Exon 1.0 ST v2 | Karnes et al. 2013 |

Each Affymetrix Exon microarray dataset was normalised using the RMA algorithm⁴¹ implemented in the Affymetrix Expression Console software. For CamCap and Stephenson previous normalised values were used¹⁷. The TCGA count data was transformed to remove the dependence of the variance on the mean using the variance stabilising transformation implemented in the DESeq2 package. Only probes corresponding to genes measured by all platforms are used (Affymetrix Exon 1.0 ST, Affymetrix U133A, RNAseq and Illumina HT12 v4.0 BeadChip). The ComBat algorithm⁴³ from the sva package, was used to mitigate series-specific effects. Additionally, quantile transformation been used to bring the intensities of all samples to the same distribution.

Latent Process Decomposition (LPD)

LPD^{13,14}, an unsupervised Bayesian approach, was used to classify samples into subgroups called processes. The inventors selected the 500 probesets with greatest variance across the MSKCC dataset for use in LPD. LPD can objectively assess the most likely number of processes. The inventors assessed the hold-out validation log-likelihood of the data computed at various number of processes and used a combination of both the uniform (equivalent to a maximum likelihood approach) and non-uniform (missed approach point approach) priors to choose the number of processes. For robustness, the inventors restarted LPD 100 times with different seeds, for each dataset. Out of the 100 runs the inventors selected a representative run that was used for subsequent analysis. The representative run was the run with the survival log-rank p-value closest to the mode.

OAS-LPD (One Added Sample LPD)

The OAS-LPD algorithm is a modified a version of the LPD algorithm in which new sample(s) are decomposed into LPD processes, without retraining the model (i.e. without re-estimating the model parameters μ_{gk}, σ²_{gk} and α in Rogers et al.¹³). Only the variational parameters Q_{kga} and γₐₖ, corresponding to the new sample(s), are iteratively updated until convergence, according to Eq. (6) and Eq. (7) from Rogers et al. 2005¹³. LPD as presented by Rogers et al.¹³ was first applied to the MSKCC dataset of 131 cancer and 29 normal samples, as described in Section Methods—LPD. The model parameters μ_{gk}, δ²_{gk} and α, corresponding to the representative LPD run, were then used to classify additional expression profiles from all datasets, one sample at a time.

Statistical Tests

All statistical tests were performed in R version 3.3.1⁸.

Correlations

Correlations between the expression profiles between two datasets for a particular gene set and sample subgroup were calculated as follows: (i) for each gene the inventors select one corresponding probeset at random; (ii) for each probeset the inventors transformed its distribution across all samples to a standard normal distribution; (iii) the average expression for each probeset across the samples in the subgroup was determined, to obtain an expression profile for the subgroup; (iv) the Pearson's correlation between the expression profiles of the subgroups in the two datasets was determined.

Differentially Expressed Features

Differentially expressed probesets were identified for each process using a moderated t-test implemented in the limma R package⁴⁴. Genes are considered significantly differentially expressed if the adjusted p-value was below 0.05 (p values adjusted using the false discovery rate). The intersect of differentially expressed genes was determined based on genes that were identified as differentially expressed in at least 50 out of 100 runs. Datasets where there were few samples assigned to a process (<10) were removed from the intersection for that process.

Differential Methylation

Differential methylation analysis was performed using the methylMix R package⁴⁵, a tool that identifies hypo and hypermethylated genes that are predictive of transcription. Only genes that were measured in all expression profiling technologies were analysed for altered methylation. A gene was considered as differentially methylated in a dataset if it was identified as functionally differentially methylated in at least 50 of 100 runs. For each process, the characteristic differentially methylated genes are only those differentially methylated genes that are also found to be differentially expressed in that process.

Survival Analyses and Nomogram

Survival analyses were performed using Cox proportional hazards models, the log-rank test, and Kaplan-Meier estimator, with biochemical recurrence after prostatectomy as the end point. For nomogram construction, the Cox proportional hazards model was fitted on the meta-dataset obtained by combining MSKCC, CancerMap and Stephenson datasets, and validated on CamCap, using the rms R package. The Gleason grade was divided into <7, 3+4, 4+3, >7, the pathological stage in T1-T2 vs. T3-T4, while DESNT percentage and PSA have been modelled as continuous covariates. The missing values for the predictors were imputed using the flexible additive models with predictive mean matching, implemented in the Hmisc R package. The linearity of the continuous covariates was assessed using the Martingale residuals⁴⁶. The lack of collinearity between covariates was determined by calculating the variance inflation factors (VIF) (VIF values between 1.04 and 3.01)⁴⁷. All covariates met the Cox proportional hazards assumption, as determined by the Schoenfeld residuals. The internal validation and calibration of the Cox model were performed by bootstrapping the training dataset 1,000 times. The calibration of the model was estimated by comparing the predicted and observed survival probabilities at 5 years. For comparing the discrimination accuracy of two non-nested Cox models the U-statistic calculated by the Hmisc rcorrp.cens function was used.

Detecting Over-Representation of Genomic Features

Mutated cancer genes identified by the Cancer Genome Atlas Research Network (2015)²⁰, were examined at the sample level. The under-/over-representation of these features in samples associated with a particular LPD process was determined using the χ² independence test.

Pathway Over-Representation Analysis

The GO biological process annotations were tested for over-representation (or under-representation) in the lists of differentially expressed genes in each OAS-LPD process, using the clusterProfiler package, version 3.4.4⁴⁸. The resulting P-values were adjusted for multiple testing using the false discovery rate (Supp Data 2).

Pathway and Signature Correlation Analysis

For a given pathway and a given sample the pathway activation score was calculated as indicated in Levine, et al.⁴⁹namely:

$${Z}_{tS} = \frac{{\overline{X}}_{tS} - {\overline{X}}_{t}}{{σ}_{t}} \sqrt{\vert S \vert}$$

where t is a tissue, S is the set of genes in the pathway, X̅_{tS} is the mean expression level of the genes in pathway S and sample t, X̅ₜ is the mean expression level of all genes in sample t, σₜ is the standard deviation of all genes in sample t, and |S| is the number of genes in the set S.

The Z-scores of all 17,697 MSigDB v6.0 gene sets were correlated with DESNT γ values, and the top 20 sets with the highest absolute Pearson's correlation were selected.

### REFERENCES

1. Edgar, R., Domrachev, M. & Lash, A. E. Gene Expression Omnibus: NCBI gene expression and hybridization array data repository. Nucleic Acids Res. 30, 207-210 (2002).
2. International Cancer Genome Consortium et al. International network of cancer genome projects. Nature 464, 993-998 (2010).
3. Ghosh, D. & Chinnaiyan, A. M. Mixture modelling of gene expression data from microarray experiments. Bioinformatics 18, 275-286 (2002).
4. Everitt, B. S., Landau, S., Leese, M. & Stahl, D. Cluster Analysis. —John Wiley & Sons. (Ltd., 2011).
5. Kohonen, T. Self-organizing maps, volume 30 of Springer Series in Information Sciences. (1995).
6. Sorlie, T. et al. Repeated observation of breast tumor subtypes in independent gene expression data sets. Proc. Natl. Acad. Sci. U.S.A. 100, 8418-8423 (2003).
7. Ross-Adams, H. et al. Integration of copy number and transcriptomics provides risk stratification in prostate cancer: A discovery and validation cohort study. EBioMedicine 2, 1133-1144 (2015).
8. Taylor, B. S. et al. Integrative genomic profiling of human prostate cancer. Cancer Cell 18, 11-22 (2010).
9. Cooper, C. S. et al. Analysis of the genetic phylogeny of multifocal prostate cancer identifies multiple independent clonal expansions in neoplastic and morphologically normal prostate tissue. Nat. Genet. 47, 367-372 (2015).
10. Boutros, P. C. et al. Spatial genomic heterogeneity within localized, multifocal prostate cancer. Nat. Genet. 47, 736-745 (2015).
11. Clark, J. et al. Complex patterns of ETS gene alteration arise during cancer development in the human prostate. Oncogene 27, 1993-2003 (2008).
12. Tsourlakis, M.-C. et al. Heterogeneity of ERG expression in prostate cancer: a large section mapping study of entire prostatectomy specimens from 125 patients. BMC Cancer 16, 641 (2016).
13. Rogers, S., Girolami, M., Campbell, C. & Breitling, R. The latent process decomposition of cDNA microarray data sets. IEEE/ACM Trans Comput Biol Bioinform 2, 143-156 (2005).
14. Carrivick, L. et al. Identification of prognostic signatures in breast cancer microarray data using Bayesian techniques. J R Soc Interface 3, 367-381 (2006).
15. Olmos, D. et al. Prognostic value of blood mRNA expression signatures in castration-resistant prostate cancer: a prospective, two-stage study. Lancet Oncol. 13, 1114-1124 (2012).
16. Blei, D. M., Ng, A. Y. & Jordan, M. I. Latent Dirichlet Allocation. Journal of Machine Learning Research 3, 993-1022 (2003).
17. Luca, B.-A. et al. DESNT: A Poor Prognosis Category of Human Prostate Cancer. European Urology Focus 0, (2017).
18. Stephenson, A. J. et al. Integration of gene expression profiling and clinical variables to predict prostate carcinoma recurrence after radical prostatectomy. Cancer 104, 290-298 (2005).
19. Hoeffding, W. A Class of Statistics with Asymptotically Normal Distribution. The Annals of Mathematical Statistics 19, 293-325 (1948).
20. Cancer Genome Atlas Research Network. The Molecular Taxonomy of Primary Prostate Cancer. Cell 163, 1011-1025 (2015).
21. Shariat, S. F., Kattan, M. W., Vickers, A. J., Karakiewicz, P. I. & Scardino, P. T. Critical review of prostate cancer predictive tools. Future Oncol 5, 1555-1584 (2009).
22. Attard, G. et al. Duplication of the fusion of TMPRSS2 to ERG sequences identifies fatal human prostate cancer. Oncogene 27, 253-263 (2008).
23. Reid, A. H. M. et al. Molecular characterisation of ERG, ETV1 and PTEN gene loci identifies patients at low and high risk of death from prostate cancer. British Journal of Cancer 102, 678-684 (2010).
24. Mosquera, J. M. et al. Concurrent AURKA and MYCN Gene Amplifications Are Harbingers of Lethal TreatmentRelated Neuroendocrine Prostate Cancer. Neoplasia 15, 1-IN4 (2013).
25. Rodrigues, L. U. et al. Coordinate loss of MAP3K7 and CHD1 promotes aggressive prostate cancer. Cancer Res. 75, 1021-1034 (2015).
26. Clark, J. P. & Cooper, C. S. ETS gene fusions in prostate cancer. Nature Reviews Urology 6, 429-439 (2009).
27. Cancer Genome Atlas Research Network et al. The Cancer Genome Atlas Pan-Cancer analysis project. Nat. Genet. 45, 1113-1120 (2013).
28. Rey, M. & Roth, V. Copula Mixture Model for Dependency-seeking Clustering. (2012).
29. Lock, E. F. & Dunson, D. B. Bayesian consensus clustering. Bioinformatics 29, 2610-2616 (2013).
30. Shen, R., Olshen, A. B. & Ladanyi, M. Integrative clustering of multiple genomic data types using a joint latent variable model with application to breast and lung cancer subtype analysis. Bioinformatics (2009).
31. D'Amico, A. V. et al. Cancer-Specific Mortality After Surgery or Radiation for Patients With Clinically Localized Prostate Cancer Managed During the Prostate-Specific Antigen Era. Journal of Clinical Oncology 21, 2163-2172 (2016).
32. Buyyounouski, M. K., Pickles, T., Kestin, L. L., Allison, R. & Williams, S. G. Validating the Interval to Biochemical Failure for the Identification of Potentially Lethal Prostate Cancer. Journal of Clinical Oncology 30, 1857-1863 (2016).
33. Schröder, F. H. et al. Screening and prostate cancer mortality: results of the European Randomised Study of Screening for Prostate Cancer (ERSPC) at 13 years of follow-up. The Lancet 384, 2027-2035 (2014).
34. Draisma, G., Etzioni, R. & Tsodikov, A. Lead time and overdiagnosis in prostate-specific antigen screening: importance of methods and context. Journal of the . . . (2009).
35. Etzioni, R., Gulati, R. & Mallinger, L. Influence of study features and methods on overdiagnosis estimates in breast and prostate cancer screening. Annals of internal . . . (2013).
36. Barry, M. J. Screening for prostate cancer—the controversy that refuses to die. N. Engl. J. Med. 360, 1351-1354 (2009).
37. Parker, C. & Emberton, M. Screening for prostate cancer appears to work, but at what cost? BJU Int. 104, 290-292 (2009).
38. Klein, E. A. et al. A Genomic Classifier Improves Prediction of Metastatic Disease Within 5 Years After Surgery in Node-negative High-risk Prostate Cancer Patients Managed by Radical Prostatectomy Without Adjuvant Therapy. Eur. Urol. 67, 778-786 (2015).
39. Erho, N. et al. Discovery and Validation of a Prostate Cancer Genomic Classifier that Predicts Early Metastasis Following Radical Prostatectomy. PLOS ONE 8, e66855 (2013).
40. Karnes, R. J. et al. Validation of a Genomic Classifier that Predicts Metastasis Following Radical Prostatectomy in an At Risk Patient Population. The Journal of Urology 190, 2047-2053 (2013).
41. Irizarry, R. A. et al. Exploration, normalization, and summaries of high density oligonucleotide array probe level data. Biostatistics 4, 249-264 (2003).
42. Love, M. I., Huber, W. & Anders, S. Moderated estimation of fold change and dispersion for RNA-seq data with DESeq2. Genome Biol. 15, 550 (2014).
43. Johnson, W. E., Li, C. & Rabinovic, A. Adjusting batch effects in microarray expression data using empirical Bayes methods. Biostatistics 8, 118-127 (2007).
44. Ritchie, M. E., Phipson, B., Wu, D. & Hu, Y. limma powers differential expression analyses for RNA-sequencing and microarray studies. Nucleic acids . . . (2015).
45. Gevaert, O. MethylMix: an R package for identifying DNA methylation-driven genes. Bioinformatics (2015).
46. Therneau, T. M., Grambsch, P. M. & Fleming, T. R. Martingale-based residuals for survival models. Biometrika (1990).
47. Hair, J. F., Black, W. C., Babin, B. J., Anderson, R. E. & Tatham, R. L. Multivariate data analysis. (1998).
48. Yu, G., Wang, L.-G., Han, Y. & He, Q.-Y. clusterProfiler: an R Package for Comparing Biological Themes Among Gene Clusters. OMICS: A Journal of Integrative Biology 16, 284-287 (2012).
49. Levine, D. M. et al. Pathway and gene-set activation measurement from mRNA expression data: the tissue distribution of human pathways. Genome Biol. 7, R93 (2006).

### EMBODIMENTS

The present invention provides at least the follow embodiments:
1. A method of classifying prostate cancer or predicting prostate cancer progression in a patient, comprising:
   a) providing a set of reference parameters, wherein the reference parameters are obtained from a Latent Process Decomposition (LPD) analysis performed on a reference dataset, the reference dataset comprising A expression profiles, each expression profile comprising the expression status of G genes, wherein the reference dataset is decomposed using the LPD analysis into K different cancer expression signatures;
   b) obtaining or providing the expression status of G genes in a sample obtained from the patient to provide a patient expression profile, wherein the G genes in the patient expression profile are the same genes of the reference dataset used to provide the set of reference parameters; and
   c) classifying the prostate cancer or predicting cancer progression by determining the contribution of each different cancer expression signature to the patient expression profile using the set of reference parameters provided in step (a).
2. The method of embodiment 1, wherein the step of classifying the cancer comprises determining the cancer classification that contributes the most to the patient expression profile and assigning the patient cancer to that cancer classification.
3. The method of any preceding embodiment, wherein providing a set of reference parameters comprises:
   a) providing the reference dataset comprising A expression profiles and G genes for each expression profile;
   b) performing LPD analysis on the reference dataset to classify each expression profiles into K cancer classifications.
4. The method of embodiment 3, wherein step (b) is repeated at least 2, at least 10, at least 25, at least 50 or at least 100 times.
5. The method of any preceding embodiment, wherein the reference parameters are derived from a representative LPD analysis carried out on a reference dataset.
6. The method of step 5, wherein the representative LPD analysis is the LPD run with the survival log-rank p-value closest to the modal value.
7. The method of any preceding embodiment, wherein K is determined empirically during the LPD composition.
8. The method of any preceding embodiment, wherein K is 8.
9. The method of any preceding embodiment, wherein A is at least 100 and G is at least 100.
10. The method of any preceding embodiment, wherein the G is at least 100 and the genes are selected from Table 1.
11. The method of any preceding embodiment, wherein G is at least 500 and the genes are selected from the genes of Table 1.
12. The method of any preceding embodiment, wherein the reference parameters are:
   a) α—a variable that specifies a Dirichlet distribution in K dimensions, where K is the number of cancer signatures;
   b) μ—a set of G by K variables, denoted μ_{gk}, storing the means of G×K Gaussian components; and
   c) σ—a set of G by K variables, denoted σ_{gk}, storing the variances of G×K Gaussian components, wherein each pair μ_{gk}, σ_{gk} defines the normal distribution that encodes the distribution of expression levels of a given gene in a given cancer signature K.
13. The method of embodiment 12, wherein a defines the probability of occurrence of each cancer signature in the reference dataset.
14. The method of embodiment 12 or embodiment 13, wherein a defines the probably of co-occurrence of each cancer signature in the reference dataset.
15. The method of any preceding embodiment, wherein the reference parameters define a gene expression profile for each cancer expression signature K.
16. The method of any preceding embodiment, wherein the step of classifying the cancer or predicting cancer progression comprises splitting the patient expression profile between the gene expression profile for each cancer expression signature.
17. The method of any preceding embodiment, wherein the method comprises normalising the patient expression profile to the expression profiles of the reference dataset prior to classifying the cancer.
18. The method of any preceding embodiment, wherein the patient expression profile is provided as an RNA expression profile or a cDNA expression profile.
19. The method of any preceding embodiment, wherein each cancer classification K is defined according to its gene expression profile, gene mutation profile and/or the clinical outcome of the cancer.
20. The method of any preceding embodiment, wherein the cancer is prostate cancer and K is 7,8 or 9, wherein the prostate cancer classifications include the following classifications:
   a) Upregulation of one or more of KRT13 and TGM4;
   b) Upregulation of one or more of CSGALNACT1, ERG, GHR, GUCY1A3, HDAC1, ITPR3 and PLA2G7 and optionally an increase in the number of mutation in one or more of SPOP and CHD1 and/or a decrease in the number of mutations in one or more of ERG and PTEN;
   c) Upregulation of one or more of ABHD2, ACAD8, ACLY, ALCAM, ALDH6A1, ALOX15B, ARHGEF7, AUH, BBS4, C1orf115, CAMKK2, COGS, CPEB3, CYP2J2, DHX32, EHHADH, ELOVL2, EXTL2, FAM111A, GLUD1, GNMT, HPGD, MIPEP, MON1B, NANS, NAT1, NCAPD3, PPFIBP2, PTPN13, PTPRM, RAB27A, REPS2, RFX3, SCIN, SLC1A1, SLC4A4, SMPDL3A, STXBP6, SYTL2, TBPL1, TFF3, TUBB2A, and YIPF1 and/or downregulation of one or more of DHRS3, ERG, F3, GATA3, HES1, KHDRBS3, LAMB2, LAMC2, PDE8B, PTK7, SORL1, TRIM29 and ZNF516; and optionally an increase in the number of mutation in one or more of ERG and PTEN and/or a decrease in the number of mutations in one or more of SPOP and CHD1;
   d) Upregulation of one or more of CCL2, CFB, CFTR, CXCL2, 1F116, LCN2, LTF, LXN, TFRC;
   e) Upregulation of one or more of F5 and KHDRBS3, and downregulation of one or more of ACTG2, ACTN1, ADAMTS1, ANPEP, ARMCX1, AZGP1, C7, CD44, CHRDL1, CNN1, CRISPLD2, CSRP1, CYP27A1, CYR61, DES, EGR1, ETS2, FBLN1, FERMT2, FHL2, FLNA, FXYD6, FZD7, ITGA5, ITM2C, JAM3, JUN, LMOD1, LPHN2, MT1M, MYH11, MYL9, NFIL3, PARM1, PCP4, PDK4, PLAGL1, RAB27A, SERPINF1, SNAI2, SORBS1, SPARCL1, SPOCK3, SYNM, TAGLN, TCEAL2, TGFB3, TPM2, VCL; and optionally an increase in the number of mutation in one or more of ERG and PTEN; and/or
   f) Upregulation of one or more of ARHGEF6, AXL, CD83, COL15A1, DPYSL3, EPB41 L3, FBN1, FCHSD2, FHL1, FXYD5, GNAO1, GPX3, 1F116, IRAK3, ITGA5, LAPTM5, MFAP4, MFGE8, MMP2, PARVA, PLEKHO1, PLSCR4, RFTN1, SAMD4A, SAMSN1, SERPINF1, VCAM1, WIPF1 and ZYX and/or downregulation of one or more of ABCC4, ACAT2, ATP8A1, CANT1, CDH1, DCXR, DHCR24, DHRS7, FAM174B, FAM189A2, FKBP4, FOXA1, GOLM1, GTF3C1, HPN, KIF5C, KLK3, MAP7, MBOAT2, MIOS, MLPH, MYO5C, NEDD4L, PART1, PDIA5, PIGH, PMEPA1, PRSS8, SEC23B, SLC43A1, SPDEF, SPINT2, STEAP4, TMPRSS2, TRPM8, TSPAN1, XBP1.
21. The method according to any preceding embodiment, wherein one or more of the cancer classifications are associated with a cancer prognosis
22. The method of any preceding embodiment, wherein K is 7, 8 or 9, and wherein at least one of the prostate cancer classifications is associated with a poor prognosis.
23. The method of embodiment 21, wherein at least one of the prostate cancer classifications is associated with a poor prognosis and is further associated with upregulation of one or more of F5 and KHDRBS3, and/or downregulation of one or more of ACTG2, ACTN1, ADAMTS1, ANPEP, ARMCX1, AZGP1, C7, CD44, CHRDL1, CNN1, CRISPLD2, CSRP1, CYP27A1, CYR61, DES, EGR1, ETS2, FBLN1, FERMT2, FHL2, FLNA, FXYD6, FZD7, ITGA5, ITM2C, JAM3, JUN, LMOD1, LPHN2, MT1 M, MYH11, MYL9, NFIL3, PARM1, PCP4, PDK4, PLAGL1, RAB27A, SERPINF1, SNAI2, SORBS1, SPARCL1, SPOCK3, SYNM, TAGLN, TCEAL2, TGFB3, TPM2, VCL, and optionally an increase in the number of mutation in one or more of ERG and PTEN.
24. The method of any preceding embodiment, wherein K is 7, 8 or 9, and wherein at least one of the prostate cancer classifications is associated with a good prognosis.
25. The method of any preceding embodiment, further comprising assigning a unique label to the patient expression profile prior to statistical analysis.
26. The method of any preceding embodiment, wherein the contribution of each cancer expression signature to the patient expression profile is a continuous variable.
27. The method of any preceding embodiment, wherein one or more of the cancer expression signatures are correlated with one or more properties, and the level of contribution of a given cancer expression signature to a patient's expression profile determines the degree to which the patient's cancer exhibits the corresponding property.
28. A method of classifying cancer or predicting cancer progression, comprising:
   a) providing one or more reference datasets where the cancer classification of each patient sample in the datasets is known;
   b) selecting from this dataset a plurality of genes;
   c) applying a LASSO logistic regression model analysis on the selected genes to identify a subset of the selected genes that are predictive of each cancer classification;
   d) using the expression status of this subset of selected genes to apply a supervised machine learning algorithm on the dataset to obtain a predictor for each cancer classification;
   e) providing the expression status of the subset of selected genes in a sample obtained from the patient to provide a patient expression profile;
   f) optionally normalising the patient expression profile to the reference dataset(s); and
   g) applying the predictor to the patient expression profile to classify the cancer or predict cancer progression.
29. The method of embodiment 28, wherein at least 10,000 genes are selected in step (b).
30. The method of embodiment 28 or embodiment 29, wherein the expression status of the genes selected in step (b) are known to vary between cancer classifications.
31. The method of any one of embodiments 28 to 30, wherein the plurality of genes selected in step (b) comprises at least 1000, at least 5000, or at least 10,000 genes from the human genome.
32. The method of any one of embodiments 28 to 31, wherein the supervised machine learning algorithm is a random forest analysis.
33. A method of classifying cancer or predicting cancer progression, comprising:
   a) providing one or more reference datasets where the cancer classification of each patient sample in the datasets is known;
   b) selecting from this dataset a plurality of genes, wherein the plurality of genes comprises at least 5, at least 10, at least 20, at least 30, at least 40, at least 50, at least 100, or at least 150 genes or all the genes selected from the group listed in Table 2
   c) optionally:
      i. determining the expression status of at least 1 further, different, gene in the patient sample as a control, wherein the control gene is not a gene listed in Table 2; and
      ii. determining the relative levels of expression of the plurality of genes and of the control gene(s);
   d) using the expression status of those selected genes to apply a supervised machine learning algorithm on the dataset to obtain a predictor for each cancer classification;
   e) providing the expression status of the same plurality of genes in a sample obtained from the patient to provide a patient expression profile;
   f) optionally normalising the patient expression profile to the reference dataset; and
   g) applying the predictor to the patient expression profile to classify the cancer, or to predict cancer progression.
34. The method of embodiment 33, wherein determining the relative levels of expression comprises determining a ratio of expression for each pair of genes in the patient dataset and the reference dataset.
35. The method of any one of embodiments 33 or 34, wherein the machine learning algorithm is a random forest analysis.
36. The method of any one of embodiments 33 to 35, wherein the at least 1 control gene is a gene listed in Table 3 or Table 4.
37. The method of any one of embodiments 33 to 36, wherein expression status of at least 2 control genes is determined.
38. A method of classifying cancer or predicting cancer progression, comprising:
   a) providing a reference dataset wherein the cancer classification of each patient sample in the dataset is known;
   b) selecting from this dataset of a plurality of genes;
   c) using the expression status of those selected genes to apply a supervised machine learning algorithm on the dataset to obtain a predictor for cancer classification;
   d) determining the expression status of the same plurality of genes in a sample obtained from the patient to provide a patient expression profile;
   e) optionally normalising the patient expression profile to the reference dataset; and
   f) applying the predictor to the patient expression profile to classify the cancer, or to predict cancer progression.
39. The method according to embodiment 38, wherein the supervised machine learning algorithm is a random forest analysis.
40. A method according to any one of embodiments 38 or 39, wherein at least 100, at least 200, or at least 500 genes from the human genome are selected in step b).
41. A method according to any preceding embodiment, wherein the sample is a urine sample, a semen sample, a prostatic exudate sample, or any sample containing macromolecules or cells originating in the prostate, a whole blood sample, a serum sample, saliva, or a biopsy.
42. The method of embodiment 41, wherein the sample is a prostate biopsy, prostatectomy or TURP sample.
43. A method according to any preceding embodiment, further comprising obtaining a sample from a patient.
44. A method according to any preceding embodiment, wherein the method is carried out on at least 2, at least 3, at least 3 or at least 5 samples.
45. A method according to any preceding embodiment wherein the reference dataset or datasets comprise a plurality of tumour or patient expression profiles.
46. The method of embodiment 45, wherein the datasets each comprise at least 20, at least 50, at least 100, at least 200, at least 300, at least 400 or at least 500 patient or tumour expression profiles.
47. The method of embodiment 45 or embodiment 46, wherein the patient or tumour expression profiles comprise information on the expression status of at least 10, at least 40, at least 100, at least 500, at least 1000, at least 1500, at least 2000, at least 5000 or at least 10000 genes.
48. The method of embodiment 45 or 46, wherein the patient or tumour expression profiles comprise information on the levels of expression of at least 10, at least 40, at least 100, at least 500, at least 1000, at least 1500, at least 2000, at least 5000 or at least 10000 genes.
49. A method of treating cancer, comprising administering a treatment to a patient that has undergone a diagnosis or classification according to the method of any one of embodiments 1 to 48.
50. The method of embodiment 49, comprising:
   a) providing a patient sample;
   b) predicting cancer progression, predicting treatment responsiveness or classifying cancer according to method as defined in any one of embodiments 1 to 48; and
   c) administering to the patient a treatment for cancer if cancer progression is predicted, detected or suspected according to the results of the prediction in step b), or if the patient is predicted as being responsive to the treatment.
51. A method of diagnosing cancer, comprising predicting cancer progression or classifying cancer according to a method as defined in any one of embodiments 1 to 48.
52. A computer apparatus configured to perform a method according to any one of embodiments 1 to 48.
53. A computer readable medium programmed to perform a method according to any one of embodiments 1 to 48.
54. A biomarker panel, comprising at least 75% of the genes listed in Table 2 or 75% of the genes listed in one of biomarker panels A to F.
55. A biomarker panel, comprising at least all of the genes listed in Table 2 or all of the genes listed in one of biomarker panels A to F.
56. Use of a biomarker panel according to embodiment 54 or embodiment 55 in a method of diagnosing or prognosing cancer, a method of predicting cancer progression, or a method of classifying cancer, or a method of predicting a patient's responsiveness to a cancer treatment.
57. A method of diagnosing or prognosing cancer, or a method of predicting cancer progression, or a method of classifying cancer, comprising determining the level of expression or expression status of one or more of the genes in any one of biomarker panels of embodiment 54 or embodiment 55.
58. The method of embodiment 57, wherein the method comprises determining the level of expression or expression status of all of the genes in one of the biomarker panels of embodiment 53 or embodiment 54.
59. The method of embodiment 57 or 58, further comprising comparing the level of expression or expression status of the measured biomarkers with one or more reference genes.
60. The method of embodiment 59, wherein the one or more reference genes is/are a housekeeping gene(s).
61. The method of embodiment 60, wherein the housekeeping genes is/are selected from the genes in Table 3 or Table 4.
62. The method of any one of embodiments 57 to 61, wherein the method comprises comparing the levels of expression or expression status of the same gene or genes in a sample from a healthy patient or a patient that does not have cancer.
63. A kit comprising means for detecting the level of expression or expression status of at least 5 genes from a biomarker panel as defined in embodiment 54 or 55.
64. A kit comprising means for detecting the level of expression or expression status of all of the genes from a biomarker panel as defined in embodiment 54 or 55
65. The kit of embodiment 63 or embodiment 64, further comprising means for detecting the level of expression or expression status of one or more control or reference genes
66. A kit of any one of embodiments 63 to 65, further comprising instructions for use.
67. A kit of any one of embodiments 63 to 66, further comprising a computer readable medium as defined in embodiment 53.

1. A method of classifying prostate cancer or predicting prostate cancer progression in a patient, comprising:
a) providing a set of reference parameters, wherein the reference parameters are obtained from a Latent Process Decomposition (LPD) analysis performed on a reference dataset, the reference dataset comprising A expression profiles, each expression profile comprising the expression status of G genes, wherein the reference dataset is decomposed using the LPD analysis into K different cancer expression signatures, wherein K is 8;
b) obtaining or providing the expression status of G genes in a sample obtained from the patient to provide a patient expression profile, wherein the G genes in the patient expression profile are the same genes of the reference dataset used to provide the set of reference parameters; and
c) classifying the prostate cancer or predicting cancer progression by determining the contribution of each different cancer expression signature to the patient expression profile using the set of reference parameters provided in step (a);
wherein the method does not comprise a step of conducting LPD analysis on the reference dataset.

2. The method of claim 1, wherein the step of classifying the cancer comprises determining the cancer classification that contributes the most to the patient expression profile and assigning the patient cancer to that cancer classification.

3-4. (canceled)

5. The method of claim 1, wherein the reference parameters are derived from a representative LPD analysis carried out on a reference dataset, optionally wherein the representative LPD analysis is the LPD run with the survival log-rank p-value closest to the modal value; A is at least 100 and G is at least 100; and/or G is at least 500 and optionally the genes are selected from the genes of Table 1.

6-9. (canceled)

10. The method of claim 1, wherein the reference parameters are:
a) α—a variable that specifies a Dirichlet distribution in K dimensions, where K is the number of cancer expression signatures;
b) μ—a set of G by K variables, denoted μ_{gk}, storing the means of G×K Gaussian components; and
c) σ—a set of G by K variables, denoted σ_{gk}, storing the variances of G×K Gaussian components, wherein each pair μ_{gk},σ_{gk} defines the normal distribution that encodes the distribution of expression levels of a given gene in a given cancer signature K.

11. The method of claim 10, wherein a defines the probability of occurrence of each cancer signature in the reference dataset.

12. The method of claim 11, wherein a defines the probably of co-occurrence of each cancer signature in the reference dataset.

13. The method of any claim 1, wherein the reference parameters define a gene expression profile for each cancer expression signature K.

14. The method of claim 1, wherein the step of classifying the cancer or predicting cancer progression comprises splitting the patient expression profile between the gene expression profile for each cancer expression signature.

15. The method of claim 1, wherein the method comprises normalising the patient expression profile to the expression profiles of the reference dataset prior to classifying the cancer.

16. The method of claim 1, wherein each cancer classification K is defined according to its gene expression profile, gene mutation profile and/or the clinical outcome of the cancer.

17. The method of claim 1, wherein the cancer is prostate cancer wherein the prostate cancer classifications include the following classifications:
a) upregulation of one or more of KRT13 and TGM4;
b) upregulation of one or more of CSGALNACT1, ERG, GHR, GUCY1A3, HDAC1, ITPR3 and PLA2G7 and optionally an increase in the number of mutation in one or more of SPOP and CHD1 and/or a decrease in the number of mutations in one or more of ERG and PTEN;
c) upregulation of one or more of ABHD2, ACAD8, ACLY, ALCAM, ALDH6A1, ALOX15B, ARHGEF7, AUH, BBS4, Clorf115, CAMKK2, COGS, CPEB3, CYP2J2, DHX32, EHHADH, ELOVL2, EXTL2, FAM111A, GLUD1, GNMT, HPGD, MIPEP, MON1B, NANS, NAT1, NCAPD3, PPFIBP2, PTPN13, PTPRM, RAB27A, REPS2, RFX3, SCIN, SLC1A1, SLC4A4, SMPDL3A, STXBP6, SYTL2TBPL1TFF3, TUBB2A, and YIPF1 and/or downregulation of one or more of DHRS3, ERG, F3, GATA3, HES1, KHDRBS3, LAMB2, LAMC2, PDE8B, PTK7, SORL1, TRIM29 and ZNF516; and optionally an increase in the number of mutation in one or more of ERG and PTEN and/or a decrease in the number of mutations in one or more of SPOP and CHD1;
d) upregulation of one or more of CCL2, CFB, CFTR, CXCL2, IFI16, LCN2, LTF, LXN, TFRC;
e) upregulation of one or more of F5 and KHDRBS3, and/or downregulation of one or more of ACTG2, ACTN1, ADAMTS1, ANPEP, ARMCX1, AZGP1, C7, CD44, CHRDL1, CNN1, CRISPLD2, CSRP1, CYP27A1, CYR61, DES, EGR1, ETS2, FBLN1, FERMT2, FHL2, FLNA, FXYD6, FZD7, ITGA5, ITM2C, JAM3, JUN, LMOD1, LPHN2, MT1M, MYH11, MYL9, NFIL3, PARM1, PCP4, PDK4, PLAGL1, RAB27A, SERPINF1, SNAI2, SORBS1, SPARCL1, SPOCK3, SYNM, TAGLN, TCEAL2, TGFB3, TPM2, VCL; and optionally an increase in the number of mutation in one or more of ERG and PTEN; and/or
f) upregulation of one or more of ARHGEF6, AXL, CD83, COL15A1, DPYSL3, EPB41L3, FBN1, FCHSD2, FHL1, FXYD5, GNAO1, GPX3, IRAK3, ITGA5, LAPTM5, MFAP4, MFGE8, MMP2, PARVA, PLEKHO1, PLSCR4, RFTN1, SAMD4A, SAMSN1, SERPINF1, VCAM1, WIPF1 and ZYX and/or downregulation of one or more of ABCC4, ACAT2, ATP8A1, CANT1, CDH1, DCXR, DHCR24, DHRS7, FAM174B, FAM189A2, FKBP4, FOXA1, GOLM1, GTF3C1, HPN, KIF5C, KLK3, MAP7, MBOAT2, MIOS, MLPH, MYO5C, NEDD4L, PART1, PDIA5, PIGH, PMEPA1, PRSS8, SEC23B, SLC43A1, SPDEF, SPINT2, STEAP4, TMPRSS2, TRPM8, TSPAN1, XBP1.

18. (canceled)

19. The method of claim 1, wherein at least one of the prostate cancer classifications is associated with a poor prognosis.

20. The method of claim 19, wherein the at least one prostate cancer classification associated with a poor prognosis is further associated with upregulation of one or more of F5 and KHDRBS3, and/or downregulation of one or more of ACTG2, ACTN1, ADAMTS1, ANPEP, ARMCX1, AZGP1, C7, CD44, CHRDL1, CNN1, CRISPLD2, CSRP1, CYP27A1, CYR61, DES, EGR1, ETS2, FBLN1, FERMT2, FHL2, FLNA, FXYD6, FZD7, ITGA5, ITM2C, JAM3, JUN, LMOD1, LPHN2, MT1M, MYH11, MYL9, NFIL3, PARM1, PCP4, PDK4, PLAGL1, RAB27A, SERPINF1, SNAI2, SORBS1, SPARCL1, SPOCK3, SYNM, TAGLN, TCEAL2, TGFB3, TPM2, VCL, and optionally an increase in the number of mutation in one or more of ERG and PTEN.

21. The method of claim 1, wherein at least one of the prostate cancer classifications is associated with a good prognosis.

22. The method of claim 1, wherein the contribution of each cancer expression signature to the patient expression profile is a continuous variable.

23. The method of claim 1, wherein one or more of the cancer expression signatures are correlated with one or more properties, and the level of contribution of a given cancer expression signature to a patient's expression profile determines the degree to which the patient's cancer exhibits the corresponding property

24-26. (canceled)

27. The method of claim 1 wherein the reference dataset comprises at least 500 patient or tumour expression profiles.

28. The method of claim 27, wherein the patient or tumour expression profiles comprise information on the expression status of at least 10000 genes.

29. (canceled)

30. A computer apparatus configured to perform a method according to claim 1 or, a computer readable medium programmed to perform a method according to claim 1.

31-39. (canceled)

40. A kit comprising means for detecting the level of expression or expression status of at least 5 genes from a biomarker panel comprising at least 75% of the genes listed in Table 2 or 75% of the genes listed in one of biomarker panels A to F, and optionally further comprising means for detecting the level of expression or expression status of one or more control or reference genes and further optionally comprising a computer readable medium as defined in claim 30.

41. (canceled)
